(19) United States
(12) Patent Application Publication
(10) Pub. No.: US 2026/0337192 A1
(43) Pub. Date: Oct. 29, 2026

(54) COMPOUNDS COMPRISING N-METHYL-2-PYRIDONE, AND PHARMACEUTICALLY ACCEPTABLE SALTS

(71) Applicant: Tay Therapeutics Limited, Dundee (GB)

(72) Inventors: Christopher Andrew WOODLAND, Dundee (GB); Mark BELL, Dundee (GB)

(21) Appl. No.: 19/443,210

(22) Filed: Jan. 8, 2026

Related U.S. Application Data
(63) Continuation of application No. 19/225,743, filed on Jun. 2, 2025, now abandoned.
Application No. 19/225,743 is a continuation of application No. 17/594,577, filed on Apr. 22, 2020, now Pat. No. 12,384,777.

(30) Foreign Application Priority Data
Apr. 24, 2019 (GB) 1905721.5

(51) Int. Cl. C07D 471/04; C07D 401/04; C07D 413/14

(52) U.S. Cl. CPC C07D 471/04; C07D 401/04; C07D 413/14

(57) ABSTRACT

The present invention concerns compounds comprising N-methyl-2-pyridone, and pharmaceutically-acceptable salts and compositions of such compounds. Such compounds are useful in anti-inflammatory and anti-cancer therapies. Therefore, the present invention also concerns such compounds for use as medicaments, particularly for the treatment of inflammatory diseases and oncology.

### RELATED APPLICATIONS

This application is a continuation of U.S. application Ser. No. 19/225,743, filed Jun. 2, 2025, which is a continuation of U.S. application Ser. No. 17/594,577, filed Oct. 22, 2021, which is a U.S. National Stage Application under 35 U.S.C. § 371 of International Application No. PCT/EP2020/061173, filed Apr. 22, 2020, which claims priority to, and the benefit of, GB Application No. 1905721.5, filed Apr. 24, 2019, the contents of each of which are incorporated herein by reference in their entireties for all purposes.

### FIELD OF THE INVENTION

The present invention concerns compounds comprising N-methyl-2-pyridone, and pharmaceutically-acceptable salts and compositions of such compounds. The compounds of the invention are useful as anti-inflammatory and anti-cancer therapies. Therefore, the present invention also concerns compounds comprising N-methyl-2-pyridone for use as medicaments, particularly for the treatment of inflammatory diseases and oncology.

### BACKGROUND OF THE INVENTION

Bromodomain and Extra-Terminal (BET) proteins are a family of four bromodomain-containing (BRD) proteins (BRD2, BRD3, BRD4 and BRDT). All four members contain two BRDs (located next to each other toward the N-terminal of the proteins) and an extra-terminal domain (Shi, J. et al. Cancer Cell 25 (2): 210-225 (2014)). The two BRDs in each BET protein are designated binding domain I (BDI) and binding domain II (BDII). The BRD is a functional protein domain that contains a defined and predominantly hydrophobic pocket that binds to acetylated lysine residues, typically those found on transcription factors (Shi, J. et al. Cancer Cell 25 (2): 210-225 (2014)) or on the N-terminal tails of histone proteins. BRDs function as epigenetic regulators, i.e. they functionally alter gene activity and expression without altering the DNA sequence. For example, BRD4 recruits the transcription factor P-TEFb to promoters leading to altered expression of genes involved in the cell cycle (Yang et al., Mol. Cell Biol. 28:967-976 (2008)). BRD2 and BRD3 also regulate growth promoting genes (LeRoy et al., Mol Cell 30:51-60 (2008)). Therefore, BRDs are responsible for transducing the signals carried by acetylated lysine residues into various phenotypes. BETs are considered in the art to be ubiquitously expressed in humans except for BRDT, which is normally expressed in the testes but is also expressed by some cancers (Ekaterina B. F. et al. Cell J. 19 (Suppl 1): 1-8 (2017)).

BET proteins have roles in the regulation of a number of pathways such as MYC, BCL2, FOSL1, P-TEFb, NFkB, Glucocorticoid signalling and others (Shi J. et al. Mol Cell. June 5; 54 (5): 728-36 (2014)), (Hajmirza A. Biomedicines. February 6; 6 (1). pii: E16 (2018)), (Shan N. Elife. September 11; 6. pii: e27861. (2017)), (Huang B. Mol Cell Biol. Mar; 29 (5): 1375-87 (2009)). As such, BET inhibitors are considered to have potential uses in a range of inflammatory diseases, cancers, infections, metabolic diseases, CNS disorders, fibrotic diseases and cardiac diseases (Deanna A. M. et al. J Exp Med. October 21; 210 (11): 2181-2190 (2013)), (Rab K. P. et al. Trends Pharmacol. Sci. Mar; 33 (3): 146-53 (2012)), (Anna C. B. et al. J Immunol. April 1; 190 (7): 3670-3678 (2013)), (Zuber J. et al. Nature. August 3; 478 (7370): 524-8. (2011)), (Montserrat P. S. et al. Epigenetics.; 12 (5): 323-339 (2017)), (Qiming D. et al. Sci Transl Med. May 17; 9 (390): eaah5084. (2017)), (Kristin M. K et al. J Biol Chem. August 11; 292 (32): 13284-13295 (2017)), (Ning D. et al. PNAS December 22, 112 (51) 15713-15718 (2015)).

Compounds that can inhibit or affect the function of BET proteins have the potential to modulate gene expression and treat diseases that are at least in part caused by abnormal regulation of BET protein activity. Several small molecules have been reported to be effective in BET inhibition, including diazepine-, 3,5-dimethylisoxazole-, thiazol-2-one-, diazobenzene-, and 4-acylpyrrole-based compounds (see M. Brand et al, ACS Chem. Biol. 2015, 10, 22-39, WO2011054553, WO2011054845). Compounds that can selectively inhibit the function of BDII over BDI have the potential to modulate gene expression and treat diseases that are at least in part caused by abnormal regulation of BET protein activity while offering the potential of an improved therapeutic index. Improved therapeutic index and pre-clinical safety of BDII selective BET inhibitors verses pan-BET inhibitors has been demonstrated (E. Faivre et al. Nature 578, 306-310 (2020)).

Compounds comprising 6-methyl-1,6-dihydro-7H-pyrrolo[2,3-c]pyridin-7-one moieties, substituted at the 4- and/or 2-positions are described in patent applications WO 2017177955, WO 2016077378, WO 2015081280, WO 2014206150, WO 2014206345, WO 2013097601, WO 2013097052 and WO 2018130174 as useful for the inhibition of BET proteins.

The present invention provides alternative BET protein inhibitors useful in the treatment or prophylaxis of the conditions described herein.

### SUMMARY OF THE INVENTION

It has been found that the compounds and compositions of this teaching are surprisingly active in inhibiting all four BET BRDs, with effective potency at nanomolar concentrations. The compounds and compositions are highly soluble in a range of solvents and formulations suitable for topical and/or oral application. Advantageously, many of the compounds and compositions of the invention are stable in human skin and under hydrolytic conditions at a range of pH values. Furthermore, formulations of the compounds and compositions may deliver practicable concentrations of the compound into the epidermis of the skin and the compounds are not toxic to skin cells. Some of the compounds and compositions exhibit surprisingly effective clearance by the liver, offering potential use as medicaments with a lower risk of side-effects. Other compounds and compositions are surprisingly stable, offering potential use as medicaments for oral administration. Some of the compounds are surprisingly selective for BDII over BDI offering the potential of an improved therapeutic index and a lower risk of side-effects.

The skilled person is aware that any reference to an aspect of the current disclosure includes every embodiment of that aspect. For example, any reference to the first aspect includes the first aspect and all embodiments of the first aspect.

Viewed from a first aspect, there is provided a compound of formula (I):

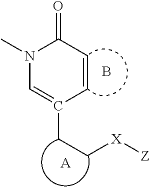

wherein ring structure A is a 5- or 6-membered aromatic or heteroaromatic ring, optionally substituted at one or more carbon and/or heteroatoms with a first substituent;
wherein each first substituent is independently selected from the group consisting of hydroxy, oxo, C₁-C₆alkyl, C₃-C₆cycloalkyl, C₁-C₆alkylol, halo, SO₂C₁-C₄alkyl, NHSO₂C₁-C₄alkyl, SO₂C₃-C₆cycloalkyl, NHSO₂C₃-C₆cycloalkyl, SO₂C₁-C₄alkylol, NHSO₂C₁-C₄alkylol, C₁-C₅alkyloxy, C₁-C₅alkylamino, SO₂NH₂, CONH₂, CONHC₁-C₄alkyl, NHCOC₁-C₄alkyl, NHSO₂N(C₁-C₄alkyl)₂, C₁-C₆fluoroalkyl, SO₂C₁-C₄fluoroalkyl, NHSO₂C₁-C₄fluoroalkyl, C₁-C₅fluoroalkyloxy, and C₁-C₅fluoroalkylamino;
X is O, CR², NR′ or S, wherein R is individually selected from the group consisting of H, C₁-C₄alkyl and halo, and R′ is selected from the group consisting of C₁-C₄alkyl and H;
Z is a 5- or 6-membered aromatic or heteroaromatic ring, C₁-C₆alkyl, C₃-C₆cycloalkyl, CR^{A}R^{B}R^{C}, C₂-C₅oxacycloalkyl, C₂-C₅azacycloalkyl or morpholinyl, optionally substituted at one or more carbon and/or heteroatoms with a second substituent;
wherein R^{A} is a C₃-C₅cycloalkyl, R^{B} is a C₃-C₅cycloalkyl, methyl or ethyl, and R^{C} is OH; and
each second substituent is independently selected from the group consisting of hydroxy, C₁-C₆alkyl, C₃-C₆cycloalkyl, halo, C₁-C₅alkyloxy, C₁-C₅alkylamino, oxo, cyano, C₁-C₆fluoroalkyl, C₁-C₅fluoroalkyloxy, and C₁-C₅fluoroalkylamino;
ring structure B is optionally present; wherein when ring structure B is present, it is an optionally substituted pyrrole bonded such that C is in the 4 position relative to NH; wherein the pyrrole is optionally substituted at position 2 with a third substituent;
wherein the third substituent is selected from the group consisting of CONHC₁-C₄alkyl, CONH₂, CONHC₁-C₆fluoroalkyl, CONHC₃-C₆cycloalkyl optionally substituted at one or more carbon atoms with a methyl or ethyl; CONHC₃-C₅cyclofluoroalkyl optionally substituted at one or more carbon atoms with a methyl or ethyl, NHCOC₁-C₄alkyl and NHCOC₁-C₄fluoroalkyl;
with the proviso that when A is 6-membered, it is substituted at least once with a hydroxy or oxo group.

Viewed from a second aspect, there is provided a pharmaceutical composition comprising any one or a combination of the compounds defined in the first aspect, in combination with one or more pharmaceutically acceptable excipients.

Viewed from a third aspect, there is provided a compound as defined in the first aspect or a pharmaceutical composition as defined in the second aspect, for use as a medicament.

Viewed from a fourth aspect, there is provided a compound as defined in the first aspect, or a pharmaceutical composition as defined in the second aspect, for use in a method of treatment or prophylaxis of inflammatory skin disorders, respiratory diseases, gastrointestinal diseases, eye diseases, cancers, rheumatic diseases, demyelinating diseases and fibrotic diseases.

Viewed from a fifth aspect, there is provided a compound as defined in the second aspect, or a pharmaceutical composition as defined in the second aspect, for use in the inhibition of Bromodomain and Extra-Terminal proteins.

Viewed from a sixth aspect, there is provided a method for the treatment or prophylaxis of inflammatory skin disorders, respiratory diseases, gastrointestinal diseases eye diseases cancers, rheumatic diseases, demyelinating diseases and fibrotic diseases, said method comprising administering to a subject, an effective amount of a compound as defined in the first aspect, or a pharmaceutical composition as defined in the second aspect.

Viewed from a seventh aspect, there is provided a method of inhibiting Bromodomain and Extra-Terminal protein activity in a subject, said method comprising administering to a subject an effective amount of a compound as defined in the first aspect, or a pharmaceutical composition as defined in the second aspect.

### DETAILED DESCRIPTION OF THE INVENTION

Structurally novel derivatives of N-methyl-2-pyridone have been found to be surprisingly effective in inhibiting all four BET BRDs to at least a similar degree as the inhibitors known in the art. In some cases, known BET protein inhibitors are outperformed by the compounds described herein. The compounds are now described in detail.

In the discussion that follows, reference is made to a number of terms, which have the meanings provided below, unless a context indicates to the contrary. The nomenclature used herein for defining compounds, in particular the compounds according to the invention, is in general based on the rules of the IUPAC organisation for chemical compounds, specifically the “IUPAC Compendium of Chemical Terminology (Gold Book)”. For the avoidance of doubt, if a rule of the IUPAC organisation is contrary to a definition provided herein, the definition herein is to prevail. Furthermore, if a compound structure is contrary to the name provided for the structure, the structure is to prevail.

The term “therapeutic index”, also known as the “therapeutic window” or “safety window” defines the relative safety of a drug. The therapeutic index may be calculated as the ratio of the area under the curve (AUC) in blood, at a concentration of drug that results in no toxicity (No Observed Adverse Effect Level—NOAEL), to the concentration of drug that produces the desired efficacy, typically the dose that has a 50% effect—the Effective dose 50 or ED50. TI=AUC(NOAEL)/AUC(ED50). A drug with a higher therapeutic index is preferable, since administration of the drug is less likely to lead to unwanted side effects, and more drug may be administered to treat a subject more effectively. The efficacy of BET inhibitors is driven by their inhibition of the function of BDII, whereas inhibition of the function of BDI leads to unwanted side effects. Thus, drugs that selectively inhibit the function of BDII over BDI have the potential to modulate gene expression and treat diseases that are at least in part caused by abnormal regulation of BET and are less likely to give rise to unwanted side effects with respect to pan inhibitors administered at the same dose. A higher dose of drugs that selectively inhibit BDII over BDI may be administered with respect to pan inhibitors, thus such selective drugs may be more efficacious.

The term “aromatic” defines a cyclically conjugated molecular entity with a stability (due to delocalisation) significantly greater than that of a hypothetical localised structure. The Hückel rule is often used in the art to assess aromatic character; monocyclic planar (or almost planar) systems of trigonally (or sometimes diagonally) hybridised atoms that contain (4n+2) π-electrons (where n is a non-negative integer) will exhibit aromatic character. The rule is generally limited to n=0 to 5.

The term “heteroaromatic” defines a cyclically conjugated molecular entity comprising heteroatoms, with a stability (due to delocalisation) significantly greater than that of a hypothetical localised structure.

The term “cyclic” or variants thereof defines a compound in which one or more series of atoms in the compound is connected to form a ring. Whereas, the term “acyclic” defines a compound containing no rings of atoms.

The term “conjugated” or variants thereof defines a molecular entity whose structure may be represented as a system of alternating single and multiple bonds. In such systems, conjugation is the interaction of one p-orbital with another across an intervening TT-bond in such structures. In appropriate molecular entities d-orbitals may be involved. The term is also extended to the analogous interaction involving a p-orbital containing an unshared electron pair.

The term “delocalised” defines the TT-bonding in a conjugated system where the bonding is not localised between two atoms, but instead each link has a fractional double bond character, or bond order.

The term “comprising” or variants thereof will be understood to imply the inclusion of a stated element, integer or step, or group of elements, integers or steps, but not the exclusion of any other element, integer or step, or group of elements, integers or steps.

The term “consisting” or variants thereof will be understood to imply the inclusion of a stated element, integer or step, or group of elements, integers or steps, and the exclusion of any other element, integer or step or group of elements, integers or steps.

The term “alkyl” is well known in the art and defines univalent groups derived from alkanes by removal of a hydrogen atom from any carbon atom, wherein the term “alkane” is intended to define cyclic or acyclic branched or unbranched hydrocarbons having the general formula CₙH₂ₙ₊₂, wherein n is an integer ≥1.

The term “cycloalkyl” defines all univalent groups derived from cycloalkanes by removal of a hydrogen atom from a ring carbon atom. The term “cycloalkane” defines saturated monocyclic and polycyclic hydrocarbons.

The term “alkylol” defines a hydroxy derivative of an alkyl radical, i.e. a hydroxy-alkyl.

The term “halo” is well known in the art and defines a halogen radical that, when bonded to a carbon radical makes a fluoride, chloride, bromide or iodide compound.

The term “alkyloxy” is synonymous with “alkoxy” and when used herein defines a univalent group comprising an alkyl singly bonded to an oxygen atom, derived from the corresponding alcohol by removal of the hydrogen atom bonded to the oxygen atom.

The term “alkylamino” is synonymous with “alkamino” and when used herein defines a univalent group comprising an alkyl singly bonded to an amino group, derived from the corresponding amine by removal of a hydrogen atom bonded to the nitrogen atom.

The term “oxacycloalkyl” defines a univalent group comprising a cycloalkyl, in which one of the CH₂ moieties is replaced with an oxide. Similarly, the term “azacycloalkyl” defines a univalent group comprising a cycloalkyl, in which one of the CH₂ moieties is replaced with an NH moiety.

The term “treatment” defines the therapeutic treatment of a human or non-human animal, in order to impede or reduce or halt the rate of the progress of the condition, or to ameliorate or cure the condition. Prophylaxis of the condition as a result of treatment is also included. References to prophylaxis are intended herein not to require complete prevention of a condition: its development may instead be hindered through treatment in accordance with the invention. Typically, treatment is not prophylactic, and the compound or composition is administered to a patient having a diagnosed or suspected condition. By an “effective amount” herein defines an amount of the compound or composition of the invention that is sufficient to impede the noted diseases and thus produces the desired therapeutic or inhibitory effect.

The term “stereoisomer” is used herein to refer to isomers that possess identical molecular formulae and sequence of bonded atoms, but which differ in the arrangement of their atoms in space.

The term “enantiomer” defines one of a pair of molecular entities that are mirror images of each other and non-superimposable, i.e. cannot be brought into coincidence by translation and rigid rotation transformations. Enantiomers are chiral molecules, i.e. are distinguishable from their mirror image.

The term “racemic” is used herein to pertain to a racemate. A racemate defines a substantially equimolar mixture of a pair of enantiomers.

The term “diastereoisomers” (also known as diastereomers) defines stereoisomers that are not related as mirror images.

The term “solvate” is used herein to refer to a complex comprising a solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

The term “isotope” is used herein to define a variant of a particular chemical element, in which the nucleus necessarily has the same atomic number but has a different mass number owing to it possessing a different number of neutrons.

The term “prodrug” is used herein to refer to a compound which acts as a drug precursor and which, upon administration to a subject, undergoes conversion by metabolic or other chemical processes to yield a compound of formula (I).

The term “pharmaceutically acceptable excipient” defines substances other than a pharmacologically active drug or prodrug, which are included in a pharmaceutical product.

The term “topical” when used with respect to compounds or compositions of the invention is used to refer to the ability to apply the compound or composition to body surfaces, for example skin or mucous membranes. Topical compounds or compositions may be applied in the form of creams, foams, gels, lotions or ointments.

The term “oral” when used with respect to compounds or compositions of the invention is used to refer to the ability to administer the compound or composition through the mouth. Typically, oral compounds exhibit a systemic effect rather than a topical effect, i.e. they affect multiple organ systems, rather than a local area.

The terms “transduce” or “transducing”, when used with respect to a signal, are synonymous with “transfer” or “transferring”, i.e. “signal transduction” is the process of transferring a signal throughout an organism, for example through a cell.

The term “pan” is used herein to refer to “all”. For example, pan inhibition of the BET family means that all of the members of the BET family (BRD2, BRD3, BRD4 and BRDT) are inhibited.

The term “T-cell” (also known as a T lymphocyte) is known in the art to refer to a lymphocyte with a T-cell receptor on the cell surface (a molecule that is responsible for recognising fragments of antigen peptides).

The term “cytokine” is used herein to refer to a small protein (~5 to 20 kDa) that is important in cell signalling, such as autocrine, paracrine and endocrine signalling, as immunomodulating agents.

The term “chemokine” is used herein to refer to a family of cytokines that are able to induce directed chemotaxis in responsive cells, i.e. they act as a chemoattractant to guide the migration of cells.

The term “intrinsic clearance” is well known in the art and refers to the ability of the liver to remove a drug in the absence of flow limitations and binding to cells or proteins in the blood. Intrinsic clearance is herein expressed as a percentage of liver blood flow, i.e.:

$$Intrinsic clearance ( \% ) = \frac{rate of drug clearance}{rate of liver blood flow} \times 100$$

The term “soft drug” refers to compounds that are rapidly metabolised on reaching the blood or liver. Highly cleared compounds are considered to have clearance rates of >70% of liver blood flow, most often clearance rates of >75%, with intermediate rates being 30-70%, most often 50-75%, and low rates being <30%, most often <50%. Soft drugs are often characterised by a predictable and controllable in vivo metabolism to non-toxic products after they have achieved their therapeutic role. Soft drugs have lower systemic exposure and may lead to a lower risk of side effects.

The systemic inhibition of drug targets is often associated with dose limiting side-effects and there is an unmet need for efficacious agents, which are well tolerated in patients. Compounds that are rapidly cleared upon entering the blood stream have lower systemic exposure and may lead to a lower risk of side effects (see Atkinson AJ Jr. and Kushner W., Annu. Rev. Pharmacol. Toxicol., 1979, 19, 105-127 and Rowland M. and Tozer T. N., Clinical Pharmacokinetics. Concepts and Applications. Lippincott Williams & Wilkins, 1995, 161-167).

It is unpredictable which groups within a drug structure will lead to rapid systemic clearance of a drug. Phenol groups, in some cases, are observed to be cleared via phase II conjugative clearance mechanisms such as glucuronidation and sulfation (see Pathways of Biotransformation—Phase II Reactions. In: Ionescu C., Caira M. R. (eds) Drug Metabolism. Springer, Dordrecht, 2005).

As alluded to above, the first aspect provides a compound of formula (I):

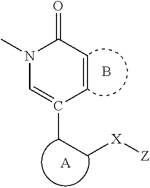

wherein ring structure A is a 5- or 6-membered aromatic or heteroaromatic ring, optionally substituted at one or more carbon and/or heteroatoms with a first substituent;
wherein each first substituent is independently selected from the group consisting of hydroxy, oxo, C₁-C₆alkyl, C₃-C₆cycloalkyl, C₁-C₆alkylol, halo, SO₂C₁-C₄alkyl, NHSO₂C₁-C₄alkyl, SO₂C₃-C₆cycloalkyl, NHSO₂C₃-C₆cycloalkyl, SO₂C₁-C₄alkylol, NHSO₂C₁-C₄alkylol, C₁-C₅alkyloxy, C₁-C₅alkylamino, SO₂NH₂, CONH₂, CONHC₁-C₄alkyl, NHCOC₁-C₄alkyl, NHSO₂N(C₁-C₄alkyl)₂, C₁-C₆fluoroalkyl, SO₂C₁-C₄fluoroalkyl, NHSO₂C₁-C₄fluoroalkyl, C₁-C₅fluoroalkyloxy, and C₁-C₅fluoroalkylamino;
X is O, CR₂, NR′ or S, wherein R is individually selected from the group consisting of H, C₁-C₄alkyl and halo, and R′ is selected from the group consisting of C₁-C₄alkyl and H;
Z is a 5- or 6-membered aromatic or heteroaromatic ring, C₁-C₆alkyl, C₃-C₆cycloalkyl, CR^{A}R^{B}R^{C}, C₂-C₅oxacycloalkyl, C₂-C₅azacycloalkyl or morpholinyl, optionally substituted at one or more carbon and/or heteroatoms with a second substituent;
wherein R^{A} is a C₃-C₅cycloalkyl, R^{B} is a C₃-C₅cycloalkyl, methyl or ethyl, and R^{C} is OH; and
each second substituent is independently selected from the group consisting of hydroxy, C₁-C₆alkyl, C₃-C₆cycloalkyl, halo, C₁-C₅alkyloxy, C₁-C₅alkylamino, oxo, cyano, C₁-C₆fluoroalkyl, C₁-C₅fluoroalkyloxy, and C₁-C₅fluoroalkylamino;
ring structure B is optionally present; wherein when ring structure B is present, it is an optionally substituted pyrrole bonded such that C is in the 4 position relative to NH; wherein the pyrrole is optionally substituted at position 2 with a third substituent;
wherein the third substituent is selected from the group consisting of CONHC₁-C₄alkyl, CONH₂, CONHC₁-C₆fluoroalkyl, CONHC₃-C₆cycloalkyl optionally substituted at one or more carbon atoms with a methyl or ethyl; CONHC₃-C₅cyclofluoroalkyl optionally substituted at one or more carbon atoms with a methyl or ethyl, NHCOC₁-C₄alkyl and NHCOC₁-C₄fluoroalkyl;
with the proviso that when A is 6-membered, it is substituted at least once with a hydroxy or oxo group.

B is optionally present. When absent, the carbon atoms positioned ortho and meta to C are each bound to H. When present, ring structure B is an optionally substituted pyrrole; C is in the 4 position relative to NH, thereby forming a 6-methyl-1,6-dihydro-7H-pyrrolo[2,3-c]pyridin-7-one derivative.

The pyrrole is optionally substituted at position 2 with a third substituent, which is selected from the group consisting of CONHC₁-C₄alkyl, CONH₂, CONHC₁-C₆fluoroalkyl, CONHC₃-C₆cycloalkyl optionally substituted at one or more carbon atoms with a methyl or ethyl; CONHC₃-C₅cyclofluoroalkyl optionally substituted at one or more carbon atoms with a methyl or ethyl, NHCOC₁-C₄alkyl and NHCOC₁-C₄fluoroalkyl. The CONHC₃-C₆cycloalkyl may be unsubstituted. Often, the third substituent is selected from the group consisting of CONHC₁-C₄alkyl, CONHC₁-C₆fluoroalkyl, CONHC₃-C₆cycloalkyl optionally substituted at one or more carbon atoms with a methyl or ethyl, and CONHC₃-C₅cyclofluoroalkyl optionally substituted at one or more carbon atoms with a methyl or ethyl. The third substituent may be selected from the group consisting of CONHC₁-C₄alkyl, CONHC₁-C₆fluoroalkyl, CONHC₃-C₆cycloalkyl and CONHC₃-C₅cyclofluoroalkyl optionally substituted at one or more carbon atoms with a methyl or ethyl. The third substituent may be selected from the group consisting of CONHC₁-C₄alkyl, CONH₂, CONHC₁-C₆fluoroalkyl, CONHC₃-C₅cycloalkyl; CONHC₃-C₅cyclofluoroalkyl, NHCOC₁-C₄alkyl and NHCOC₁-C₄fluoroalkyl. Often, the third substituent is CONHC₁-C₄alkyl, typically CONHethyl. Typically the pyrrole is unsubstituted. Most typically, the pyrrole is unsubstituted or is substituted at position 2 with CONHethyl.

Typically, B is present and is sometimes a pyrrole optionally substituted at position 2 with a third substituent that is a CONHC₁-C₄alkyl. Often the third substituent is CONHethyl. Typically, B is present and is an unsubstituted pyrrole. Most typically, B is present and is an unsubstituted pyrrole or a pyrrole substituted at position 2 with CONHethyl. Therefore, the compound of the invention is typically represented by formula (II) or (III):

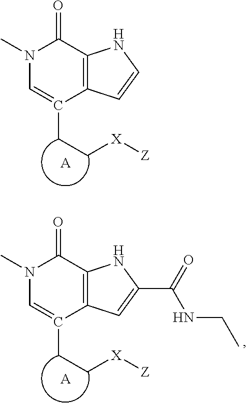

wherein A, X and Z are as defined for formula (I), with the proviso that when A is 6-membered, it is substituted at least once with a hydroxy or oxo group.

Often, when A is 6-membered, it is substituted at least once with a hydroxy group positioned ortho or meta to X, or an oxo group.

A connects C to X and may be any 5-membered aromatic or heteroaromatic ring, or any 6-membered aromatic or heteroaromatic ring that is substituted at least once with a hydroxy or oxo group. 5-membered aromatic or heteroaromatic rings include thiazole, oxazole, imidazole, isoxazole, pyrazole, thiophene, pyrrole, furan and cyclopentadienyl. 5-membered heteroaromatic rings also include triazole, such as 1,2,4-triazole. 6-membered aromatic or heteroaromatic rings include benzene, pyridine, pyridone, pyrazine, pyrimidine, pyridazine, 1,2,3-triazine, 1,2,4-triazine and 1,3,5-triazine. The 5-membered or 6-membered aromatic or heteroaromatic rings may be substituted at one or more carbon and/or heteroatoms with the first substituent. For example, when A is a pyridine ring, it may be substituted with the first substituent at any of the one, two or three carbon atoms that are not bound to C or X and/or at the nitrogen atom.

When A is 5-membered, it is often unsubstituted or substituted at one position. Typically, when A is 5-membered, it is unsubstituted.

Often, A is selected from the group consisting of benzene, pyridine, thiazole, pyridone, pyrazole, imidazole and triazole, optionally substituted at one or more carbon and/or heteroatoms with the first substituent. Typically, A is selected from the group consisting of benzene, pyridine, thiazole and pyridone, optionally substituted at one or more carbon and/or heteroatoms with the first substituent. When A is a pyridone, it may be a 2-, 3- or 4-pyridone. Commonly, when A is a pyridone, it is a 2-pyridone, i.e. A is commonly selected from the group consisting of benzene, pyridine, thiazole and 2-pyridone, optionally substituted at one or more carbon or heteroatoms with the first substituent.

When A is a thiazole, it is commonly bound to C via the thiazole carbon atom at position 5, and bound to X via the thiazole carbon at position 4. The resulting C-A-X moiety is represented by:

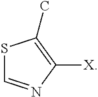

The thiazole may be substituted at one or more carbon and/or nitrogen atoms with the first substituent. Often, the thiazole is substituted at position 2 with the first substituent.

When A is a 2-pyridone, it is typically either: bound to C via the carbon atom at position 4, and bound to X via the carbon atom at position 3, or bound to C via the carbon atom at position 4 and bound to X via the carbon atom at position 5. The resulting C-A-X moieties are represented by:

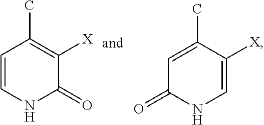

respectively.

The 2-pyridone may be substituted at one or more carbon and/or nitrogen atoms with the first substituent. Often, the 2-pyridone is substituted at one or more carbon and/or nitrogen atoms with a C₁-C₆alkyl. Commonly, the C₁-C₆alkyl is a C₁-C₄alkyl selected from the group consisting of methyl, ethyl, n-propyl, isopropyl, n-butyl, sec-butyl, isobutyl and tert-butyl. Typically, the C₁-C₆alkyl is a methyl. Often, the 2-pyridone is substituted at the nitrogen atom with a methyl.

When A is a pyrazole, it is commonly bound to C via the pyrazole carbon atom at position 5, and bound to X by the nitrogen atom at position 1. The resulting C-A-X moiety is represented by:

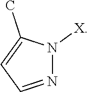

The pyrazole may be substituted at one or more carbon and/or nitrogen atoms with the first substituent. Often, the pyrazole is substituted at position 3 or 4 with the first substituent.

When A is an imidazole, it is commonly bound to C via the imidazole carbon atom at position 2, and bound to X by the nitrogen atom at position 1. The resulting C-A-X moiety is represented by:

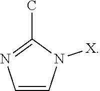

The imidazole may be substituted at one or more carbon and/or nitrogen atoms with the first substituent. Often, the imidazole is substituted at position 4 or 5 with the first substituent.

When A is a triazole, it is typically a 1,2,4-triazole. When A is a 1,2,4-triazole it is commonly bound to C via the imidazole carbon atom at position 5, and bound to X by the nitrogen atom at position 1. The resulting C-A-X moiety is represented by:

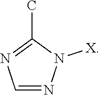

The 1,2,4-triazole may be substituted at one or more carbon and/or nitrogen atoms with the first substituent. Often, the 1,2,4-triazole is substituted at position 3 with the first substituent.

The first substituent may be hydroxy, oxo, C₁-C₆alkyl, C₃-C₆cycloalkyl, C₁-C₆alkylol, halo, SO₂C₁-C₄alkyl, NHSO₂C₁-C₄alkyl, SO₂C₃-C₆cycloalkyl, NHSO₂C₃-C₆cycloalkyl, SO₂C₁-C₄alkylol, NHSO₂C₁-C₄alkylol, C₁-C₅alkyloxy, C₁-C₅alkylamino, SO₂NH₂, CONH₂, CONHC₁-C₄alkyl, NHCOC₁-C₄alkyl, NHSO₂N(C₁-C₄alkyl)₂, C₁-C₆fluoroalkyl, SO₂C₁-C₄fluoroalkyl, NHSO₂C₁-C₄fluoroalkyl, C₁-C₅fluoroalkyloxy, and/or C₁-C₅fluoroalkylamino. When the first substituent is selected from SO₂C₁-C₄alkyl, NHSO₂C₁-C₄alkyl, SO₂C₁-C₄fluoroalkyl and NHSO₂C₁-C₄fluoroalkyl, it is often SO₂CH₃, NHSO₂CH₃, SO₂CF₃ and/or NHSO₂CF₃, i.e. methanesulfonyl, methanesulfonamido, trifluoromethanesulfonyl and/or trifluoromethanesulfonamido. When the first substituent is selected from SO₂C₃-C₆cycloalkyl and NHSO₂C₃-C₆cycloalkyl, it is often SO₂C₃H₅, SO₂C₅H₉, SO₂C₆H₁₁, NHSO₂C₃H₅, NHSO₂C₅Hg and/or NHSO₂C₆H₁₁, i.e. cyclopropanesulfonyl, cyclopentanesulfonyl, cyclohexanesulfonyl, cyclopropanesulfonamido, cyclopentanesulfonamido and/or cyclohexanesulfonamido. When the first substituent is selected from SO₂C₁-C₄alkylol and NHSO₂C₁-C₄alkylol, it is often SO₂C(CH₃)₂OH and/or NHSO₂C(CH₃)₂OH, i.e. tert-butanolsulfonyl and/or tert-butanolsulfonamido.

Therefore, each first substituent is often independently selected from the group consisting of hydroxy, oxo, C₁-C₆alkyl, C₃-C₆cycloalkyl, C₁-C₆alkylol, halo, SO₂CH₃, NHSO₂CH₃, SO₂^{t}Bu, NHSO₂^{t}Bu, SO₂C₃H₅, SO₂C₅H₉, SO₂C₆H₁₁, NHSO₂C₃H₅, NHSO₂C₅H₉, NHSO₂C₆H₁₁, C₁-C₅alkyloxy, C₁-C₅alkylamino, SO₂NH₂, CONH₂, CONHC₁-C₄alkyl, NHCOC₁-C₄alkyl, NHSO₂N(C₁-C₄alkyl)₂, C₁-C₆fluoroalkyl, SO₂CF₃, NHSO₂CF₃, C₁-C₅fluoroalkyloxy, and/or C₁-C₅fluoroalkylamino.

Typically, each first substituent is independently selected from the group consisting of hydroxy, oxo, C₁-C₆alkyl, C₁-C₆alkylol, C₃-C₆cycloalkyl, halo, SO₂CH₃, NHSO₂CH₃, SO₂C₃H₅, SO₂C₅H₉, SO₂C₆H₁₁, NHSO₂C₃H₅, NHSO₂C₅H₉ and NHSO₂C₆H₁₁. Often, each first substituent is independently selected from the group consisting of hydroxy, oxo, C₁-C₆alkyl, C₃-C₆cycloalkyl, halo, SO₂CH₃ and NHSO₂CH₃.

Commonly, each first substituent is independently selected from the group consisting of hydroxy, oxo, C₁-C₅alkyl, C₃-C₆cycloalkyl, C₁-C₆alkylol, and halo. Typically, the C₁-C₆alkyl is a C₁-C₄alkyl selected from the group consisting of methyl, ethyl, n-propyl, isopropyl, n-butyl, sec-butyl, isobutyl and tert-butyl, and the C₃-C₆cycloalkyl is selected from the group consisting of cyclopropyl, cyclopentyl and cyclohexyl. Typically, the C₁-C₆alkylol is hydroxymethyl, hydroxyethyl, hydroxy-n-propyl, hydroxy-isopropyl, hydroxy-n-butyl, hydroxy-sec-butyl, hydroxy-isobutyl and hydroxy-tert-butyl. Typically, the halo is fluoro or chloro. Therefore, each first substituent is commonly independently selected from the group consisting of hydroxy, oxo, methyl, ethyl, n-propyl, isopropyl, n-butyl, sec-butyl, isobutyl, tert-butyl, cyclopropyl, cyclopentyl, cyclohexyl, hydroxymethyl, hydroxyethyl, hydroxy-n-propyl, hydroxy-isopropyl, hydroxy-n-butyl, hydroxy-sec-butyl, hydroxy-isobutyl and hydroxy-tert-butyl, fluoro and chloro.

Most typically, each first substituent is independently selected from the group consisting of hydroxy, oxo, methyl, ethyl, isopropyl, tert-butyl, cyclopropyl, hydroxy-tert-butyl, fluoro and chloro.

Often, each first substituent is independently selected from the group consisting of hydroxy, oxo, methyl and halo.

When A is benzene or pyridine, it is substituted at least once with a hydroxy group. Sometimes, it is substituted with a hydroxy group and a further first substituent, selected from the group consisting of methyl, ethyl, n-propyl, isopropyl, n-butyl, sec-butyl, isobutyl, tert-butyl, cyclopropyl, cyclopentyl, cyclohexyl, fluoro and chloro. Typically, it is substituted with a hydroxy group and a further first substituent, selected from the group consisting of methyl, fluoro and chloro. Often, the at least one hydroxy group is positioned ortho or meta to X.

X is O, CR₂ or NR′, wherein R is individually selected from the group consisting of H, C₁-C₄alkyl and halo, and R′ is selected from the group consisting of C₁-C₄alkyl and H.

When X is CR₂, halo is typically chloro or fluoro. Therefore, R is typically individually selected from the group consisting of H, C₁-C₄alkyl, fluoro and chloro.

When X is CR₂ or NR′, C₁-C₄alkyl may be methyl, ethyl, n-propyl, isopropyl, n-butyl, sec-butyl, isobutyl or tert-butyl. Therefore, R is typically individually selected from the group consisting of H, methyl, ethyl, n-propyl, isopropyl, n-butyl, sec-butyl, isobutyl or tert-butyl, fluoro and chloro, and R′ is selected from the group consisting of H, methyl, ethyl, n-propyl, isopropyl, n-butyl, sec-butyl, isobutyl or tert-butyl and H. Sometimes, R is individually selected from the group consisting of H, methyl and halo, and R′ is selected from the group consisting of methyl and H. Often, R is individually selected from the group consisting of H, methyl and fluoro, and R′ is methyl.

Typically, X is O, i.e. A is bound to Z via an oxide.

Z is a 5- or 6-membered aromatic or heteroaromatic ring, C₁-C₆alkyl, C₃-C₆cycloalkyl, CR^{A}R^{B}R^{C}, C₂-C₅oxacycloalkyl, C₂-C₅azacycloalkyl or morpholinyl, optionally substituted at one or more carbon and/or heteroatoms with a second substituent, each selected independently from the group consisting of hydroxy, C₁-C₆alkyl, C₃-C₆cycloalkyl, halo, C₁-C₅alkyloxy, C₁-C₅alkylamino, oxo, cyano, C₁-C₆fluoroalkyl, C₁-C₅fluoroalkyloxy, and C₁-C₅fluoroalkylamino;
wherein R^{A} is a C₃-C₅cycloalkyl, R^{B} is a C₃-C₅cycloalkyl, methyl or ethyl, and R^{C} is OH.
Z may be any optionally substituted 5-membered aromatic or heteroaromatic ring, for example Z may be thiazole, oxazole, imidazole, isoxazole, pyrazole, thiophene, pyrrole, furan or cyclopentadienyl.

Alternatively, Z may be any optionally substituted 6-membered aromatic or heteroaromatic ring, for example Z may be benzene, pyridine, pyridone, pyrazine, pyrimidine, pyridazine, 1,2,3-triazine, 1,2,4-triazine or 1,3,5-triazine.

Otherwise, Z may be an optionally substituted C₁-C₆alkyl, C₃-C₆cycloalkyl, CR^{A}R^{B}R^{C}, C₂-C₅oxacycloalkyl, C₂-C₅azacycloalkyl or morpholinyl. Typically, the C₁-C₆alkyl is a C₁-C₄alkyl selected from the group consisting of methyl, ethyl, n-propyl, isopropyl, n-butyl, sec-butyl, isobutyl and tert-butyl; the C₃-C₆cycloalkyl is selected from the group consisting of cyclopropyl, cyclopentyl and cyclohexyl; R^{A} is cyclopropyl, cyclobutyl or cyclopentyl, R^{B} is cyclopropyl, cyclobutyl, cyclopentyl, methyl or ethyl; the C₂-C₅oxacycloalkyl is selected from the group consisting of oxacyclopropyl, oxacyclopentyl and oxacyclohexyl; and the C₂-C₅azacycloalkyl is selected from the group consisting of azacyclopropyl, azacyclopentyl and azacyclohexyl.

Often, Z is selected from the group consisting of benzene, pyridine, thiazole, pyridone, methyl, ethyl, n-propyl, isopropyl, n-butyl, tert-butyl, cyclopropyl, cyclopentyl, cyclohexyl, oxacyclopentyl, oxacyclohexyl, azacyclopentyl, azacyclohexyl and morpholinyl, optionally substituted at one or more carbon and/or heteroatoms with a second substituent. Sometimes, Z is selected from the group consisting of benzene, pyridine, pyridone, n-propyl, isopropyl, n-butyl, tert-butyl, cyclopropyl, cyclopentyl and cyclohexyl, optionally substituted at one or more carbon and/or nitrogen atoms with a second substituent.

Z is commonly an optionally substituted 6-membered aromatic or heteroaromatic ring, a C₁-C₆alkyl, or a C₃-C₆cycloalkyl.

Typically, Z is a phenyl or pyridyl ring, a C₁-C₆alkyl, or a C₃-C₆cycloalkyl optionally substituted at one or more carbon and/or nitrogen atoms with a second substituent.

Each second substituent is independently selected from the group consisting of hydroxy, C₁-C₆alkyl, C₃-C₆cycloalkyl, halo, C₁-C₅alkyloxy, C₁-C₅alkylamino, oxo, cyano, C₁-C₆fluoroalkyl, C₁-C₅fluoroalkyloxy, and C₁-C₅fluoroalkylamino.

Often, each second substituent is independently selected from the group consisting of hydroxy, C₁-C₆alkyl, C₃-C₆cycloalkyl, and halo. Typically, the C₁-C₆alkyl is a C₁-C₄alkyl selected from the group consisting of methyl, ethyl, n-propyl, isopropyl, n-butyl, sec-butyl, isobutyl and tert-butyl, and the C₃-C₆cycloalkyl is selected from the group consisting of cyclopropyl, cyclopentyl and cyclohexyl. Often, the halo is fluoro, chloro or bromo. Therefore, each second substituent is often independently selected from the group consisting of hydroxy, methyl, ethyl, n-propyl, isopropyl, n-butyl, sec-butyl, isobutyl, tert-butyl, cyclopropyl, cyclopentyl, cyclohexyl, fluoro, chloro and bromo. Typically, the halo is fluoro or chloro. Therefore, each second substituent is commonly independently selected from the group consisting of hydroxy, methyl, ethyl, n-propyl, isopropyl, n-butyl, sec-butyl, isobutyl, tert-butyl, cyclopropyl, cyclopentyl, cyclohexyl, fluoro and chloro.

Sometimes, each second substituent is independently selected from the group consisting of hydroxy, methyl, ethyl, isopropyl, tert-butyl, fluoro, chloro and bromo. Often, each second substituent is independently selected from the group consisting of hydroxy, methyl, ethyl, isopropyl, tert-butyl, fluoro and chloro. Typically, each second substituent is independently selected from the group consisting of hydroxy, methyl, fluoro and chloro. For example, Z may be a phenyl ring substituted by two methyl groups positioned ortho to X, and further substituted by a fluoro positioned para to X. Typically, each second substituent is selected from any one or a combination of hydroxy, methyl or fluoro. Most typically, each second substituent is hydroxy.

Z is often a phenyl ring optionally substituted at one to three carbon atoms with a second substituent, each second substituent independently selected from the group consisting of hydroxy, methyl, ethyl, isopropyl, tert-butyl, fluoro and chloro; a pyridyl ring optionally substituted at one carbon atom with a hydroxy; a C₁-C₆alkyl; or a C₃-C₆cycloalkyl.

Sometimes, C-A-X of formula (I) is any one of formulae (Ia), (Ib), (Ic), (Id) or (Id′):

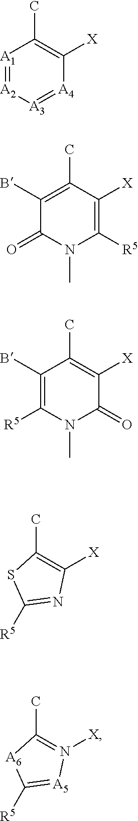

wherein A₁ is CR¹ or N, A₂ is CR² or N, A₃ is CR³ or N, A₄ is CR⁴, A₅ is CR⁵ or N and A₆ is CR⁵ or N;
R¹ is H or hydroxy;
R² is H, hydroxy, C₁-C₆alkyl, C₃-C₆cycloalkyl, halo, SO₂C₁-C₄alkyl, NHSO₂C₁-C₄alkyl, SO₂C₃-C₆cycloalkyl, NHSO₂C₃-C₆cycloalkyl, C₁-C₅alkyloxy or C₁-C₅alkylamino;
R³ and R⁴ are independently selected from the group consisting of H, hydroxy, C₁-C₆alkyl, C₃-C₆cycloalkyl, halo, C₁-C₅alkyloxy or C₁-C₅alkylamino;
with the proviso that at least one of R¹, R², R³ or R⁴ is hydroxy;
B′ is H or hydroxy; and
R⁵ is either H or the first substituent, defined above.

Sometimes, C-A-X of formula (I) is any one of formulae (Ia), (Ib), (Ic) or (Id):

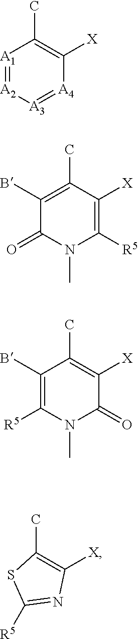

wherein A₁ is CR¹ or N, A₂ is CR² or N, A₃ is CR³ or N and A₄ is CR⁴;
R¹ is H or hydroxy;
R² is H, C₁-C₆alkyl, C₃-C₆cycloalkyl, halo, SO₂C₁-C₄alkyl, NHSO₂C₁-C₄alkyl, SO₂C₃-C₆cycloalkyl, NHSO₂C₃-C₆cycloalkyl, C₁-C₅alkyloxy or C₁-C₅alkylamino;
R³ and R⁴ are independently selected from the group consisting of H, hydroxy, C₁-C₆alkyl, C₃-C₆cycloalkyl, halo, C₁-C₅alkyloxy or C₁-C₅alkylamino;
with the proviso that at least one of R¹, R³ or R⁴ is hydroxy;
B′ is H or hydroxy; and
R⁵ is either H or the first substituent, defined above.

Typically, R² is H, C₁-C₃alkyl, halo, SO₂C₁-C₄alkyl or NHSO₂C₁-C₄alkyl; and R³ and R⁴ are independently selected from the group consisting of H, hydroxy, C₁-C₃alkyl and halo. Often, R² is H, C₁-C₃alkyl, fluoro, chloro, SO₂CH₃ or NHSO₂CH₃; and R³ and R⁴ are independently selected from the group consisting of H, hydroxy, C₁-C₃alkyl and fluoro or chloro.

Typically, R⁵ is H.

Often, when CAX is represented by formula (Ia), Z is a phenyl ring, optionally substituted at one or more carbon atoms with the second substituent; a C₁-C₆alkyl; or a C₃-C₆cycloalkyl; and when CAX is represented by any one of formulae (Ib), (Ic) and (Id), Z is a phenyl or pyridyl ring, optionally substituted at one or more carbon and/or nitrogen atoms with the second substituent; a C₁-C₆alkyl; or a C₃-C₆cycloalkyl.

Often, when CAX is represented by formula (Ia), Z is a phenyl ring, optionally substituted at one or more carbon atoms with the second substituent; a C₁-C₆alkyl; or a C₃-C₆cycloalkyl; and when CAX is represented by any one of formulae (Ib), (Ic), (Id) and (Id′), Z is a phenyl or pyridyl ring, optionally substituted at one or more carbon and/or nitrogen atoms with the second substituent; a C₁-C₆alkyl; or a C₃-C₆cycloalkyl.

Typically, when CAX is represented by formula (Ia), Z is an unsubstituted phenyl ring; and when CAX is represented by any one of formulae (Ib), (Ic) and (Id), Z is a phenyl or pyridyl ring, optionally substituted at one or more carbon and/or nitrogen atoms with the second substituent, each second substituent independently selected from the group consisting of hydroxy, methyl, fluoro and chloro.

Typically, when CAX is represented by formula (Ia), Z is an unsubstituted phenyl ring; and when CAX is represented by any one of formulae (Ib), (Ic), (Id) and (Id′), Z is a phenyl or pyridyl ring, optionally substituted at one or more carbon and/or nitrogen atoms with the second substituent, each second substituent independently selected from the group consisting of hydroxy, methyl, fluoro and chloro. Often, the compound is any one of formulae (Ie) to (Ili):

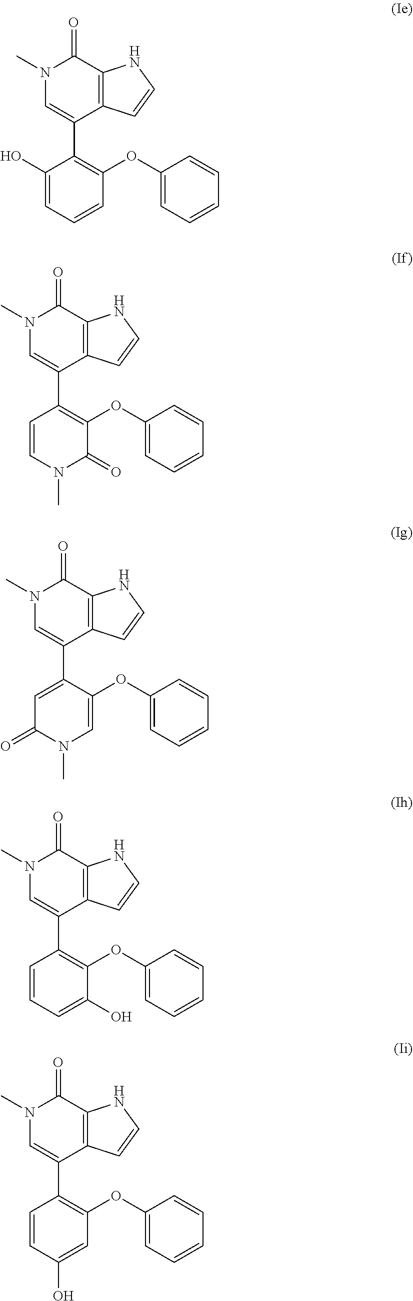

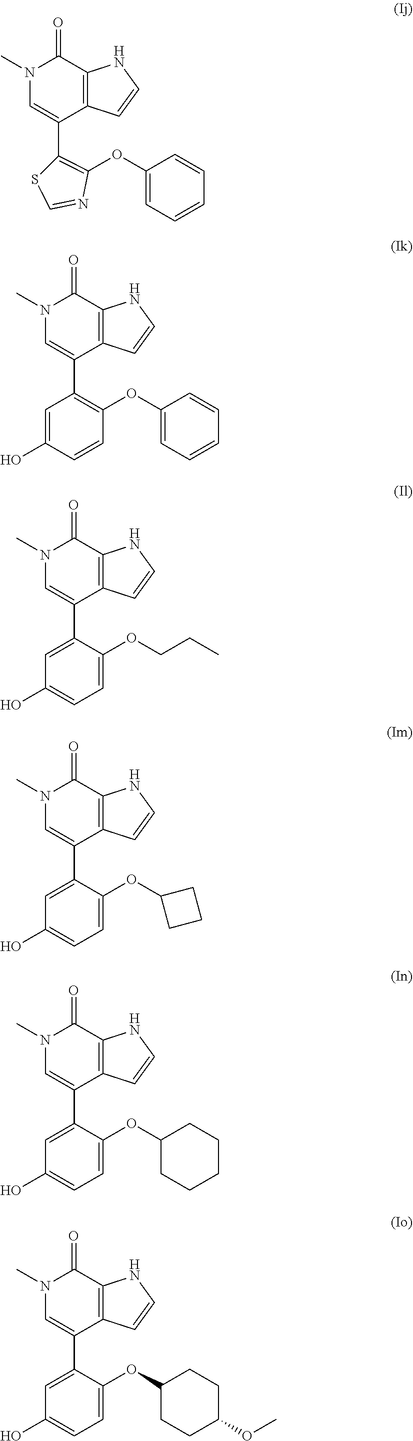

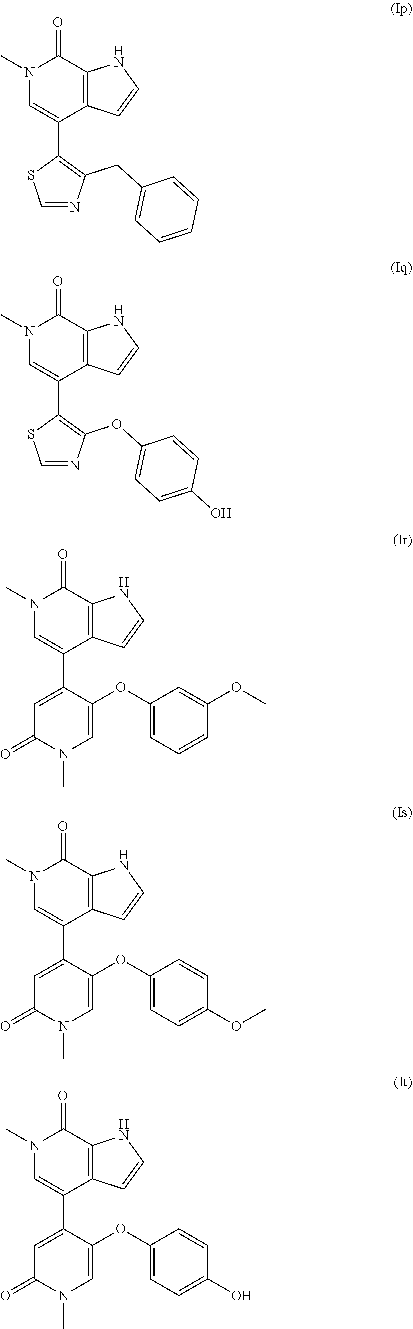

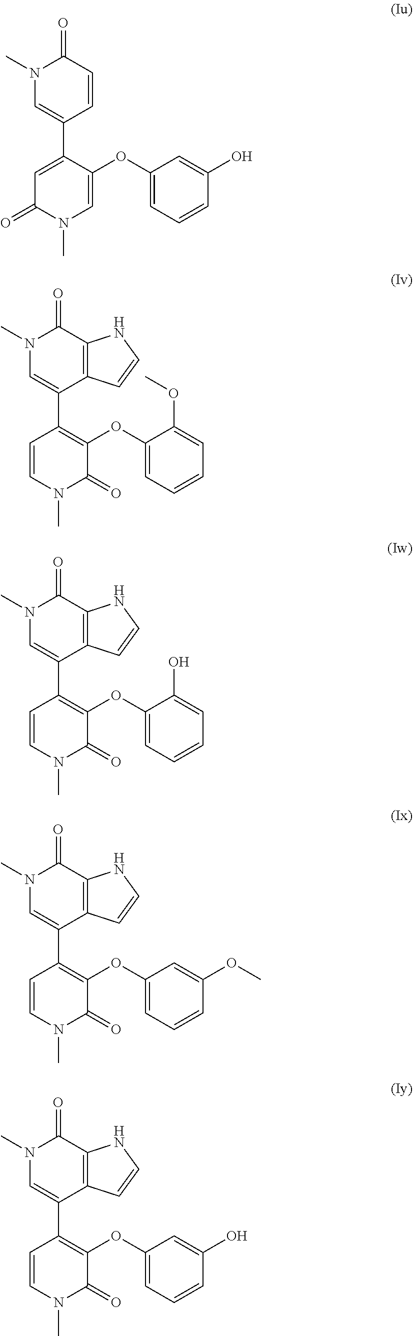

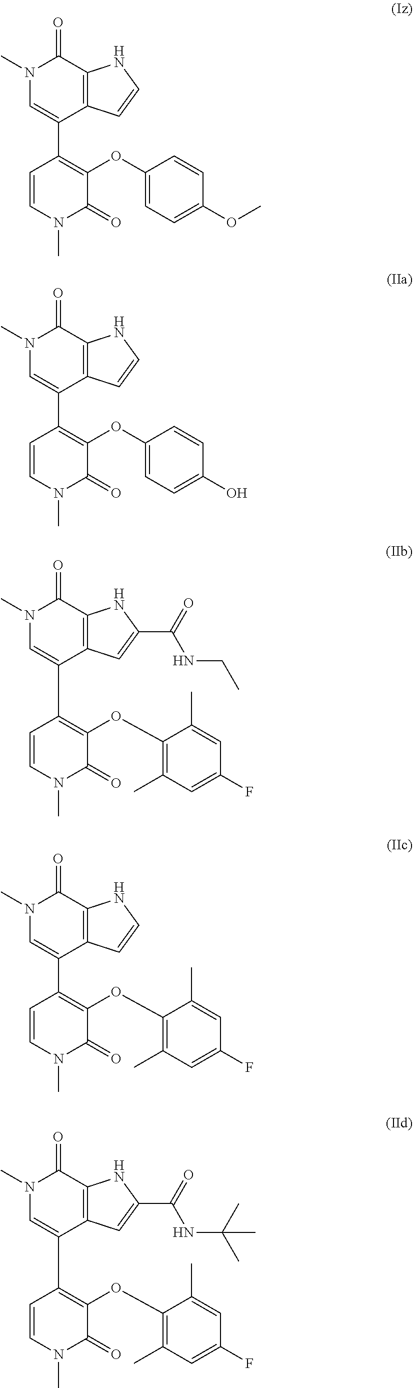

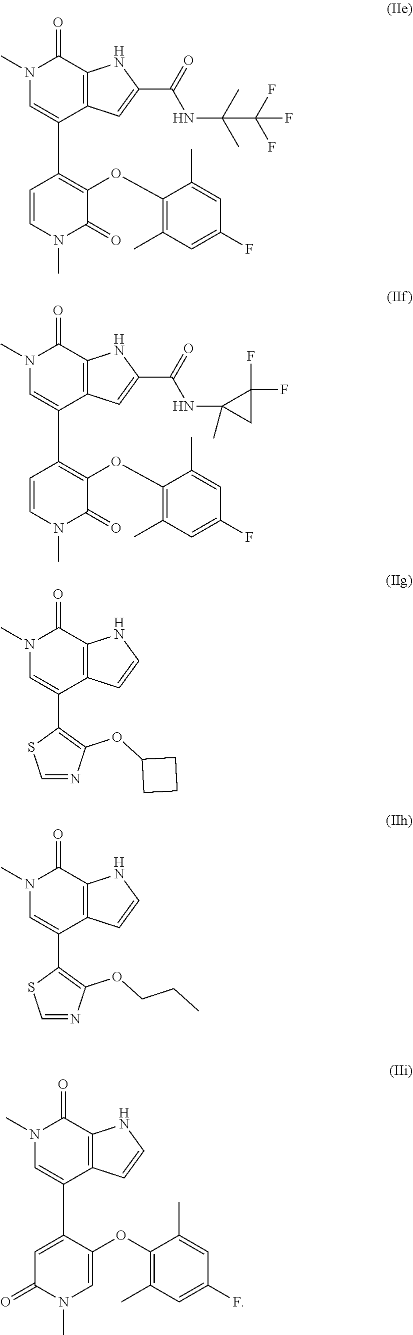

Commonly, the compound is any one of formulae (Ie), (If), (Ig), (Ih), (Ii) (Ij) or (Ik). Typically, the compound is of formula (Ih) or (IIb).

The compounds described herein may be in the form of a pharmaceutically acceptable salt. The term “pharmaceutically acceptable salt” is intended to define organic and/or inorganic salts that are pharmaceutically useful. The compounds of the invention may be isolated from reaction mixtures as pharmaceutically acceptable salts. Alternatively, the pharmaceutically acceptable salt may be prepared in situ during the final isolation and purification of compounds of the invention by reacting a carboxylic acid-containing moiety with a suitable base such as a hydroxide, carbonate or bicarbonate of a pharmaceutically acceptable metal cation, or with ammonia or a primary, secondary or tertiary amine. Pharmaceutically acceptable salts include cations based on alkali metals or alkaline earth metals such as lithium, sodium, potassium, calcium, magnesium and aluminium salts and nontoxic quaternary ammonia and amine cations including ammonium, tetramethylammonium, tetraethylammonium, methylamine, dimethylamine, trimethylamine, triethylamine, diethylamine, and ethylamine. Other examples of organic amines useful for the formation of base addition salts include ethylenediamine, ethanolamine, diethanolamine, piperidine, and piperazine.

The pharmaceutically acceptable salt may also be prepared by treatment of the compound of the invention with a suitable acid, for example, hydrogen chloride, hydrogen bromide, hydrogen iodide, sulfuric acid, phosphoric acid, acetic acid, trifluoroacetic acid, propionic acid, glycolic acid, maleic acid, malonic acid, methanesulfonic acid, fumaric acid, succinic acid, tartaric acid, citric acid, benzoic acid and ascorbic acid.

The compounds of the invention may exist in different stereoisomeric forms. All stereoisomeric forms and mixtures thereof, including enantiomers and racemic mixtures, are included within the scope of the invention. Such stereoisomeric forms include enantiomers and diastereoisomers. Individual stereoisomers of compounds of the invention, i.e., associated with less than 5%, preferably less than 2% and in particular less than 1% of the other stereoisomer, are included. Mixtures of stereoisomers in any proportion, for example a racemic mixture comprising substantially equal amounts of two enantiomers are also included within the invention.

Also included are solvates and isotopically-labelled compounds of the invention. Isotopically-labelled compounds are identical to those recited herein, but for the fact that one or more atoms are replaced by an atom having an atomic mass or mass number different from the atomic mass or mass number predominantly found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³⁶Cl, respectively.

In a further aspect, intermediates suitable for production of compounds of the invention are included. Specifically, intermediates of formulae (ia) to (ip) are included.

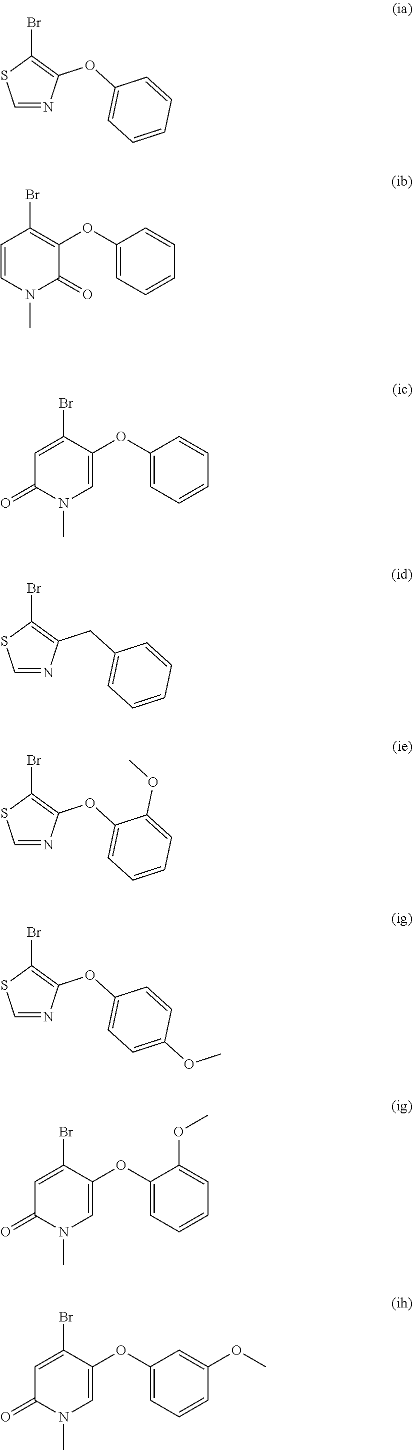

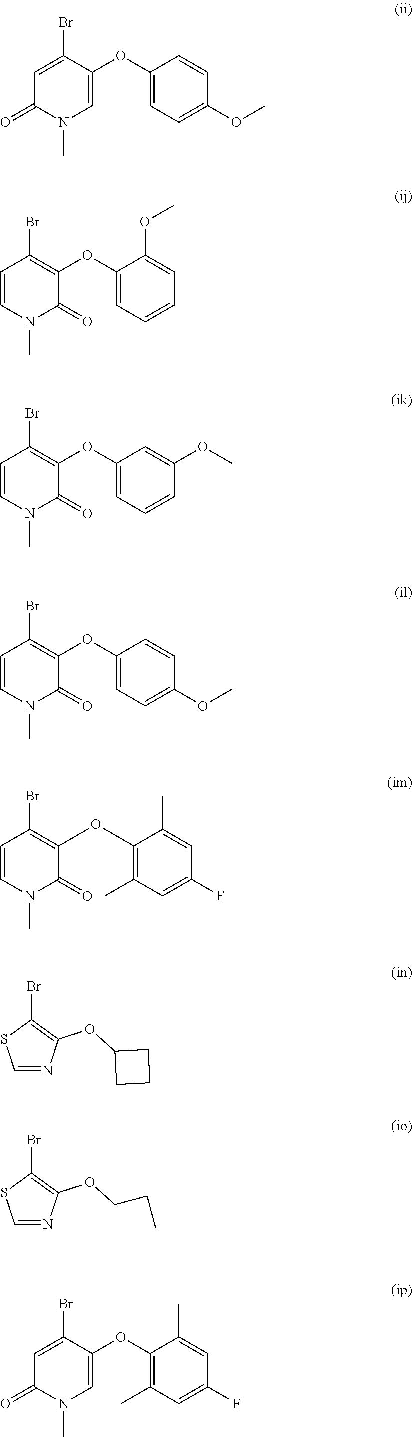

Intermediates may be of formula (ig), (ii), (ij), (ik), or (if). Often, intermediates are of formula (ia) to (id), (if), or (ih) to (ip). Typically, intermediates are of formula (im).

Prodrugs of the compounds and compositions of the invention are also within the scope of the invention. Upon administration to a subject, a prodrug undergoes conversion by metabolic or other chemical processes to yield a compound of the invention.

All amorphous and crystalline forms of the compounds of the invention are included.

Whilst it is possible for the compounds to be administered alone, it is typical to use a pharmaceutical composition. The second aspect provides a pharmaceutical composition comprising any one or a combination of the compounds defined in the first aspect, in combination with one or more pharmaceutically acceptable excipients. The excipient may aid transport of a compound to the site in the body where it is intended to act, for example by increasing the rate of dissolution of the compound into the blood stream or by increasing the stability of the compound in order to delay its release, in order to increase its efficiency and prevent damage to tender tissues. Alternatively, the excipient may be for identification purposes, or to make the compound more appealing to the patient, for example by improving its taste, smell and/or appearance. Typically, the excipient makes up the bulk of the pharmaceutical composition.

Excipients include diluents or fillers, binders, disintegrants, lubricants, colouring agents and preservatives. Diluents or fillers are inert ingredients that may affect the chemical and physical properties of the final composition. If the dosage of the compound of the invention is small then more diluents will be required to produce a composition suitable for practical use. If the dosage of the compound of the invention is high then fewer diluents will be required.

Binders add cohesiveness to powders in order to form granules, which may form a tablet. The binder must also allow the tablet to disintegrate upon ingestion so that the compound of the invention dissolves. Disintegration of the composition after administration may be facilitated through the use of a disintegrant.

An extensive overview of pharmaceutically acceptable excipients is described in the Handbook of Pharmaceutical Excipients, 6^{th} Edition; Editors R. C. Rowe, P. J. Sheskey and M. E. Quinn, The Pharmaceutical Press, London, American Pharmacists Association, Washington, 2009. Any suitable pharmaceutically acceptable excipient is within the scope of the invention.

Pharmaceutical compositions include those suitable for oral, nasal, topical (including buccal, sublingual and transdermal), parenteral (including subcutaneous, intravenous and intramuscular) or rectal administration. In some embodiments, the pharmaceutical composition is suitable for topical or oral administration, i.e. the pharmaceutical composition is a topical or oral formulation.

The pharmaceutical compositions may be compressed into solid dosage units, such as tablets, or be processed into capsules or suppositories. The pharmaceutical compositions may also be injected and may be prepared in the form of a solution, suspension or emulsion for such an application. Alternatively, the pharmaceutical compositions may be administered as a spray, including a nasal or buccal spray. Otherwise, the pharmaceutical compositions may be processed into a gel, cream, patch, implant or any other preparation for immediate and/or sustained release. Typically, the pharmaceutical compositions are processed into a gel, cream, lotion, foam or ointment for topical administration; or a tablet, capsule or buccal spray for oral administration.

The third aspect of the invention provides a compound of the first aspect or a pharmaceutical composition of the second aspect for use as a medicament. Specifically, the compounds are useful in the treatment of diseases or conditions associated with the activity of Bromodomain and Extra-Terminal proteins. In the fifth aspect, there is provided a compound of the first aspect or a pharmaceutical composition of the second aspect for use in the inhibition of Bromodomain and Extra-Terminal proteins. Diseases or conditions associated with the activity of Bromodomain and Extra-Terminal proteins include inflammatory skin disorders, respiratory diseases, gastrointestinal diseases, eye diseases, cancers, rheumatic diseases, demyelinating diseases and fibrotic diseases. Therefore, in the fourth aspect, the invention provides a compound of the invention or a pharmaceutical composition of the invention for use in a method of treatment or prophylaxis of inflammatory skin disorders, respiratory diseases, gastrointestinal diseases, eye diseases, cancers, rheumatic diseases, demyelinating diseases and fibrotic diseases and in the sixth aspect, the invention provides a method for the treatment or prophylaxis of skin disorders, respiratory diseases, gastrointestinal diseases, eye diseases, cancers, rheumatic diseases, demyelinating diseases and fibrotic diseases, said method comprising administering to a subject, an effective amount of a compound of the first aspect, or a pharmaceutical composition of the second aspect.

Inflammatory diseases rely on T helper cells Th₁, Th₂ and Th₁₇ for innate and adaptive immunity responses which affect either or both of the acute or chronic stages of the disease. Many cytokine and chemokines are upregulated in an inflammatory disease and the ability to reduce the levels of these inflammatory markers is evidence of the ability of a drug to ameliorate a disease. Such cytokine and chemokines include but are not limited to granulocyte-macrophage colony-stimulating factor (GM-CSF); interleukins IL-1, IL-2, IL-4, IL-6, IL-8, IL-13, IL-17, IL-22; chemokine (c-c motif) ligands CCL2, CCL27 and CCL20; tumour necrosis factor alpha (TNF-α); thymic stromal lymphopoietin (TSLP); and chemokine (c-x-c motif) ligand 9 (CXCL9).

Pan-BET inhibitors may be of value in the treatment of inflammatory disorders. These include skin disorders such as alopecia areata, Atopic dermatitis, bullous diseases, dermatitis, dermatitis herpetiformis, dermatomyositis, vitiligo, contact dermatitis, psoriasis, rosacea, scleroderma, xerosis, urticarial and chronic idiopathic pruritus and vitiligo; respiratory diseases such as asthma, chronic obstructive pulmonary disease, pulmonary fibrosis, cystic fibrosis, rhinitis, bronchiolitis, byssinosis, pneumoconiosis, bronchiectasis, hypersensitivity pneumonitis, mesothelioma, sarcoidosis; gastrointestinal diseases such as inflammatory bowel disease, ulcerative colitis, Crohn's disease, retroperitoneal fibrosis, celiac disease and gastrointestinal cancers; eye diseases such as myasthenia gravis, Sjögran's syndrome, conjunctivitis, scleritis, uveitis, dry eye syndrome, keratitis and iritis; systemic indications like Addison's disease, acute gout, ankylosing spondylitis, atherosclerosis, Behcet's disease, giant cell arthritis, glomerulonephritis, hepatitis, hypophysitis, lupus nephritis, Kawasaki disease, multiple sclerosis, myocarditis, myositis, nephritis, osteoarthritis, pancreatitis, pericarditis, polyarteritis nodosa, pneumonitis, primary biliary cirrhosis, psoriatic arthritis, rheumatoid arthritis, scleroderma (cutaneous or systemic), scleritis, sclerosing cholangitis, sepsis, systemic lupus erythematosus, Takayasu's arteritis, toxic shock, thyroiditis, type 1 diabetes and complications from diabetes, uvenitis, vasculitis and Wegener's granulomatosis; as well as other autoimmune diseases and indications where immunosuppression would be desirable for instance in organ transplantation.

BET inhibitors are also known to affect the growth or survival of a range of cancers, specifically skin and systemic cancers, and may be useful for the treatment of acoustic neuroma, acute leukaemia, acute lymphocytic leukaemia, acute myelocytic leukaemia (monocyctic, myeloblastic, adenocarcinoma, angiosarcoma, astrocytoma, myelomonocytic and promyelocytic), acute t-cell leukaemia, basal cell carcinoma, bile duct carcinoma, bladder cancer, brain cancer, breast cancer, bronchogenic carcinoma, cervical cancer, chondrosarcoma, chordoma, choriocarcinoma, chronic leukaemia, chronic lymphocytic leukemia, chronic myelocytic (granulocytic) leukaemia, chronic myelogenous leukaemia, colon cancer, colorectal cancer, craniopharyngioma, cystadenocarcinoma, cutaneous T-cell lymphoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukaemia, liposarcoma, lung cancer, lymphagioendotheliosarcoma, lymphangiosarcoma, lymphoblastic leukaemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukaemia, lymphoma, medullary carcinoma, medulloblastoma, melanoma, meningioma, mesothelioma, multiple myeloma, myelogenous leukaemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumours, uterine cancer, and Wilms' tumor.

BET inhibitors may also be of use in the treatment of obesity, dyslipidaemia, hypercholesterolemia, Alzheimer's disease, metabolic syndrome, hepatic steatosis, type II diabetes, insulin resistance, diabetic retinopathy or diabetic neuropathy.

The seventh aspect provides a method of inhibiting Bromodomain and Extra-Terminal protein activity in a subject, said method comprising administering to a subject an effective amount of a compound of the first aspect, or a pharmaceutical composition of the second aspect.

An effective amount of the compound may be administered to a subject topically, parenterally or enterally. The compound may be administered parenterally, sometimes by direct injection, which is typically intramuscular, subcutaneous or intraveneous. Typically, however, the compound is administered topically to the skin or mucuous membranes via a cream, gel, foam, lotion or ointment, or enterally via a tablet, capsule or buccal spray.

The subject may, and typically is, a human, and may be suffering from or liable to suffer from inflammatory skin disorders, respiratory diseases, gastrointestinal diseases and eye diseases. Treatment of said subject may comprise administering an effective amount of a compound of the invention. The term “effective amount” denotes an amount of the compound that ameliorates the above-noted diseases and thus produces the desired therapeutic or inhibitory effect.

The skilled person is aware that an effective amount is likely to vary with the particular compound of the invention, the subject and the administration procedure used. It is within the means and capacity of the skilled person to identify the effective amount of the compounds and compositions of the invention via routine work and experimentation.

Any discussion herein of documents, acts, materials, devices, articles or the like is not to be taken as an admission that any or all of these matters form part of the prior art base or were common general knowledge in the field relevant to the present disclosure as it existed before the priority date of each claim of this application.

It will be appreciated by those skilled in the art that numerous variations and/or modifications may be made to the invention as described herein without departing from the scope of the invention as described. The present embodiments are therefore to be considered for descriptive purposes and are not restrictive, and are not limited to the extent of that described in the embodiment. The person skilled in the art is to understand that the present embodiments may be read alone, or in combination, and may be combined with any one or a combination of the features described herein.

The subject-matter of each patent and non-patent literature reference cited herein is hereby incorporated by reference in its entirety.

The aspects and embodiments of this disclosure are further described in the following clauses:

1. A compound of formula (I):

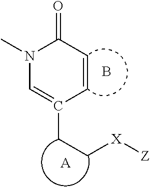

wherein ring structure A is a 5- or 6-membered aromatic or heteroaromatic ring, optionally substituted at one or more carbon and/or heteroatoms with a first substituent;
wherein each first substituent is independently selected from the group consisting of hydroxy, oxo, C₁-C₆alkyl, C₃-C₆cycloalkyl, C₁-C₆alkylol, halo, SO₂C₁-C₄alkyl, NHSO₂C₁-C₄alkyl, SO₂C₃-C₆cycloalkyl, NHSO₂C₃-C₆cycloalkyl, SO₂C₁-C₄alkylol, NHSO₂C₁-C₄alkylol, C₁-C₅alkyloxy, C₁-C₅alkylamino, SO₂NH₂, CONH₂, CONHC₁-C₄alkyl, NHCOC₁-C₄alkyl, NHSO₂N(C₁-C₄alkyl)₂, C₁-C₆fluoroalkyl, SO₂C₁-C₄fluoroalkyl, NHSO₂C₁-C₄fluoroalkyl, C₁-C₅fluoroalkyloxy, and C₁-C₅fluoroalkylamino;
X is O, CR², NR′ or S, wherein R is individually selected from the group consisting of H, C₁-C₄alkyl and halo, and R′ is selected from the group consisting of C₁-C₄alkyl and H;
Z is a 5- or 6-membered aromatic or heteroaromatic ring, C₁-C₆alkyl, C₃-C₆cycloalkyl, CR^{A}R^{B}R^{C}, C₂-C₅oxacycloalkyl, C₂-C₅azacycloalkyl or morpholinyl, optionally substituted at one or more carbon and/or heteroatoms with a second substituent;
wherein R^{A} is a C₃-C₅cycloalkyl, R^{B} is a C₃-C₅cycloalkyl, methyl or ethyl, and R^{C} is OH; and
each second substituent is independently selected from the group consisting of hydroxy, C₁-C₆alkyl, C₃-C₆cycloalkyl, halo, C₁-C₅alkyloxy, C₁-C₅alkylamino, oxo, cyano, C₁-C₆fluoroalkyl, C₁-C₅fluoroalkyloxy, and C₁-C₅fluoroalkylamino;
ring structure B is optionally present; wherein when ring structure B is present, it is an optionally substituted pyrrole bonded such that C is in the 4 position relative to NH; wherein the pyrrole is optionally substituted at position 2 with a third substituent;
wherein the third substituent is selected from the group consisting of CONHC₁-C₄alkyl, CONH₂, CONHC₁-C₆fluoroalkyl, CONHC₃-C₆cycloalkyl optionally substituted at one or more carbon atoms with a methyl or ethyl; CONHC₃-C₅cyclofluoroalkyl optionally substituted at one or more carbon atoms with a methyl or ethyl, NHCOC₁-C₄alkyl and NHCOC₁-C₄fluoroalkyl;
with the proviso that when A is 6-membered, it is substituted at least once with a hydroxy or oxo group.

2. The compound of clause 1, wherein the third substituent is selected from the group consisting of CONHC₁-C₄alkyl, CONH₂, CONHC₁-C₆fluoroalkyl, CONHC₃-C₅cycloalkyl; CONHC₃-C₅cyclofluoroalkyl, NHCOC₁-C₄alkyl and NHCOC₁-C₄fluoroalkyl.

3. The compound of clause 1 or clause 2, wherein when A is 6-membered, it is substituted at least once with a hydroxy group positioned ortho or meta to X, or an oxo group.

4. The compound of any one preceding clause, wherein the third substituent is CONHC₁-C₄alkyl.

5. The compound of any one preceding clause, wherein the third substituent is CONHethyl.

6. The compound of any one of clauses 1 to 3, wherein the compound is of formula (II):

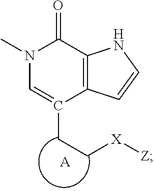

wherein A, X and Z are as defined for formula (I).

7. The compound of any preceding clause wherein A is selected from the group consisting of benzene, pyridine, thiazole, pyridone, pyrazole, imidazole and 1,2,4-triazole, optionally substituted at one or more carbon and/or heteroatoms with the first substituent.

8. The compound of clause 7 wherein the pyrazole carbon at position 5 is bound to C and the pyrazole nitrogen at position 1 is bound to X.

9. The compound of clause 7 or clause 8 wherein the imidazole carbon at position 2 is bound to C and the nitrogen at position 1 is bound to X.

10. The compound of any one of clauses 7 to 9 wherein the 1,2,4-triazole carbon at position 5 is bound to C and the nitrogen at position 1 is bound to X.

11. The compound of any one of clauses 1 to 6 wherein A is selected from the group consisting of benzene, pyridine, thiazole and pyridone, optionally substituted at one or more carbon and/or heteroatoms with the first substituent.

12. The compound of any one of clauses 7 to 11 wherein the pyridone is a 2-pyridone.

13. The compound of clause 12 wherein the 2-pyridone carbon at position 3 is bound to X and the 2-pyridone carbon at position 4 is bound to C; or the 2-pyridone carbon at position 5 is bound to X and the 2-pyridone carbon at position 4 is bound to C.

14. The compound of any one of clauses 7 to 13 wherein the thiazole carbon at position 4 is bound to C and the thiazole carbon at position 5 is bound to X.

15. The compound of any one preceding clause wherein each first substituent is independently selected from the group consisting of hydroxy, oxo, C₁-C₆alkyl, C₃-C₆cycloalkyl, C₁-C₆alkylol, halo, SO₂C₁-C₄alkyl, NHSO₂C₁-C₄alkyl, SO₂C₃-C₆cycloalkyl, NHSO₂C₃-C₆cycloalkyl, SO₂C₁-C₄alkylol, NHSO₂C₁-C₄alkylol, C₁-C₅alkyloxy and C₁-C₅alkylamino.

16. The compound of any one preceding clause wherein each first substituent is independently selected from the group consisting of hydroxy, oxo, C₁-C₆alkyl, C₃-C₆cycloalkyl, C₁-C₆alkylol and halo.

17. The compound of any one preceding clause wherein each first substituent is independently selected from the group consisting of hydroxy, oxo, methyl and halo.

18. The compound of any one preceding clause wherein R is individually selected from the group consisting of H, methyl and halo, and R′ is selected from the group consisting of methyl and H.

19. The compound of any one preceding clause wherein halo is fluoro or chloro.

20. The compound of any one preceding clause wherein R is individually selected from the group consisting of H, methyl and fluoro, and R′ is methyl.

21. The compound of any one preceding clause wherein X is O.

22. The compound of any one preceding clause wherein Z is a 5- or 6-membered aromatic or heteroaromatic ring, C₁-C₆alkyl, or C₃-C₆cycloalkyl, optionally substituted on one or more carbon or heteroatoms with a second substituent.

23. The compound of any one preceding clause wherein Z is a 6-membered aromatic or heteroaromatic ring, C₁-C₆alkyl, or C₃-C₆cycloalkyl, optionally substituted on one or more carbon or heteroatoms with a second substituent.

24. The compound of any one preceding clause, wherein Z is a 5- or 6-membered aromatic or heteroaromatic ring, optionally substituted on one or more carbon or heteroatoms with a second substituent

25. The compound of any one preceding clause, wherein Z is a 6-membered aromatic or heteroaromatic ring, optionally substituted on one or more carbon or heteroatoms with a second substituent

26. The compound of any one preceding clause wherein Z is a phenyl or pyridyl ring, optionally substituted at one or more carbon and/or nitrogen atoms with a second substituent.

27. The compound of any one preceding claim wherein Z is a phenyl ring, optionally substituted at one or more carbon and/or nitrogen atoms with a second substituent.

28. The compound of any one preceding clause wherein each second substituent is independently selected from the group consisting of hydroxy, C₁-C₆alkyl, C₃-C₆cycloalkyl, halo, C₁-C₅alkyloxy, C₁-C₅alkylamino, oxo and cyano.

29. The compound of any one preceding clause wherein each second substituent is independently selected from the group consisting of hydroxy, C₁-C₄alkyl and halo.

30. The compound of any one preceding clause wherein the second substituent is hydroxy.

31. The compound of clause 27 wherein each second substituent is independently selected from the group consisting of methyl and fluoro.

32. The compound of clause 27 wherein Z is a phenyl ring substituted with two methyl groups positioned ortho to X and one fluoro positioned para to X.

33. The compound of any one preceding clause, wherein C-A-X of formula (I) is any one of formulae (Ia), (Ib), (Ic), (Id) or (Id′):

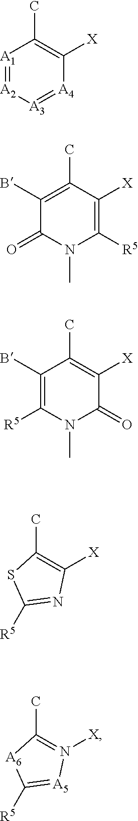

wherein A₁ is CR¹ or N, A₂ is CR² or N, A₃ is CR³ or N, A₄ is CR⁴, A₅ is CR⁵ or N and A₆ is CR⁵ or N;
R¹ is H or hydroxy;
R² is H, hydroxy, C₁-C₆alkyl, C₃-C₆cycloalkyl, halo, SO₂C₁-C₄alkyl, NHSO₂C₁-C₄alkyl, SO₂C₃-C₆cycloalkyl, NHSO₂C₃-C₆cycloalkyl, C₁-C₅alkyloxy or C₁-C₅alkylamino;
R³ and R⁴ are independently selected from the group consisting of H, hydroxy, C₁-C₆alkyl, C₃-C₅cycloalkyl, halo, C₁-C₅alkyloxy or C₁-C₅alkylamino;
with the proviso that at least one of R¹, R², R³ or R⁴ is hydroxy;
B′ is H or hydroxy; and
R⁵ is either H or the first substituent, defined above.

34. The compound of any one preceding clause wherein C-A-X is any one of formulae (Ia), (Ib), (Ic) or (Id):

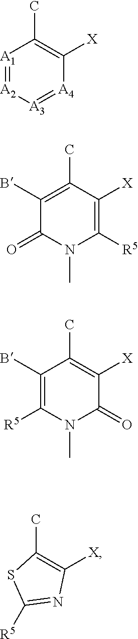

wherein A₁ is CR¹ or N, A₂ is CR² or N, A₃ is CR³ or N and A₄ is CR⁴;
R¹ is H or hydroxy;
R² is H, C₁-C₆alkyl, C₃-C₆cycloalkyl, halo, SO₂C₁-C₄alkyl, NHSO₂C₁-C₄alkyl, SO₂C₃-C₆cycloalkyl, NHSO₂C₃-C₆cycloalkyl, C₁-C₅alkyloxy or C₁-C₅alkylamino;
R³ and R⁴ are independently selected from the group consisting of H, hydroxy, C₁-C₆alkyl, C₃-C₅cycloalkyl, halo, C₁-C₅alkyloxy or C₁-C₅alkylamino;
with the proviso that at least one of R¹, R³ or R⁴ is hydroxy;
B′ is H or hydroxy; and
R⁵ is either H or the first substituent.

35. The compound of clause 33 or 34 wherein R² is H, C₁-C₅alkyl, halo, SO₂C₁-C₄alkyl or NHSO₂C₁-C₄alkyl; and R³ and R⁴ are independently selected from the group consisting of H, hydroxy, C₁-C₃alkyl and halo.

36. The compound of any one of clauses 33 to 35 wherein when CAX is represented by formula (Ia), Z is a phenyl ring, optionally substituted at one or more carbon atoms with a second substituent; and when CAX is represented by any one of formulae (Ib), (Ic), (Id) and (Id′), Z is a phenyl or pyridyl ring, optionally substituted at one or more carbon and/or nitrogen atoms with a second substituent.

37. The compound of any one of clauses 33 to 35 wherein when CAX is represented by formula (Ia), Z is an unsubstituted phenyl ring; and when CAX is represented by any one of formulae (Ib), (Ic), (Id) and (Id′), Z is a phenyl or pyridyl ring, optionally substituted at one or more carbon and/or nitrogen atoms with a second substituent selected from the group consisting of hydroxy, methyl, fluoro and chloro.

38 The compound of any one of clauses 33 to 37, wherein the compound is any one of:
(i) formula (Ia), (Id) or (Id′); or
(ii) formula (Ib) or (Ic).

39. The compound of any one of clauses 33 to 37, wherein the compound is any one of:
(i) formula (Ia) or (Id); or
(ii) formula (Ib) or (Ic).

40. The compound of clause 1, wherein the compound is any one of formulae (Ie) to (IIe):

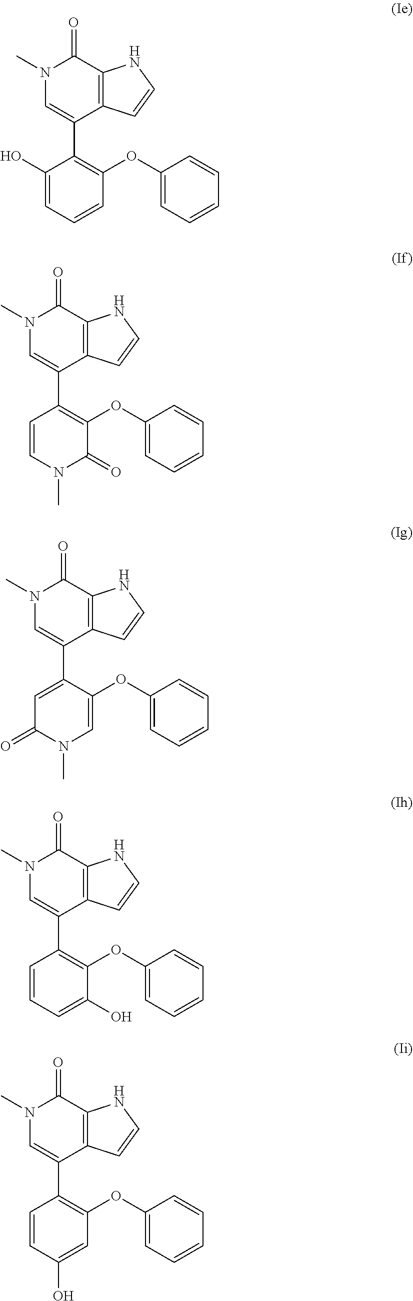

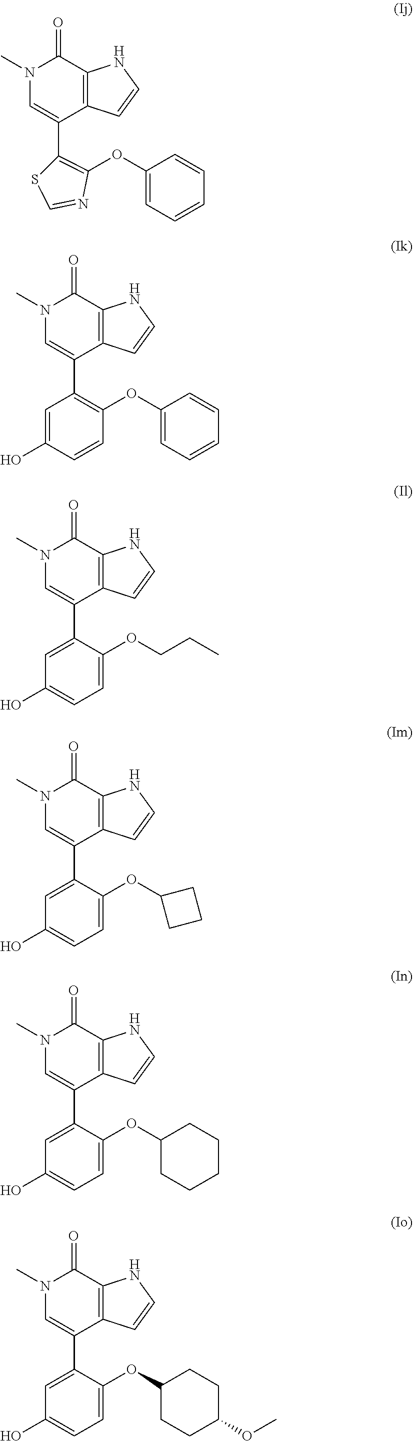

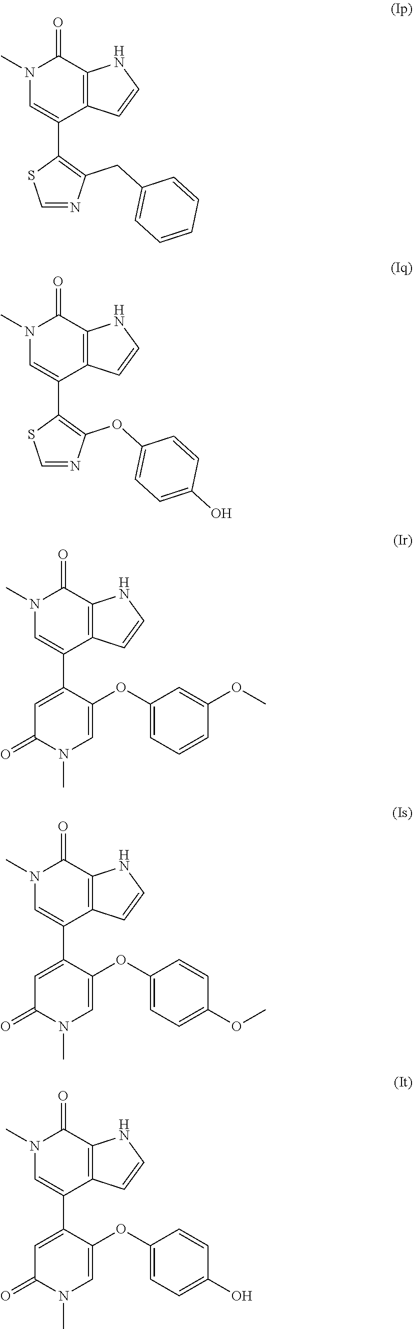

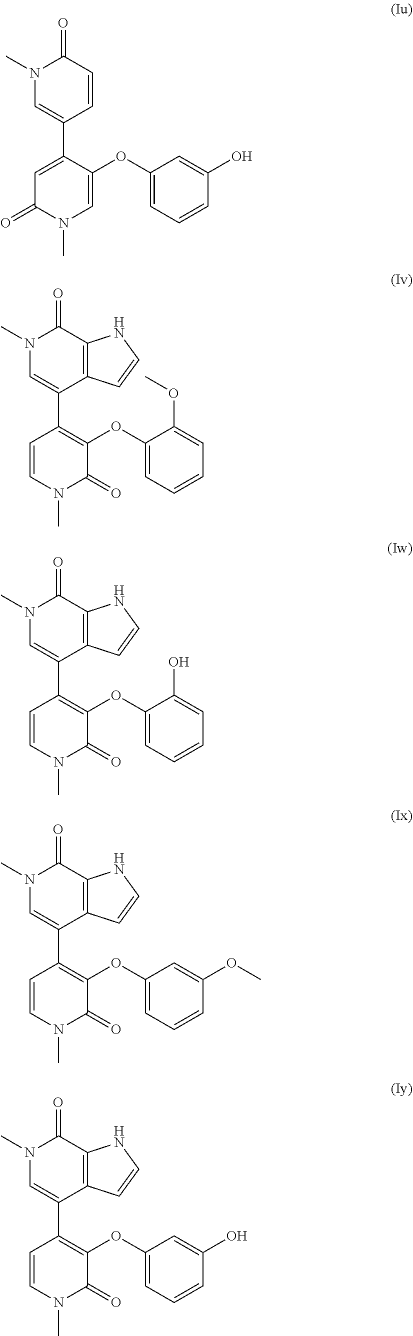

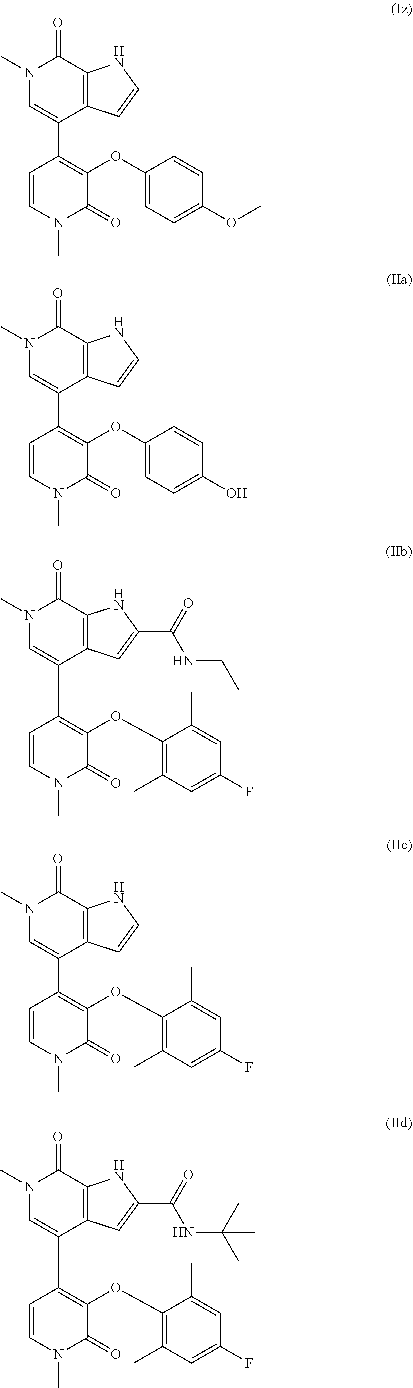

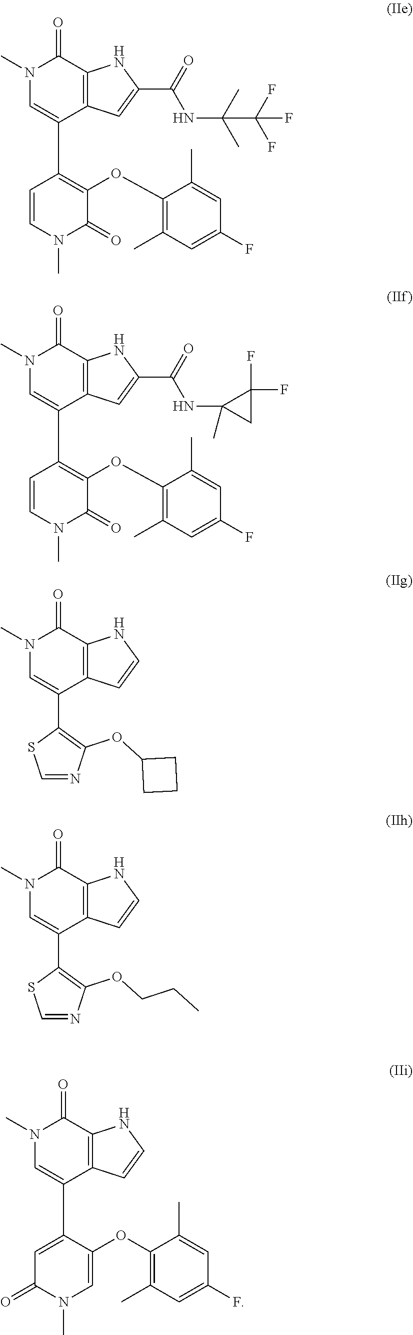

41. The compound of clause 40 wherein the compound is any one of formulae (Ie), (If), (Ig), (Ih), (Ii) (Ij) or (Ik).

42. The compound of any one preceding clause, in the form of a pharmaceutically acceptable salt.

43. The compound of formula (Ib) or (Ic), as defined in any one of clauses 33 to 41, in the form of a pharmaceutically acceptable salt.

44. The compound of formula (Ia), (Id) or (Id′), as defined in any one of clauses 33 to 41, in the form of a pharmaceutically acceptable salt.

44. The compound of formula (Ia) or (Id), as defined in any one of clauses 33 to 41, in the form of a pharmaceutically acceptable salt.

45. A pharmaceutical composition comprising any one or a combination of the compounds defined in any one of clauses 1 to 41, in combination with one or more pharmaceutically acceptable excipients.

46. The pharmaceutical composition of clause 45, wherein the pharmaceutical composition is a topical formulation.

47. A pharmaceutical composition comprising any one or a combination of the compounds of formula (Ia), (Id) or (Id′), as defined in any one of clauses 33 to 41, in combination with one or more pharmaceutically acceptable excipients.

48. A pharmaceutical composition comprising any one or a combination of the compounds of formula (Ia) or (Id), as defined in any one of clauses 33 to 41, in combination with one or more pharmaceutically acceptable excipients.

49. The pharmaceutical composition of clause 45, wherein the pharmaceutical composition is an oral formulation.

50. A pharmaceutical composition comprising any one or a combination of the compounds of formula (Ib) or (Ic), as defined in any one of clauses 33 to 41, in combination with one or more pharmaceutically acceptable excipients.

51. A compound as defined in any one of clauses 1 to 44, or a pharmaceutical composition as defined in any one of clauses 45 to 50, for use as a medicament.

52. A compound as defined in any one of clauses 1 to 44, or a pharmaceutical composition as defined in any one of clauses 45 to 50, for use in a method of treatment or prophylaxis of inflammatory skin disorders, respiratory diseases, gastrointestinal diseases, eye diseases, cancers, rheumatic diseases, demyelinating diseases and fibrotic diseases.

53. The compound or composition for the use of clause 52, wherein the use is in a method of treatment or prophylaxis of inflammation or cancer of the gut, skin or lung.

54. A compound as defined in any one of clauses 1 to 44, or a pharmaceutical composition as defined in any one of clauses 45 to 50, for use in the inhibition of Bromodomain and Extra-Terminal proteins.

55. A method for the treatment or prophylaxis of inflammatory skin disorders, respiratory diseases, gastrointestinal diseases, eye diseases, cancers, rheumatic diseases, demyelinating diseases and fibrotic diseases, said method comprising administering to a subject, an effective amount of a compound as defined in any one of clauses 1 to 44, or a pharmaceutical composition as defined in any one of clauses 45 to 50.

56. The method according to clause 55, wherein the method is for the treatment or prophylaxis of fibrosis of inflammation or cancer of the gut, skin or lung.

57. A method of inhibiting Bromodomain and Extra-Terminal protein activity in a subject, said method comprising administering to a subject an effective amount of a compound as defined in any one of clauses 1 to 44, or a pharmaceutical composition as defined any one of clauses 45 to 50.

The following are presented as non-limiting examples.

### EXAMPLES

It has been found that the compounds described herein are surprisingly effective as pan-inhibitors of BET BRDs. The examples disclosed herein exhibit nanomolar potency in inhibiting GM-CSF, IL-1a, IL-6, IL-8, CCL2, TNF-α, TSLP, CCL27, CCL20 and CXCL9 from stimulated keratinocytes. They also exhibit surprisingly effective clearance by human hepatocytes and solubility in formulations suitable for topical application. Advantageously for topical administration, the exemplified compounds are stable in human skin S9 fractions and under hydrolytic conditions at a range of pHs. Furthermore, exemplified topical formulations deliver practicable concentrations of the compound into the epidermis of the skin and the exemplified compounds are not toxic to primary keratinocytes.

Abbreviations

| | |
|---|---|
| APCI | atmospheric pressure chemical ionisation mass spectrum |
| BD | binding domain |
| br | broad |
| c | centi |
| CCL | chemokine (C-C motif) ligand |
| CXCL | chemokine (C-X-C motif) ligand |
| δ | chemical shift |
| d | doublet |
| dd | double doublet |
| DCM | dichloromethane |
| DMF | dimethylformamide |
| DMA | dimethylacetamide |
| DMSO | dimethyl sulfoxide |
| EC | effective concentration |
| ES | electrospray |
| ESI | electrospray ionization |
| g | gram |
| GM-CSF | granulocyte-macrophage colony-stimulating factor |
| hr | hour |
| HPLC | high performance liquid chromatography |
| HRMS | high resolution mass spectrum |
| IL | interleukin |
| J | coupling constant |
| K_{d} | dissociation constant |
| L | litre |
| LC | liquid chromatography |
| LG | leaving group |
| m | multiplet |
| m | milli |
| m | meter |
| M | molar |
| M+ | molecular ion |
| MHz | megahertz |
| min | minutes |
| mol | mole |
| MS | mass spectrometry |
| m/z | mass/charge |
| n | nano |
| NMR | nuclear magnetic resonance |
| p | para |
| PTSA | p-Toluenesulfonic acid |
| q | quartet |
| Rf | retardation factor |
| rpm | revolutions per minute |
| RT | room temperature |
| s | singlet |
| SM | starting material |
| S_{N}Ar | nucleophilic aromatic substitution (addition-elimination) |
| t | triplet |
| THF | tetrahydrofuran |
| TLC | thin layer chromatography |
| TLR | toll-like receptors |
| TBME | methyl tert-butyl ether |
| t_{R} | retention time |
| TSLP | thymic stromal lymphopoietin |
| TNF | tumor necrosis factor |
| XPhos | 2-dicyclohexylphosphino-2′,4′,6′-triisopropylbiphenyl |
| Ts or Tosyl | toluenesulfonyl |
| DTT | dithioreitol |
| BSA | bovine serum albumin |
| PBS | phosphate-buffered saline |
| MEG | monoethylene glycol |
| NADPH | nicotinamide adenine dinucleotide phosphate |
| μ | micro |
| UDPGA | uridine diphosphate glucuronic acid |
| UPLC | ultra performance liquid chromatography |
| UV | ultraviolet |
| vis | visable |
| w/w | weight by weight |
| ° C. | degree Celsius |
| % | per cent |

### Equipment

Reactions using microwave irradiation were carried out in a Biotage Initiator microwave.

Normal phase TLCs were carried out on pre-coated silica plates (Kieselgel 60 F₂₅₄, BDH) with visualisation via U.V. light (UV254/365 nm) and/or ninhydrin solution.

Flash chromatography was performed using Combiflash Companion Rf (Teledyne ISCO) and prepacked silica gel columns purchased from Grace Davison Discovery Science or SiliCycle.

Mass-directed preparative HPLC separations were performed using a Waters HPLC (2545 binary gradient pumps, 515 HPLC make up pump, 2767 sample manager) connected to a Waters 2998 photodiode array and a Waters 3100 mass detector.

Preparative HPLC separations were performed with a Gilson HPLC (321 pumps, 819 injection module, 215 liquid handler/injector) connected to a Gilson 155 UV/vis detector. On both instruments, HPLC chromatographic separations were conducted using Waters XBridge C18 columns, 19×100 mm, 5 μm particle size; using 0.1% ammonia in water (solvent A) and acetonitrile (solvent B) as mobile phase.

¹H NMR and ¹⁹F NMR spectra were recorded on a Bruker Avance DPX 500 spectrometer (¹H at 500.1 MHz, ¹³C at 125 MHz ¹⁹F at 470.5 MHz), or a Bruker Avance DPX 300 (¹H at 300 MHz). Chemical shifts (0) are expressed in ppm recorded using the residual solvent as the internal reference in all cases. Signal splitting patterns are described as singlet(s), doublet (d), triplet (t), quartet (q), multiplet (m), broad (br), or a combination thereof. Coupling constants (J) are quoted to the nearest 0.5 Hz. Low resolution electrospray (ES) mass spectra were recorded on a Bruker MicroTof mass spectrometer, run in positive mode. High resolution mass spectroscopy (HRMS) was performed using a Bruker MicroTof mass spectrometer.

LC-MS analysis and chromatographic separation were conducted with an Agilent Technologies 1200 series HPLC connected to an Agilent Technologies 6130 quadrupole LC/MS, connected to an Agilent diode array detector. The column used was a Waters XBridge column (50 mm×2.1 mm, 3.5 μm particle size,) and the compounds were eluted with a gradient of 5 to 95% acetonitrile/water+0.1% formic acid or a Shimadzu HPLC connected to a LCMS-2020 quadrupole LC/MS, connected to a Shimadzu diode array detector. The column used was a Kinetex EVO C18 column (30 mm×1.8 mm, 5.0 μm particle size) and the compounds were eluted with a gradient of 5 to 95% acetonitrile/water+0.0375% trifluoroacetic acid.

Unless otherwise stated herein, reactions have not been optimised. Solvents and reagents were purchased from commercial suppliers and used without further purification. Dry solvents were purchased in sure sealed bottles stored over molecular sieves.

Preparations and compounds have been named using the ChemDraw Professional 15.0 naming application.

### Process for Preparation

The following schemes illustrate methods of synthesising the compounds of the invention. Scheme 1 illustrates a general route for the preparation of compounds of the invention via Suzuki coupling of intermediates (II) and (VIII) followed by deprotection. The 6-methyl-1,6-dihydro-7H-pyrrolo[2,3-c]pyridin-7-one boronic ester intermediate (II) is prepared as follows:

5-bromo-2-methoxy-4-methyl-3-nitropyridine is reacted with DMF-DMA to give intermediate (VII). An iron catalysed reduction of the 3-nitro group to the corresponding amine initiates ring closure to give intermediate (VI). Tosyl protection followed by acid hydrolysis with HBr gives intermediate (IV). The pyridone group is then N-methylated with methyl iodide and sodium hydride to give intermediate (III). Intermediate (II) is then formed from the 4-bromoaryl compound (III) via treatment with 4,4,4′,4′,5,5,5′,5′-Octamethyl-2,2′-bi-1,3,2-dioxaborolane in a palladium-catalysed coupling reaction. Suzuki coupling of (II) and (VIII), followed by deprotection, produces compound (I). Deprotection involves removal of the tosyl group of intermediate (II) using, for example, sodium hydroxide. Often, deprotection also involves conversion of a methoxy substituent on A and/or Z to a hydroxy group using, for example, boron tribromide.

Alternatively, the compound may be functionalised at position 2 of the pyrrole with a third substituent, typically CONHethyl. This may be carried out by using an alternative synthetic pathway, shown in Scheme 2, in which intermediate (III) is reacted with ethylchloroformate and a strong base, such as lithium diisopropylamide (LDA) to form intermediate (III′). Intermediate (II′) is then formed from compound (III′) via treatment with 4,4,4′,4′,5,5,5′,5′-Octamethyl-2,2′-bi-1,3,2-dioxaborolane in a palladium-catalysed coupling reaction. Suzuki coupling of (II′) and (VIII), followed by deprotection, produces compound (I′). Deprotection involves removal of the tosyl group using, for example, sodium hydroxide. This also converts the ethoxy substituent of the ethyl formyl to a hydroxy group. Finally, the carboxylic acid at position 2 of the pyrrole of intermediate (I′) is reacted with a suitable amine to produce the desired third substituent. Oxalyl chloride is typically used to catalyse this reaction step by first converting the carboxylic acid to an acyl chloride, which is more susceptible to nucleophilic substitution with an amine. The skilled person is able to assess which amines and reaction conditions are suitable to functionalise the carboxylic acid of intermediate (I′) to produce compound (I″).

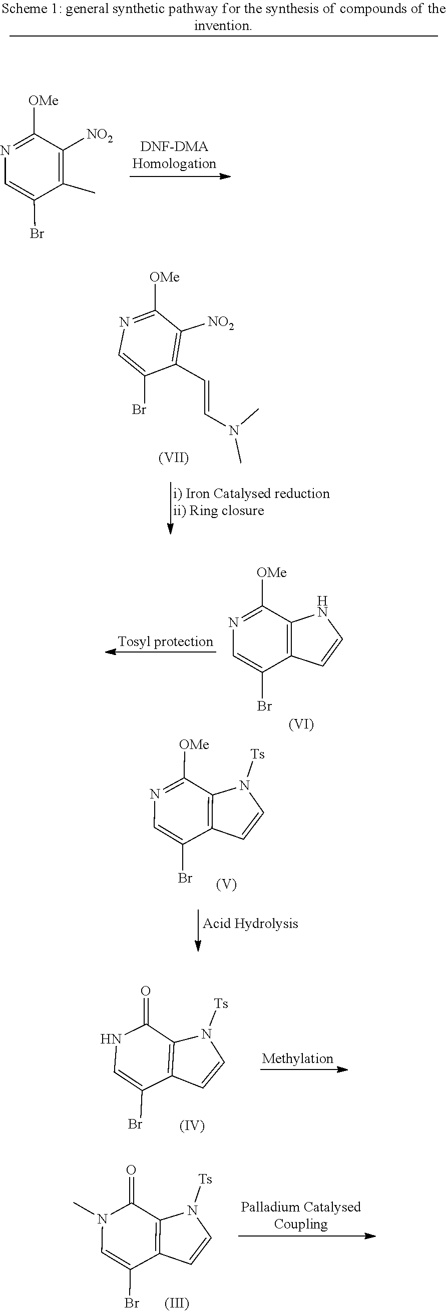

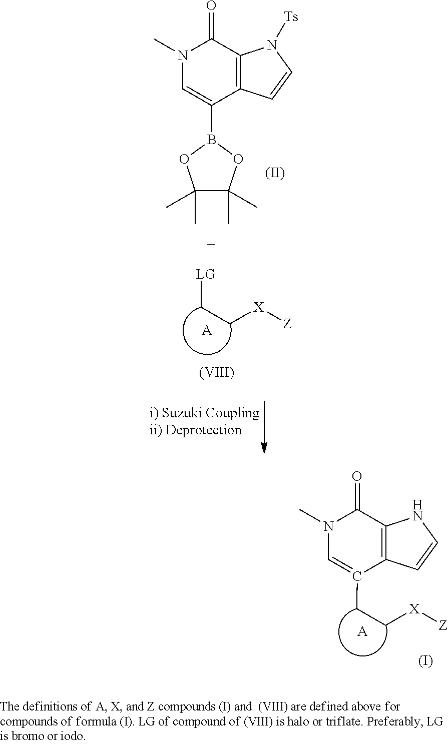

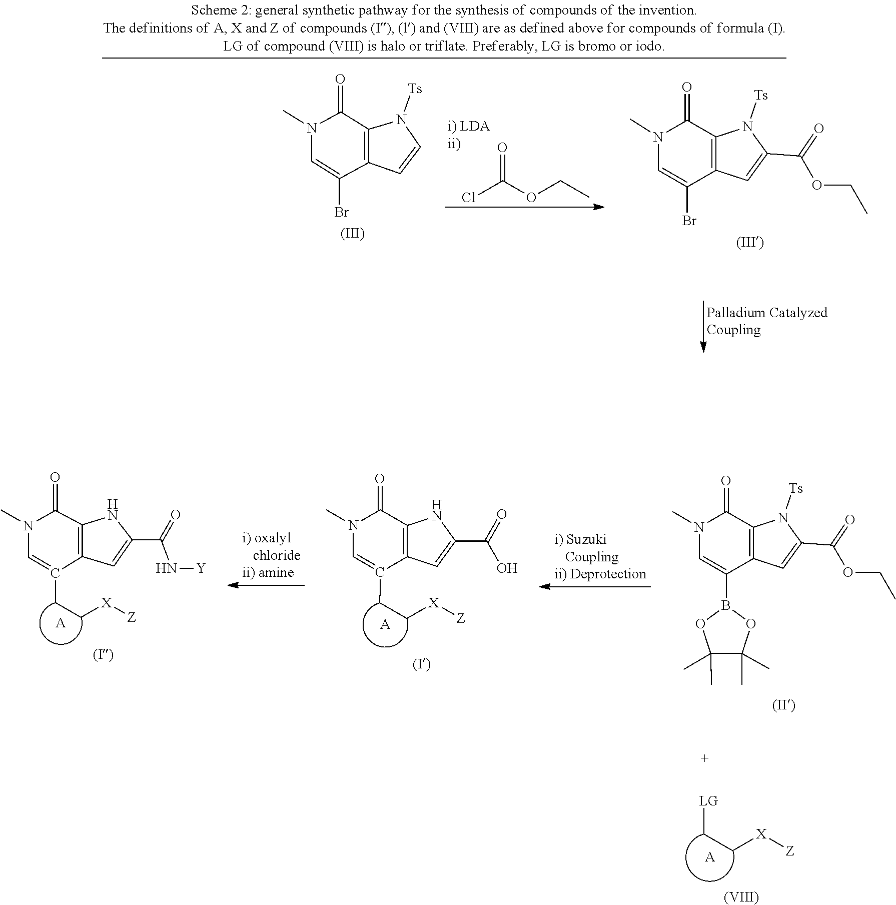

The use of Scheme 2 to synthesise compounds described herein wherein A is 2-pyridone, X is oxide and Z is 2,6-dimethyl-4-fluoro-phenyl, such as Example 41, is shown in Scheme 3.

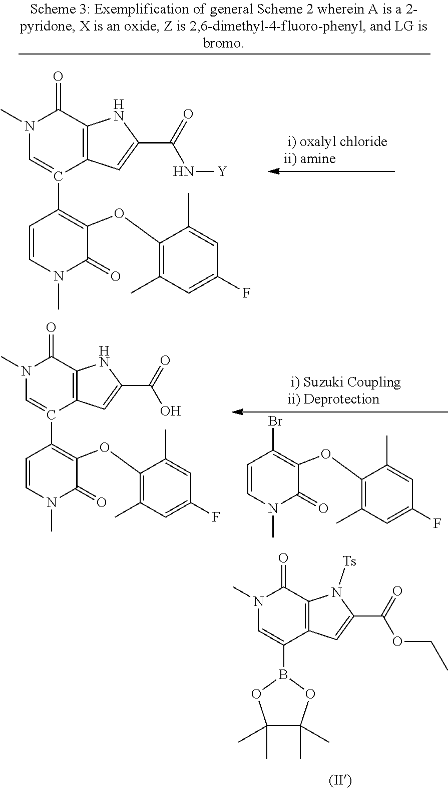

The synthetic pathways suitable for the synthesis of intermediates of formula (VIII) of Schemes 1 and 2 depend on the identity of A. Suitable pathways to synthesise intermediates of formula (VIII) are shown in Schemes 4, 5 and 6, in which: A is a 6-membered aromatic or heteroaromatic ring optionally substituted at one or more carbon and/or heteroatoms with the first substituent and substituted at a minimum of one carbon atom with a hydroxy or oxo group; A is an N-methyl-2-pyridone optionally substituted with a hydroxy group; and A is a thiazole.

Iodo-intermediate (IX) is prepared via a Sandmeyer reaction of the corresponding aniline (X), which is in turn formed through an iron catalysed reduction of the nitro-containing compound (XI). (XI) is formed via a SNAr reaction of the ortho-fluoro nitroaryl compound (XII).

N-methyl-2-pyrone-intermediates (XIII) are prepared via methylation and iron-catalysed oxidation of the corresponding pyridines (XIV), which are in turn formed via reduction of the corresponding pyridine oxides (XV) using phosphorus tribromide. Bromo-intermediates (XV) are prepared via reaction of the corresponding nitro intermediates (XVI) with acetyl bromide, and compounds (XVI) are in turn produced via SNAr reaction of the corresponding ortho-fluoro nitro pyridine oxide compounds (XVII).

5-bromothiazole intermediate (XVIII) is prepared via bromination of the corresponding thiazole intermediate (XIX), which is in turn prepared via the copper-catalysed Ullmann-type reaction of the corresponding bromo compound (XX).

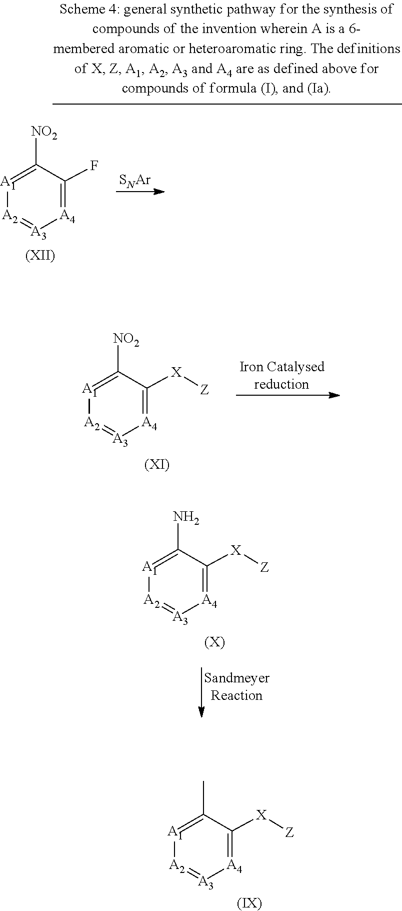

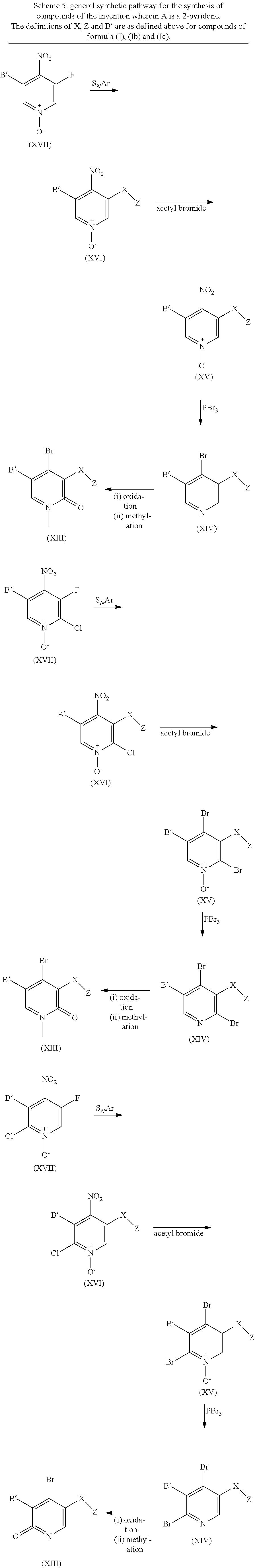

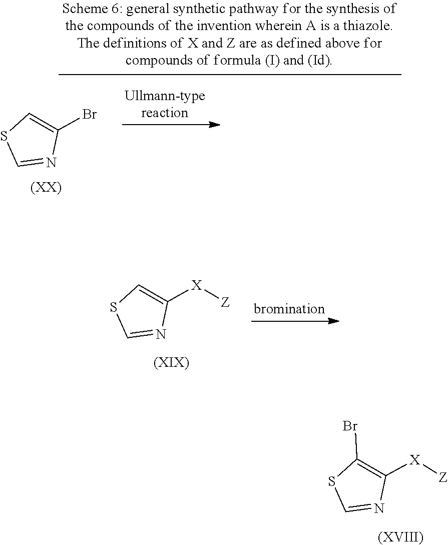

Typically, X is O (an oxide). Scheme 7 illustrates suitable reagents and reaction conditions for the preparation of the compound of Example 1. The skilled person is aware that the reagents and conditions employed in the chemical transformations of Scheme 7 may be utilised, modified and/or substituted for alternatives as necessary in order to furnish various alternative compounds via the general processes in Schemes 1 and 2.

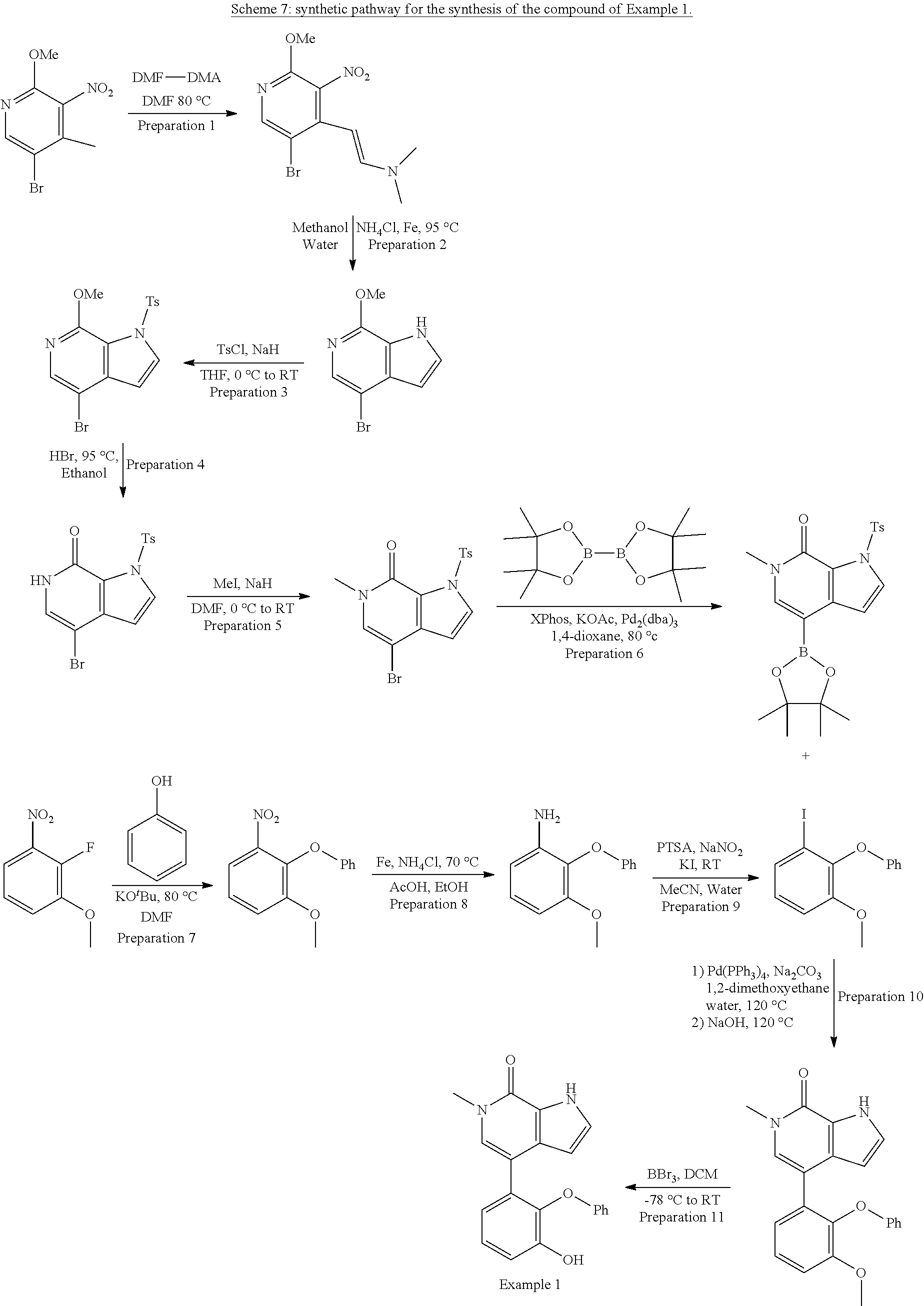

### Example 1: 4-(3-hydroxy-2-phenoxyphenyl)-6-methyl-1,6-dihydro-7H-pyrrolo[2,3-c]pyridin-7-one

### Preparation 1: (E)-2-(5-bromo-2-methoxy-3-nitropyridin-4-yl)-N,N-dimethylethene-1-amine

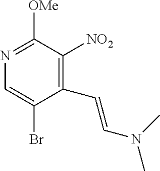 [SMILES: COc1ncc(Br)c(/C=C/N(C)C)c1[N+](=O)[O-]]

5-bromo-2-methoxy-4-methyl-3-nitropyridine (50 g, 202 mmol) was dissolved in DMF (410 mL) under nitrogen and heated to 80 °C. DMF-DMA (224 mL, 1.686 mol) was added over a period of 20 min. The resulting dark solution was heated at 95 °C. TLC (4:1 heptane/EA) after 5 hr showed no SM remaining. The mixture was cooled to RT and poured into ice water (1100 mL). The resulting suspension was stirred for 15 min then filtered. The collected red solid was washed with water and dried overnight under vacuum at 50 °C (56.6 g, 61%). The material was used directly in preparation 2 without further purification.

¹H NMR (400 MHz, CDCl₃) δ 8.14 (s, 1H), 7.02 (d, J=13.7 Hz, 1H), 4.94 (d, J=13.7 Hz, 1H), 3.97 (s, 3H), 2.94 (s, 6H).

### Preparation 2: 4-bromo-7-methoxy-1H-pyrrolo[2,3-c]pyridine

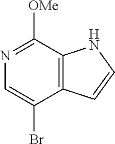 [SMILES: COc1ncc(Br)c2cc[nH]c12]

(E)-2-(5-bromo-2-methoxy-3-nitropyridin-4-yl)-N,N-dimethylethene-1-amine (23.3 g, 77.1 mmol) was partially dissolved in methanol (1100 mL) and ammonium chloride (23.3 g, 436 mmol), followed by water (140 mL). Iron powder (23.3 g, 417 mmol) was added and the mixture heated at reflux. The reaction mixture was stirred using an overhead stirrer. After 5 hr a further aliquot of iron powder (23.3 g, 417 mmol) was added and heating continued overnight. The mixture was cooled and solid Na₂CO₃ was added. The mixture was filtered through a pad of celite. The filtrate was filtered and the residue triturated with 4:1 heptane/Ethyl acetate. The mixture was filtered through a pad of silica. The filtrate was evaporated. The residue was purified on silica, eluting with 100:0 to 80:20 heptane/ethyl acetate. Solvent reduction gave an off-white solid (3.7 g, 21%).

HPLC t_{R} (Agilent, acidic, 3.5 min): 1.46 min, MS: m/z 229.0 [M+2H]⁺.

### Preparation 3: 4-bromo-7-methoxy-1-tosyl-1H-pyrrolo[2,3-c]pyridine

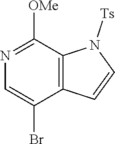 [SMILES: COc1ncc(Br)c2ccn(S(=O)(=O)c3ccc(C)cc3)c12]

Sodium hydride (60% w/w, 7.90 g, 198 mmol) was suspended in THF (290 mL) under nitrogen and was cooled to below 4 °C in an ice bath. 4-bromo-7-methoxy-1H-pyrrolo[2,3-c]pyridine (14.0 g, 61.7 mmol) was dissolved in THF (290 mL) and added dropwise over a period of 30 min (evolution of gas was observed and formation of an exotherm raised the reaction temperature to 5 °C). The maroon mixture was stirred at RT for 45 min before cooling to 3 °C. 4-Methylbenzenesulfonyl chloride (15.7 g, 82.1 mmol) was dissolved in THF (290 mL) and added dropwise. The resulting grey suspension was stirred 1.5 hr with cooling, and then 1 hr at RT. TLC (3:2 heptane/ethyl acetate) showed no remaining SM. The reaction mixture was quenched by dropwise addition of sat NH₄Cl (300 mL). The mixture was stirred 5 min before separating the phases. The aqueous phase was extracted with ethyl acetate (2×300 mL). The combined organics were washed (brine), dried (MgSO₄), filtered and evaporated to an oil that crystallized on cooling to give a light tan solid (26.2 g 99%). The material was used directly in preparation 4 without further purification.

HPLC t_{R} (Agilent, acidic, 3.5 min): 1.94 min, m/z=383.1 [M+2H]⁺.

### Preparation 4: 4-bromo-1-tosyl-1,6-dihydro-7H-pyrrolo[2,3-c]pyridin-7-one

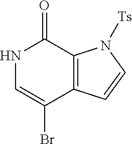 [SMILES: Cc1ccc(S(=O)(=O)n2ccc3c(Br)c[nH]c(=O)c32)cc1]

4-bromo-7-methoxy-1-tosyl-1H-pyrrolo[2,3-c]pyridine (26.2 g, 65.3 mmol) was suspended in ethanol (50 mL) and hydrogen bromide (48% w/w, 280 mL) was added in a steady stream. The resulting mixture was heated at 90 °C. TLC (3:2 heptane/ethyl acetate) after 2 hr showed no remaining SM. The reaction mixture was cooled to RT and then cooled in an ice bath with stirring for 30 min. The mixture was filtered and the cream coloured solid was collected and washed with water. The solid was dried overnight under vacuum at 50 °C (22.5 g, 94%). The material was used directly in preparation 5 without further purification.

HPLC t_{R} (Agilent, acidic, 3.5 min): 1.59 min, m/z=369.0 [M+2H]⁺.

### Preparation 5: 4-bromo-6-methyl-1-tosyl-1,6-dihydro-7H-pyrrolo[2,3-c]pyridin-7-one

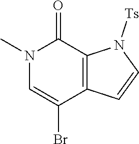 [SMILES: Cc1ccc(S(=O)(=O)n2ccc3c(Br)cn(C)c(=O)c32)cc1]

4-bromo-1-tosyl-1,6-dihydro-7H-pyrrolo[2,3-c]pyridin-7-one (22.5 g, 61.3 mmol) was dissolved in DMF (225 mL) under nitrogen. The mixture was cooled to 3 °C and sodium hydride (60% w/w, 3.06 g, 76.6 mmol) added in small portions, producing an evolution of gas and exotherm to 5 °C. The mixture was stirred for 20 min with cooling where after the evolution of gas had ceased, iodomethane (7.63 mL, 123 mmol) was added dropwise, producing an exotherm which raised the reaction temperature to 10 °C. The mixture was stirred for 15 min with cooling, then for 15 min at RT. LCMS after 2 hr showed no SM remaining. The reaction mixture was quenched by dropwise addition of water (100 mL, evolution of gas and exotherm to 39 °C). The mixture was extracted with ethyl acetate (3×300 mL). The combined organics were washed (brine), dried (Na₂SO₄), filtered and evaporated. The crude product was triturated with TBME and filtered. The collected off-white solid was washed with TBME and dried under vacuum (15 g, 64%).

HPLC t_{R} (Agilent, basic, 6.0 min): 4.0 min, m/z=382.9 [M+H]⁺.

### Preparation 6: 6-methyl-4-(4,4,5,5-tetramethyl-1,3,2-dioxaborolan-2-yl)-1-tosyl-1,6-dihydro-7H-pyrrolo[2,3-c]pyridin-7-one

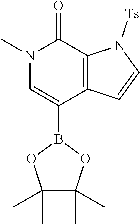 [SMILES: Cc1ccc(S(=O)(=O)n2ccc3c(B4OC(C)(C)C(C)(C)O4)cn(C)c(=O)c32)cc1]

To a flask containing XPhos (625.22 mg, 1.31 mmol), 4-bromo-6-methyl-1-tosyl-1,6-dihydro-7H-pyrrolo[2,3-c]pyridin-7-one (5 g, 13.1 mmol), 4,4,5,5-tetramethyl-2-(4,4,5,5-tetramethyl-1,3,2-dioxaborolan-2-yl)-1,3,2-dioxaborolane (6.66 g, 26.23 mmol) and potassium acetate (2.83 g, 28.85 mmol) was added 1,4-Dioxane (100 mL) and the suspension was degassed for 10 min. Pd₂(dba)₃ (300 mg, 0.32 mmol) was added and the mixture degassed for 1 min more. The reaction was heated at 80 °C overnight. The reaction was diluted with ethyl acetate and washed with 50% brine. The organics were dried, filtered and concentrated to a yellow/brown oil. The product was purified by flash chromatography on silica gel (80 g) eluting with ethyl acetate/heptane gradient (0-80%). Fractions corresponding to product were combined and concentrated to give a yellow solid (3.4 g, 55%).

HPLC t_{R} (Agilent, acidic, 3.5 min): 1.93 min, m/z=429.2 [M+H]⁺.

### Preparation 7: 1-methoxy-3-nitro-2-phenoxybenzene

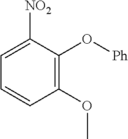 [SMILES: COc1cccc([N+](=O)[O-])c1Oc1ccccc1]

Phenol (2.3 g, 24.10 mmol) and potassium tert-butoxide (2.7 g, 24.10 mmol) were dissolved in DMF (40 mL) and stirred for 30 min at RT before the addition of 2-fluoro-1-methoxy-3-nitro-benzene (3.75 g, 21.91 mmol). The reaction mixture was heated to 80 °C and allowed to stir overnight. The reaction mixture was concentrated under vacuum, then re-dissolved in ethyl acetate and washed with water. The organic phase was dried over MgSO₄ and evaporated in vacuo. The crude material was purified by column chromatography (0-100% ethyl acetate in heptane) to afford 1-methoxy-3-nitro-2-phenoxy-benzene (5.4 g, 90%) as a yellow solid.

¹H NMR (500 MHz, CDCl₃) δ 7.55 (dd, J=1.4, 8.3 Hz, 1H), 7.35-7.30 (m, 3H), 7.24 (dd, J=1.4, 8.4 Hz, 1H), 7.11-7.05 (m, 1H), 6.89-6.87 (m, 2H), 3.93 (s, 3H).

### Preparation 8: 3-methoxy-2-phenoxyaniline

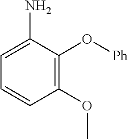 [SMILES: COc1cccc(N)c1Oc1ccccc1]

A solution of iron (7.37 g, 132.12 mmol) in acetic acid (12 mL)/ethanol (30 mL), was degassed with nitrogen for 5 min. 1-methoxy-3-nitro-2-phenoxy-benzene (5.4 g, 22.02 mmol) was added and the reaction heated to 70 °C. The orange solution went black after 5 min. After 4 hrs the reaction was allowed to cool and the solvent removed. DCM was added and the organic layer was washed with sodium bicarbonate, passed through a hydrophobic frit and concentrated. The product was purified by column chromatography (ethyl acetate/heptane gradient 0-100%). Fractions corresponding to product were combined and concentrated to provide 3-methoxy-2-phenoxyaniline (830 mg, 15%) as a brown solid.

HPLC t_{R} (Agilent, acidic, 3.5 min): 1.53 min, m/z=216.2 [M+H]⁺.

### Preparation 9: 1-iodo-3-methoxy-2-phenoxybenzene

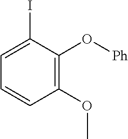 [SMILES: COc1cccc(I)c1Oc1ccccc1]

To a solution of 3-methoxy-2-phenoxyaniline (770 mg, 3.57 mmol) in MeCN (21 mL), and water (12 mL), p-toluenesulfonic acid monohydrate (2.0 g, 10.72 mmol) was added and the reaction mixture was stirred vigorously. A solution of potassium iodide (1.48 g, 8.93 mmol) and sodium nitrite (0.49 g, 7.13 mmol) in water (12 mL) was added dropwise over 10 min. The solution turned brown, and was stirred for 1 hr. Saturated sodium bicarbonate solution was then added to the solution until a pH of 8 was achieved. 1M sodium thiosulphate solution was then added. The product was extracted into DCM and the organics collected via phase separator and concentrated. The product was purified by flash chromatography on silica gel (12 g) eluting with an ethyl acetate/heptane gradient (0-40%). Fractions corresponding to product were combined and concentrated to provide 1-iodo-3-methoxy-2-phenoxybenzene (740 mg, 57%).

¹H NMR (500 MHz, CDCl₃) δ 7.43 (dd, J=1.4, 8.3 Hz, 1H), 7.34-7.28 (m, 3H), 7.10-7.04 (m, 1H), 6.99 (dd, J=1.4, 8.4 Hz, 1H), 6.88-6.85 (m, 2H), 3.93 (s, 3H).

### Preparation 10: 4-(3-methoxy-2-phenoxyphenyl)-6-methyl-1,6-dihydro-7H-pyrrolo[2,3-c]pyridin-7-one

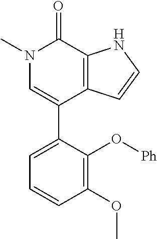 [SMILES: COc1cccc(-c2cn(C)c(=O)c3[nH]ccc23)c1Oc1ccccc1]

In a microwave tube, 1-iodo-3-methoxy-2-phenoxybenzene (83.7 mg, 0.25 mmol), sodium carbonate (81.6 mg, 0.77 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (110 mg, 0.25 mmol) in 1,2-Dimethoxyethane (2 mL) and Water (1 mL) was degassed by bubbling nitrogen for 10 min. Pd(PPh₃)₄ (14.83 mg, 0.013 mmol) was added, the tube sealed and the reaction heated at 120 °C for 30 min. NaOH (53 mg, 1.25 mmol) was added and the reaction heated at 120 °C for 1 h. Ethyl acetate (50 ml) was added and the organics washed with 2×50 ml water then 1×50 ml saturated brine solution. The organics were then separated and dried (MgSO₄) before concentration to dryness. The crude was then purified by flash column chromatography eluting with ethyl acetate/heptane gradient (0-100%). The desired fractions were combined and dried to afford 4-(3-methoxy-2-phenoxyphenyl)-6-methyl-1,6-dihydro-7H-pyrrolo[2,3-c]pyridin-7-one (22 mg, 26%) as a white solid. HPLC t_{R} (Agilent, acidic, 3.5 min): 1.62 min, m/z=347.5 [M+H]⁺.

### Preparation 11: 4-(3-hydroxy-2-phenoxyphenyl)-6-methyl-1,6-dihydro-7H-pyrrolo[2,3-c]pyridin-7-one

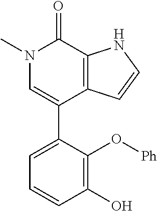 [SMILES: Cn1cc(-c2cccc(O)c2Oc2ccccc2)c2cc[nH]c2c1=O]

4-(3-methoxy-2-phenoxyphenyl)-6-methyl-1,6-dihydro-7H-pyrrolo[2,3-c]pyridin-7-one (21 mg, 0.06 mmol) was dissolved in DCM (2 mL) and cooled to −78 °C before the addition of BBr₃ (74 mg, 0.29 mmol). The reaction temperature was maintained for 1 hr and then allowed to warm to 0 °C and stirred for a further 1 hr. The reaction mixture was quenched with water and the pH was adjusted to 8 with saturated aqueous NaHCO₃. The reaction mixture was extracted with ethyl acetate. The combined organic layers were separated, passed through a phase separator and concentrated under vacuum. The crude material was purified by column chromatography (0-50% 20% MeOH/DCM in DCM) followed by reverse phase preparative HPLC (Gilson acidic 60-90% gradient). Fractions were concentrated on the genevac overnight to afford 4-(3-hydroxy-2-phenoxyphenyl)-6-methyl-1,6-dihydro-7H-pyrrolo[2,3-c]pyridin-7-one (17 mg, 85%) as a white solid.

HPLC t_{R} (Agilent, acidic, 3.5 min): 1.43 min, m/z=333.2 [M+H]⁺.

¹H NMR (500 MHz, CDCl₃) δ 10.36-10.30 (m, 1H), 7.26-7.20 (m, 2H), 7.15-7.06 (m, 4H), 6.89-6.85 (m, 2H), 6.68-6.64 (m, 2H), 6.39 (dd, J=2.4, 2.4 Hz, 1H), 6.29 (s, 1H), 3.53 (s, 3H).

### Example 2: 4-(4-hydroxy-2-phenoxyphenyl)-6-methyl-1,6-dihydro-7H-pyrrolo[2,3-c]pyridin-7-one

### Preparation 12: 4-methoxy-1-nitro-2-phenoxybenzene

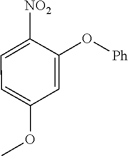 [SMILES: COc1ccc([N+](=O)[O-])c(Oc2ccccc2)c1]

Following the procedure in preparation 7, 2-fluoro-4-methoxy-1-nitrobenzene (400 mg, 2.34 mmol) was reacted to give the title compound (541 mg, 85%).

¹H NMR (500 MHz, CDCl₃) δ 8.10 (d, J=9.2 Hz, 1H), 7.43-7.39 (m, 2H), 7.21 (t, J=7.4 Hz, 1H), 7.09-7.07 (m, 2H), 6.70 (dd, J=2.7, 9.2 Hz, 1H), 6.46 (d, J=2.6 Hz, 1H), 3.81 (s, 3H).

### Preparation 13: 4-methoxy-2-phenoxyaniline

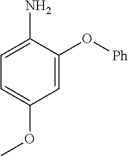 [SMILES: COc1ccc(N)c(Oc2ccccc2)c1]

Following the procedure in preparation 8, 4-methoxy-1-nitro-2-phenoxybenzene (525 mg, 2.14 mmol) was reacted to give the title compound (366 mg, 71%).

¹H NMR (500 MHz, CDCl₃) δ 7.35-7.31 (m, 2H), 7.08 (t, J=7.4 Hz, 1H), 7.02-7.00 (m, 2H), 6.71-6.69 (m, 2H), 6.60 (dd, J=2.5, 6.9 Hz, 1H), 3.92 (s, 3H), 3.90 (bs, 2H).

### Preparation 14: 1-iodo-4-methoxy-2-phenoxybenzene

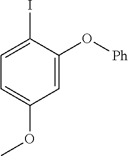 [SMILES: COc1ccc(I)c(Oc2ccccc2)c1]

Following the procedure in preparation 9, 4-methoxy-2-phenoxyaniline (366 mg, 1.70 mmol) was reacted to give the title compound (197 mg, 35%).

¹H NMR (400 MHz, CDCl₃) δ 7.38-7.34 (m, 2H), 7.29-7.23 (m, 1H), 7.14 (t, J=7.4 Hz, 1H), 7.01 (d, J=7.7 Hz, 2H), 6.63 (d, J=8.3 Hz, 1H), 6.55 (d, J=8.3 Hz, 1H), 3.96 (s, 3H).

### Preparation 15: 4-(4-methoxy-2-phenoxyphenyl)-6-methyl-1,6-dihydro-7H-pyrrolo[2,3-c]pyridin-7-one

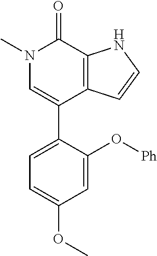 [SMILES: COc1ccc(-c2cn(C)c(=O)c3[nH]ccc23)c(Oc2ccccc2)c1]

Following the procedure in preparation 10, 1-iodo-4-methoxy-2-phenoxybenzene (198 mg, 0.61 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (260 mg, 0.61 mmol) was reacted to give the title compound (182 mg, 86%).

HPLC t_{R} (Agilent, acidic, 3.5 min): 1.59 min, m/z=347.2 [M+H]⁺.

### Preparation 16: 4-(4-hydroxy-2-phenoxyphenyl)-6-methyl-1,6-dihydro-7H-pyrrolo[2,3-c]pyridin-7-one

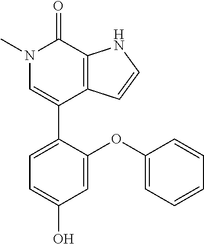 [SMILES: Cn1cc(-c2ccc(O)cc2Oc2ccccc2)c2cc[nH]c2c1=O]

Following the procedure in preparation 11, 4-(4-methoxy-2-phenoxyphenyl)-6-methyl-1,6-dihydro-7H-pyrrolo[2,3-c]pyridin-7-one (94 mg, 0.27 mmol) was reacted to give the title compound (41 mg, 43%).

HPLC t_{R} (Agilent, acidic, 3.5 min): 1.48 min, m/z=333.2 [M+H]⁺.

¹H NMR (500 MHz, DMSO-d₆) δ 11.82 (s, 1H), 9.89 (bs, 1H), 7.25 (dd, J=7.4, 8.6 Hz, 2H), 7.18-7.12 (m, 2H), 7.00 (s, 2H), 6.84 (d, J=7.6 Hz, 2H), 6.75 (d, J=7.8 Hz, 1H), 6.35 (d, J=8.1 Hz, 1H), 6.00 (d, J=2.6 Hz, 1H), 3.46 (s, 3H).

### Example 3: 4-(2-hydroxy-6-phenoxyphenyl)-6-methyl-1,6-dihydro-7H-pyrrolo[2,3-c]pyridin-7-one

### Preparation 17: 1-fluoro-3-methoxy-2-nitrobenzene

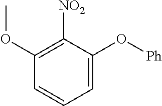 [SMILES: COc1cccc(Oc2ccccc2)c1[N+](=O)[O-]]

Following the procedure in preparation 7, 1-fluoro-3-methoxy-2-nitrobenzene (400 mg, 2.34 mmol) was reacted to give the title compound (565 mg, 89%).

¹H NMR (500 MHz, CDCl₃) δ 7.41-7.37 (m, 2H), 7.30 (dd, J=7.4, 7.4 Hz, 1H), 7.21 (dd, J=7.4, 7.4 Hz, 1H), 7.11-7.09 (m, 2H), 6.76 (d, J=7.8 Hz, 1H), 6.53 (d, J=8.5 Hz, 1H), 3.95 (s, 3H).

### Preparation 18: 2-methoxy-6-phenoxyaniline

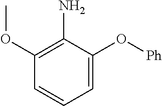 [SMILES: COc1cccc(Oc2ccccc2)c1N]

Following the procedure in preparation 8, 1-methoxy-2-nitro-3-phenoxybenzene (565 mg, 2.30 mmol) was reacted to give the title compound (350 mg, 64%).

¹H NMR (500 MHz, CDCl₃) δ 7.36-7.32 (m, 2H), 7.09 (dd, J=7.3, 7.3 Hz, 1H), 7.02-7.00 (m, 2H), 6.80 (d, J=8.7 Hz, 1H), 6.62 (dd, J=2.7, 8.7 Hz, 1H), 6.52 (d, J=2.7 Hz, 1H), 3.72 (s, 3H), 3.61-3.51 (m, 2H).

### Preparation 19: 2-iodo-1-methoxy-3-phenoxybenzene

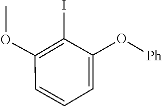 [SMILES: COc1cccc(Oc2ccccc2)c1I]

Following the procedure in preparation 9, 2-methoxy-6-phenoxyaniline (350 mg, 1.63 mmol) was reacted to give the title compound (324 mg, 55%).

¹H NMR (400 MHz, CDCl₃) δ 7.63 (d, J=8.6 Hz, 1H), 7.29-7.25 (m, 2H), 7.05 (dd, J=7.4, 7.4 Hz, 1H), 6.91 (d, J=7.6 Hz, 2H) 6.45-6.39, (m, 2H), 3.64 (s, 3H).

### Preparation 20: 4-(2-methoxy-6-phenoxyphenyl)-6-methyl-1,6-dihydro-7H-pyrrolo[2,3-c]pyridin-7-one

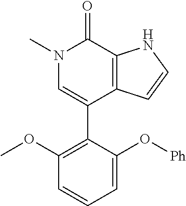 [SMILES: COc1cccc(Oc2ccccc2)c1-c1cn(C)c(=O)c2[nH]ccc12]

Following the procedure in preparation 10, 2-iodo-1-methoxy-3-phenoxybenzene (320 mg, 0.98 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (420 mg, 0.98 mmol) was reacted to give the title compound (143 mg, 42%).

HPLC t_{R} (Agilent, acidic, 3.5 min): 1.65 min, m/z=347.2 [M+H]⁺.

### Preparation 21: 4-(2-hydroxy-6-phenoxyphenyl)-6-methyl-1,6-dihydro-7H-pyrrolo[2,3-c]pyridin-7-one

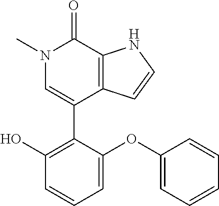 [SMILES: Cn1cc(-c2c(O)cccc2Oc2ccccc2)c2cc[nH]c2c1=O]

Following the procedure in preparation 12, 4-(2-methoxy-6-phenoxyphenyl)-6-methyl-1,6-dihydro-7H-pyrrolo[2,3-c]pyridin-7-one (190 mg, 0.55 mmol) was reacted to give the title compound (32 mg, 16%).

HPLC t_{R} (Agilent, acidic, 3.5 min): 1.40 min, m/z=333.2 [M+H]⁺.

¹H NMR (500 MHz, DMSO-d₆) δ 11.93 (d, J=0.9 Hz, 1H), 9.64 (s, 1H), 7.33-7.22 (m, 4H), 7.13 (s, 1H), 7.06 (t, J=7.4 Hz, 1H), 6.93 (d, J=7.8 Hz, 2H), 6.64 (dd, J=2.4, 8.3 Hz, 1H), 6.35 (d, J=2.4 Hz, 1H), 6.22-6.21 (m, 1H), 3.49 (s, 3H).

### Example 4: 6-methyl-4-(4-phenoxythiazol-5-yl)-1,6-dihydro-7H-pyrrolo[2,3-c]pyridin-7-one

### Preparation 22: 4-phenoxythiazole

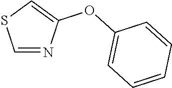 [SMILES: c1ccc(Oc2cscn2)cc1]

To an oven dried microwave vial was added CuI (58.0 mg, 0.30 mmol), picolinic acid (75.0 mg, 0.61 mmol), phenol (0.31 mL, 3.66 mmol) and potassium phosphate tribasic (1.3 g, 6.1 mmol). The tube was then evacuated and backfilled with N₂ twice. 4-bromo-thiazole (500 mg, 3.0 mmol) in DMSO (10 mL) was added and the mixture heated at 150 °C for 1 hr. DMSO was removed using a Genevac EZ-2. To the residue was added ethyl acetate (50 mL) and the organics washed with 2×50 mL water then 1×50 mL saturated brine solution. The organics were then separated and dried (MgSO₄) before concentration to dryness. The crude product was purified by flash chromatography on silica gel eluting with ethyl acetate/heptane gradient (0-100%). Fractions corresponding to product were combined and concentrated to give the title compound, a yellow solid (93 mg, 17%).

HPLC t_{R} (Agilent, acidic, 3.5 min): 1.54 min, m/z=177.9 [M+H]⁺.

### Preparation 23: 5-bromo-4-phenoxythiazole

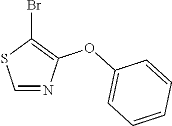 [SMILES: Brc1scnc1Oc1ccccc1]

4-phenoxythiazole (60 mg, 0.34 mmol) in MeCN (2 mL) was cooled to 0 °C. 1-bromopyrrolidine-2,5-dione (72 mg, 0.41 mmol) in MeCN (2 mL) was added dropwise. The reaction was warmed to RT and left for 3 h. Ethyl acetate (50 ml) was added and the organics washed with 2×50 ml saturated sodium carbonate then 1×50 ml saturated brine solution. The organics were then separated and dried (MgSO₄) before concentration to dryness to give the title compound (75 mg, 87%). The material was used directly in preparation 27 without further purification. HPLC t_{R} (Agilent, acidic, 3.5 min): 1.71 min, m/z=257.7 [M+H]⁺.

### Preparation 24: 6-methyl-4-(4-phenoxythiazol-5-yl)-1,6-dihydro-7H-pyrrolo[2,3-c]pyridin-7-one

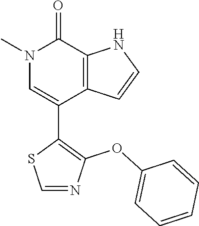 [SMILES: Cn1cc(-c2scnc2Oc2ccccc2)c2cc[nH]c2c1=O]

Following the procedure in preparation 10, 5-bromo-4-phenoxythiazole (72 mg, 0.28 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (110 mg, 0.26 mmol) was reacted to give the title compound (10 mg, 12%).

HPLC t_{R} (Agilent, acidic, 3.5 min): 1.43 min, m/z=324.2 [M+H]⁺.

¹H NMR (500 MHz, DMSO-d₆) δ 12.20 (bs, 1H), 9.02 (s, 1H), 7.52 (s, 1H), 7.36-7.31 (m, 3H), 7.09 (t, J=7.4 Hz, 1H), 7.00 (d, J=7.9 Hz, 2H), 6.44 (s, 1H), 3.53 (s, 3H).

### Example 5: 6-methyl-4-(1-methyl-2-oxo-3-phenoxy-1,2-dihydropyridin-4-yl)-1,6-dihydro-7H-pyrrolo[2,3-c]pyridin-7-one

### Preparation 25: 4-nitro-3-phenoxypyridine 1-oxide

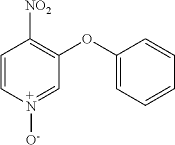

Sodium hydride (278 mg, 6.96 mmol) in DMF (5 mL) was cooled to 0 °C. Phenol (0.58 mL, 6.96 mmol) in DMF (5 mL) was added and the mixture stirred for 10 min at this temperature before 3-fluoro-4-nitro-pyridine 1-oxide (1.0 g, 6.3 mmol) in DMF (5 mL) was added. The reaction was warmed to RT over 30 min. The reaction mixture was stirred with ice-water and extracted with DCM. The organic extract was washed with water and saturated sodium chloride solution, dried (MgSO₄), filtered and concentrated to give 4-nitro-3-phenoxy-pyridine 1-oxide (1.1 g, 74.9%), as a waxy solid. The material was used directly in preparation 29 without further purification.

HPLC t_{R} (Agilent, acidic, 3.5 min): 1.72 min, m/z=233.4 [M+H]⁺.

### Preparation 26: 4-bromo-3-phenoxypyridine 1-oxide

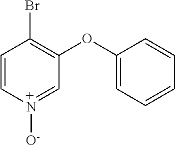

4-nitro-3-phenoxy-pyridine 1-oxide (1.1 g, 4.74 mmol) in acetyl bromide (3.51 mL, 47.4 mmol) was stirred at reflux for 2 hr. After cooling to ambient temperature the mixture was poured onto crushed ice and stirred vigorously. The solution was brought to pH 10 with careful addition of saturated sodium carbonate. The organic extract was washed with water and saturated sodium chloride solution, dried (MgSO₄), filtered and concentrated. Purification was performed by silica gel chromatography, eluting with ethyl acetate/heptane gradient (0-100%) to give the title product 4-bromo-3-phenoxy-pyridine 1-oxide (870 mg, 62%).

HPLC t_{R} (Agilent, acidic, 3.5 min): 1.55 min, m/z=267.9 [M+H]⁺.

### Preparation 27: 4-bromo-3-phenoxypyridine

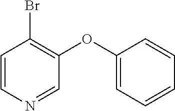 [SMILES: Brc1ccncc1Oc1ccccc1]

4-bromo-3-phenoxy-pyridine 1-oxide (715 mg, 2.69 mmol) in chloroform (20 mL) was cooled to 0 °C. PBr₃ (1.2 g, 3.23 mmol) in chloroform (20 mL) was added dropwise and then warmed to 50 °C for 1 hr. The reaction was concentrated to dryness and the residue was taken up in ethyl acetate (50 mL) and the organics washed with 2×50 mL water then 1×50 mL saturated brine solution. The organics were then separated and dried (MgSO₄) before concentration to dryness. The crude mixture was then purified by flash column chromatography eluting with ethyl acetate/heptane gradient (0-100%). The desired fractions were concentrated to dryness in vacuo to give 4-bromo-3-phenoxy-pyridine (500 mg, 74%).

HPLC t_{R} (Agilent, acidic, 3.5 min): 1.61 min, m/z=249.8 [M]⁺.

### Preparation 28: 4-bromo-1-methyl-3-phenoxypyridin-2 (1H)-one

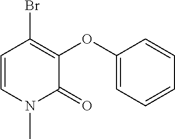 [SMILES: Cn1ccc(Br)c(Oc2ccccc2)c1=O]

In a sealable tube, to 4-bromo-3-phenoxy-pyridine (528 mg, 2.11 mmol) in MeCN (10 mL) was added dimethyl sulfate (2.0 mL, 21.1 mmol) and the mixture heated at 80 °C for 30 min. Additional dimethyl sulfate (7.0 mL, 73.9 mmol) was added and the mixture heated at 80 °C for 16 hr. The mixture was cooled on ice to 0 °C and potassium ferricyanide (1.74 g, 5.28 mmol) in water (5 mL) was added followed by dropwise addition of potassium hydroxide (948 mg, 16.9 mmol) in water (5 mL) and stirred at 80 °C for 16 hr. DCM and water were added and passed through a phase separator. The organic layer was dried and the residue purified by column chromatography, eluting with ethyl acetate/heptane gradient (0-100%). The desired fractions were concentrated to dryness in vacuo to give 4-bromo-1-methyl-5-phenoxypyridin-2 (1H)-one (45 mg, 7.6%).

HPLC t_{R} (Agilent, acidic, 3.5 min): 1.42 min, m/z=281.0 [M+H]⁺.

### Preparation 29: 6-methyl-4-(1-methyl-2-oxo-3-phenoxy-1,2-dihydropyridin-4-yl)-1,6-dihydro-7H-pyrrolo[2,3-c]pyridin-7-one

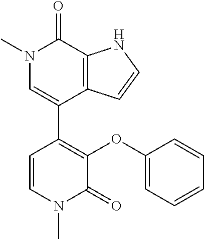 [SMILES: Cn1ccc(-c2cn(C)c(=O)c3[nH]ccc23)c(Oc2ccccc2)c1=O]

Following the procedure in preparation 10, 4-bromo-1-methyl-5-phenoxypyridin-2 (1H)-one (45 mg, 0.16 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (65 mg, 0.15 mmol) was reacted to give the title compound (6 mg, 11%).

HPLC t_{R} (Agilent, acidic, 3.5 min): 1.17 min, m/z=348.1 [M+H]⁺.

¹H NMR (500 MHz, DMSO-d₆) δ 12.08 (bs, 1H), 7.71 (d, J=7.0 Hz, 1H), 7.36-7.30 (m, 2H), 7.18 (dd, J=7.9, 7.9 Hz, 2H), 6.90 (dd, J=7.4, 7.4 Hz, 1H), 6.71 (d, J=7.8 Hz, 2H), 6.41 (d, J=7.0 Hz, 1H), 6.31 (dd, J=2.3, 2.3 Hz, 1H), 3.53 (s, 3H), 3.47 (s, 3H).

### Example 6: 4-(5-hydroxy-2-phenoxyphenyl)-6-methyl-1,6-dihydro-7H-pyrrolo[2,3-c]pyridin-7-one

### Preparation 30: 4-methoxy-2-nitro-1-phenoxybenzene

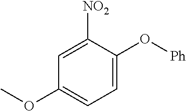 [SMILES: COc1ccc(Oc2ccccc2)c([N+](=O)[O-])c1]

Following the procedure in preparation 7, 1-fluoro-4-methoxy-2-nitrobenzene (1.2 g, 7.01 mmol) was reacted to give the title compound (1.50 mg, 87%).

HPLC t_{R} (Agilent, acidic, 3.5 min): 1.79 min, m/z=246.1 [M+H]⁺.

### Preparation 31: 5-methoxy-2-phenoxyaniline

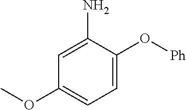 [SMILES: COc1ccc(Oc2ccccc2)c(N)c1]

Following the procedure in preparation 8, 4-methoxy-2-nitro-1-phenoxybenzene (1.80 mg, 5.38 mmol) was reacted to give the title compound (1.23 g, 64%).

HPLC t_{R} (Agilent, acidic, 3.5 min): 1.64 min, m/z=216.1 [M+H]⁺.

### Preparation 32: 2-iodo-4-methoxy-1-phenoxybenzene

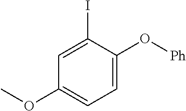 [SMILES: COc1ccc(Oc2ccccc2)c(I)c1]

Following the procedure in preparation 9, 5-methoxy-2-phenoxyaniline (1.23 g, 5.71 mmol) was reacted to give the title compound (180 mg, 10%).

¹H NMR (500 MHz, CDCl₃) 7.41 (d, J=2.9 Hz, 1H), 7.35-7.31 (m, 2H), 7.10-7.03 (m, 1H), 6.96-6.91 (m, 4H), 3.83 (s, 3H).

### Preparation 33: 4-(5-methoxy-2-phenoxyphenyl)-6-methyl-1,6-dihydro-7H-pyrrolo[2,3-c]pyridin-7-one

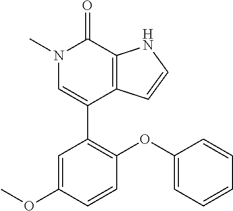 [SMILES: COc1ccc(Oc2ccccc2)c(-c2cn(C)c(=O)c3[nH]ccc23)c1]

Following the procedure in preparation 10, 2-iodo-4-methoxy-1-phenoxybenzene (152 mg, 0.47 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (200 mg, 0.47 mmol) was reacted to give the title compound (88 mg, 39%).

HPLC t_{R} (Agilent, acidic, 3.5 min): 1.65 min, m/z=347.2 [M+H]⁺.

### Preparation 34: 4-(5-hydroxy-2-phenoxyphenyl)-6-methyl-1,6-dihydro-7H-pyrrolo[2,3-c]pyridin-7-one

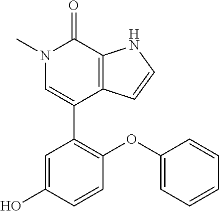 [SMILES: Cn1cc(-c2cc(O)ccc2Oc2ccccc2)c2cc[nH]c2c1=O]

Following the procedure in preparation 11, 4-(5-methoxy-2-phenoxyphenyl)-6-methyl-1,6-dihydro-7H-pyrrolo[2,3-c]pyridin-7-one (85 mg, 0.25 mmol) was reacted to give the title compound (50 mg, 58%).

HPLC t_{R} (Agilent, acidic, 3.5 min): 1.46 min, m/z=333.2 [M+H]⁺.

¹H NMR (500 MHz, DMSO-d₆) d 11.97 (s, 1H), 9.47 (s, 1H), 7.26 (dd, J=2.7, 2.7 Hz, 1H), 7.21-7.17 (m, 3H), 6.96-6.89 (m, 3H), 6.79 (dd, J=2.9, 8.7 Hz, 1H), 6.73 (d, J=7.6 Hz, 2H), 6.25-6.23 (m, 1H), 3.46 (s, 3H).

### Example 7: 6-methyl-4-(1-methyl-2-oxo-5-phenoxy-1,2-dihydropyridin-4-yl)-1,6-dihydro-7H-pyrrolo[2,3-c]pyridin-7-one

### Preparation 35: 2-chloro-4-nitro-5-phenoxypyridine 1-oxide

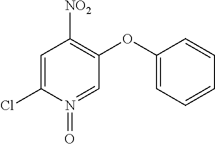 [SMILES: O=[N+]([O-])c1cc(Cl)[n+]([O-])cc1Oc1ccccc1]

To a solution of 2-chloro-5-fluoro-4-nitropyridine 1-oxide (2.00 g, 10.4 mmol) in THF (100 mL) was added K₂CO₃ (2.87 g, 20.8 mmol); phenol (1.03 g, 10.9 mmol) at 20 °C, the reaction was stirred at 90 °C for 1 hour. The reaction was concentrated in vacuum and the residue was diluted with saturated NaHCO₃ (100 mL), the reaction mixture was extracted with DCM (100 mL×2), The combined organic phase was washed with brine (100 mL), dried with anhydrous Na₂SO₄, filtered and concentrated in vacuum. The residue was purified by column chromatography (SiO₂, Petroleum ether/Ethyl acetate=10/1 to 3/1) to give the title compound (950 mg, 3.56 mmol, yield=34.3%) was obtained as a yellow solid.

HPLC t_{R} (Agilent, acidic, 3.5 min): 1.47 min, m/z=267.0 [M+H]⁺.

### Preparation 36: 2,4-dibromo-5-phenoxypyridine 1-oxide

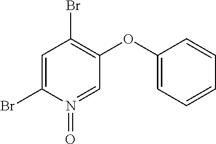 [SMILES: [O-][n+]1cc(Oc2ccccc2)c(Br)cc1Br]

Following the procedure in preparation 26, 2-chloro-4-nitro-5-phenoxypyridine 1-oxide (950 mg, 3.56 mmol) was reacted to give the title compound (1.2 g, 98%).

HPLC t_{R} (Agilent, acidic, 3.5 min): 1.49 min, m/z=345.9 [M+H]⁺.

### Preparation 37: 2,4-dibromo-5-phenoxypyridine

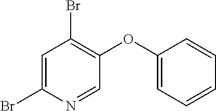 [SMILES: Brc1cc(Br)c(Oc2ccccc2)cn1]

Following the procedure in preparation 27, 2,4-dibromo-5-phenoxypyridine 1-oxide (1.3 g, 3.77 mmol) was reacted to give the title compound (1.1 g, 89%).

HPLC t_{R} (Agilent, acidic, 3.5 min): 1.91 min, m/z=330.0 [M+H]⁺.

### Preparation 38: 4-bromo-5-phenoxypyridin-2 (1H)-one

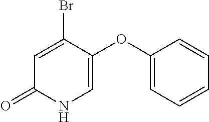 [SMILES: O=c1cc(Br)c(Oc2ccccc2)c[nH]1]

To a solution of 2,4-dibromo-5-phenoxypyridine (1.28 g, 3.9 mmol) in t-BuOH (30 mL) was added KOH (699 mg, 12.5 mmol) at 20 °C, the reaction mixture was stirred at 90 °C for 12 hours. The reaction was concentrated in vacuum. The residue was diluted with H₂O (100 mL) and extracted with DCM (100 mL×2), the combined organic phase was washed with brine (100 mL), dried with anhydrous Na₂SO₄, filtered and concentrated in vacuum. The residue was purified by prep-HPLC (TFA condition) to give the title compound (80 mg, 0.3 mmol, yield=7.8%) as a yellow solid.

HPLC t_{R} (Agilent, acidic, 3.5 min): 1.39 min, m/z=267.1 [M+H]⁺.

### Preparation 39: 4-bromo-1-methyl-5-phenoxypyridin-2 (1H)-one

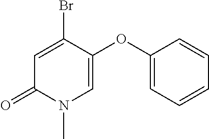 [SMILES: Cn1cc(Oc2ccccc2)c(Br)cc1=O]

To a solution of 4-bromo-5-phenoxypyridin-2 (1H)-one (61 mg, 0.23 mmol) in DMF (3.0 mL) was added MeI (65.1 mg, 0.46 mmol, 2.52 mL); Cs₂CO₃ (224.1 mg, 0.69 mmol) at 20 °C, the reaction was stirred at 20 °C for 1 hours. To this reaction was added H₂O (100 mL) and extracted with DCM (100 mL×2), the combined organic phase was washed with brine (100 mL), dried with anhydrous Na₂SO₄, filtered and concentrated in vacuum. The residue was purified by prep-HPLC (TFA condition) to give the 4-bromo-1-methyl-5-phenoxypyridin-2 (1H)-one (63 mg, 0.23 mmol, yield=98%) as a yellow solid.

HPLC t_{R} (Agilent, acidic, 3.5 min): 1.45 min, m/z=281.0 [M+H]⁺.

### Preparation 40: 6-methyl-4-(1-methyl-2-oxo-5-phenoxy-1,2-dihydropyridin-4-yl)-1,6-dihydro-7H-pyrrolo[2,3-c]pyridin-7-one

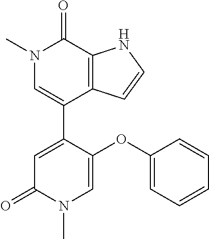 [SMILES: Cn1cc(Oc2ccccc2)c(-c2cn(C)c(=O)c3[nH]ccc23)cc1=O]

Following the procedure in preparation 10, 4-bromo-1-methyl-5-phenoxypyridin-2 (1H)-one (65 mg, 0.23 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (99 mg, 0.23 mmol) was reacted to give the title compound (23 mg, 24%).

HPLC t_{R} (Agilent, acidic, 3.5 min): 1.26 min, m/z=348.2 [M+H]⁺.

¹H NMR (500 MHz, DMSO-d₆) δ 12.04 (bs, 1H), 7.86 (s, 1H), 7.37 (s, 1H), 7.29 (t, J=2.8 Hz, 1H), 7.19-7.14 (m, 2H), 6.89 (t, J=7.4 Hz, 1H), 6.79-6.76 (m, 2H), 6.54 (s, 1H), 6.34 (t, J=2.4 Hz, 1H), 3.48 (s, 3H), 3.45 (s, 3H).

### Example 8: 5-(5-hydroxy-2-phenoxyphenyl)-1-methylpyridin-2 (1H)-one

### Preparation 41: 5-(5-methoxy-2-phenoxyphenyl)-1-methylpyridin-2 (1H)-one

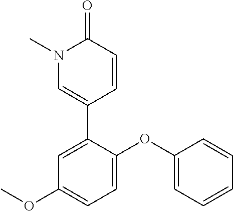 [SMILES: COc1ccc(Oc2ccccc2)c(-c2ccc(=O)n(C)c2)c1]

In a microwave tube, 2-iodo-4-methoxy-1-phenoxybenzene (90 mg, 0.28 mmol), sodium carbonate (81.6 mg, 0.77 mmol) and 1-methyl-5-(4,4,5,5-tetramethyl-1,3,2-dioxaborolan-2-yl)pyridin-2 (1H)-one (84 mg, 0.36 mmol) in 1,2-Dimethoxyethane (2 mL) and Water (1 mL) was degassed by bubbling nitrogen for 10 min. Pd(PPh₃)₄ (14.83 mg, 0.013 mmol) was added, the tube sealed and the reaction heated at 120 °C for 30 min. Ethyl acetate (50 ml) was added and the organics washed with 2×50 ml water then 1×50 ml saturated brine solution. The organics were then separated and dried (MgSO₄) before concentration to dryness. The crude was then purified by flash column chromatography eluting with ethyl acetate/heptane gradient (0-100%). The desired fractions were combined and dried to give the title compound (45 mg, 48%) as a white solid.

HPLC t_{R} (Agilent, acidic, 3.5 min): 1.58 min, m/z=308.2 [M+H]⁺.

### Preparation 42: 5-(5-hydroxy-2-phenoxyphenyl)-1-methylpyridin-2 (1H)-one

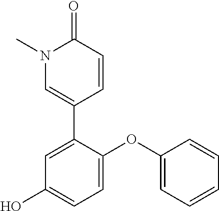 [SMILES: Cn1cc(-c2cc(O)ccc2Oc2ccccc2)ccc1=O]

Following the procedure in preparation 11, 5-(5-methoxy-2-phenoxyphenyl)-1-methylpyridin-2 (1H)-one (45 mg, 0.15 mmol) was reacted to give the title compound (26 mg, 55%).

HPLC t_{R} (Agilent, acidic, 3.5 min): 1.43 min, m/z=294.2 [M+H]⁺.

¹H NMR (500 MHz, DMSO-d₆) δ 9.50 (s, 1H), 7.85 (d, J=2.4 Hz, 1H), 7.55-7.53 (m, 1H), 7.29-7.25 (m, 2H), 7.00-6.90 (m, 2H), 6.85-6.76 (m, 4H), 6.33 (d, J=9.5 Hz, 1H), 3.42 (s, 3H).

### Example 9: 4-(5-hydroxy-2-propoxyphenyl)-6-methyl-1,6-dihydro-7H-pyrrolo[2,3-c]pyridin-7-one

### Preparation 43: 1-fluoro-4-((4-methoxybenzyl)oxy)-2-nitrobenzene

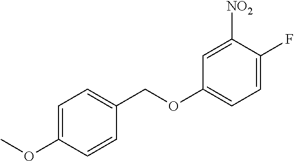 [SMILES: COc1ccc(COc2ccc(F)c([N+](=O)[O-])c2)cc1]

To a solution of 4-fluoro-3-nitrophenol (2.30 g, 14.6 mmol) in DMF (20.0 mL) was added potassium tert-butoxide (1.97 g, 17.6 mmol) at 20 °C and stirred for 15 minutes. 1-(chloromethyl)-4-methoxybenzene (2.7 mL, 19.0 mmol) was added and the reaction was stirred at 20 °C for 1.5 hours. The reaction was concentrated under vacuum. The residue was dissolved in EtOAc (2×400 mL) and washed with H₂O (300 mL), the combined organic phase was washed with brine (100 mL), dried with anhydrous Na₂SO₄, filtered and concentrated in vacuum to give 1-fluoro-4-((4-methoxybenzyl)oxy)-2-nitrobenzene (2.52 g, 8.64 mmol, yield=59%) as a yellow solid.

¹H NMR (400 MHz, CDCl₃) δ 7.63-7.60 (m, 1H), 7.36-7.33 (m, 2H), 7.22-7.18 (m, 2H), 6.96-6.91 (m, 2H), 5.02 (s, 2H), 3.83 (s, 3H).

### Preparation 44: 4-((4-methoxybenzyl)oxy)-2-nitro-1-propoxybenzene

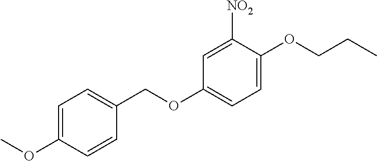 [SMILES: CCCOc1ccc(OCc2ccc(OC)cc2)cc1[N+](=O)[O-]]

To a solution of 1-fluoro-4-((4-methoxybenzyl)oxy)-2-nitrobenzene (750 mg, 2.7 mmol) in DMF (10 mL) was added sodium hydride (194 mg, 8.1 mmol) at 20 °C and stirred for 30 minutes. 1-propanol (0.6 mL, 8.1 mmol) was added and the reaction was stirred at 20 °C for 20 minutes. The reaction was concentrated under vacuum. The residue was dissolved in EtOAc (2×100 mL) and washed with H₂O (100 mL), the combined organic phase was washed with brine (100 mL), dried with anhydrous Na₂SO₄, filtered and concentrated in vacuum to give 4-((4-methoxybenzyl)oxy)-2-nitro-1-propoxybenzene (710 mg, 2.13 mmol, yield=79%) as a yellow solid.

¹H NMR (500 MHz, CDCl₃) δ 7.45-7.44 (m, 1H), 7.36-7.32 (m, 2H), 7.13 (dd, J=3.2, 9.2 Hz, 1H), 7.01-6.91 (m, 3H), 4.98 (s, 2H), 4.01 (t, J=6.4 Hz, 2H), 3.83 (s, 3H), 1.84 (tt, J=8.2, 8.8 Hz, 2H), 1.05 (t, J=7.6 Hz, 3H).

### Preparation 45: 5-((4-methoxybenzyl)oxy)-2-propoxyaniline

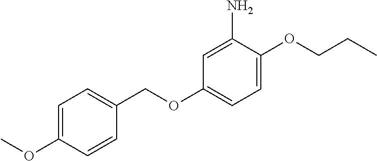 [SMILES: CCCOc1ccc(OCc2ccc(OC)cc2)cc1N]

Following the procedure in preparation 8, 4-((4-methoxybenzyl)oxy)-2-nitro-1-propoxybenzene (710 mg, 2.23 mmol) was reacted to give the title compound (495 mg, 73%). HPLC t_{R} (Agilent, acidic, 3.5 min): 1.73 min, m/z=288.2 [M+H]⁺.

### Preparation 46: 2-iodo-4-((4-methoxybenzyl)oxy)-1-propoxybenzene

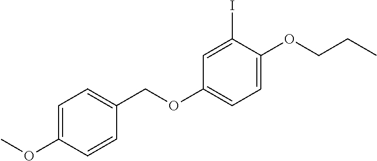 [SMILES: CCCOc1ccc(OCc2ccc(OC)cc2)cc1I]

Following the procedure in preparation 9, 5-((4-methoxybenzyl)oxy)-2-propoxyaniline (495 mg, 1.73 mmol) was reacted to give the title compound (360 mg, 50%).

¹H NMR (500 MHz, DMSO-d₆) δ 7.41 (d, J=2.9 Hz, 1H), 7.34-7.31 (m, 2H), 6.92-6.87 (m, 3H), 6.72 (d, J=9.6 Hz, 1H), 4.91 (s, 2H), 3.90 (t, J=6.4 Hz, 2H), 3.81 (s, 3H), 1.82 (tdt, J=6.7, 6.7, 6.8 Hz, 2H), 1.07 (t, J=7.4 Hz, 3H).

### Preparation 47: 4-(5-((4-methoxybenzyl)oxy)-2-propoxyphenyl)-6-methyl-1-tosyl-1,6-dihydro-7H-pyrrolo[2,3-c]pyridin-7-one

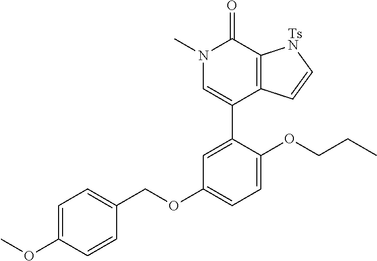 [SMILES: CCCOc1ccc(OCc2ccc(OC)cc2)cc1-c1cn(C)c(=O)c2c1ccn2S(=O)(=O)c1ccc(C)cc1]

Following the procedure in preparation 40, 2-iodo-4-((4-methoxybenzyl)oxy)-1-propoxybenzene (107 mg, 0.27 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (115 mg, 0.27 mmol) was reacted to give the title compound (93 mg, 54%).

HPLC t_{R} (Agilent, acidic, 3.5 min): 2.07 min, m/z=573.3 [M+H]⁺.

### Preparation 48: 4-(5-hydroxy-2-propoxyphenyl)-6-methyl-1-tosyl-1,6-dihydro-7H-pyrrolo[2,3-c]pyridin-7-one

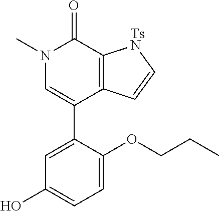 [SMILES: CCCOc1ccc(O)cc1-c1cn(C)c(=O)c2c1ccn2S(=O)(=O)c1ccc(C)cc1]

To a solution of 4-(5-((4-methoxybenzyl)oxy)-2-propoxyphenyl)-6-methyl-1-tosyl-1,6-dihydro-7H-pyrrolo[2,3-c]pyridin-7-one (90 mg, 0.16 mmol) in DCM (2 mL) was added trifluoroacetic acid (0.072 mL, 0.94 mmol) at 20 °C and stirred for 4 hours. The reaction was concentrated under vacuum. The residue was dissolved in EtOAc (2×100 mL) and washed with H₂O (100 mL), the combined organic phase was washed with brine (100 mL), dried with anhydrous Na₂SO₄, filtered and concentrated in vacuum to give 4-((4-methoxybenzyl)oxy)-2-nitro-1-propoxybenzene (55 mg, 0.12 mmol, yield=62%) as a yellow solid.

HPLC t_{R} (Agilent, acidic, 3.5 min): 1.72 min, m/z=453.2 [M+H]⁺.

### Preparation 49: 4-(5-hydroxy-2-propoxyphenyl)-6-methyl-1,6-dihydro-7H-pyrrolo[2,3-c]pyridin-7-one

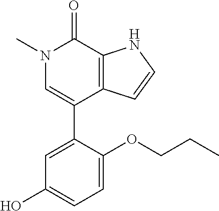 [SMILES: CCCOc1ccc(O)cc1-c1cn(C)c(=O)c2[nH]ccc12]

To a solution of 4-(5-hydroxy-2-propoxyphenyl)-6-methyl-1-tosyl-1,6-dihydro-7H-pyrrolo[2,3-c]pyridin-7-one (55 mg, 0.12 mmol) in THF (1 mL) and methanol (1 mL) was added sodium hydroxide (25.5 mg, 0.61 mmol) and the reaction mixture heated to 60 °C and stirred for 4 hours. The reaction was concentrated under vacuum. The residue was dissolved in EtOAc (2×100 mL) and washed with H₂O (100 mL), the combined organic phase was washed with brine (100 mL), dried with anhydrous Na₂SO₄, filtered and concentrated in vacuum. The crude material was purified by column chromatography (0-50% 20% MeOH/DCM in DCM) followed by reverse phase preparative HPLC (Gilson acidic 60-90% gradient). Fractions were concentrated on the genevac overnight to afford 4-((4-methoxybenzyl)oxy)-2-nitro-1-propoxybenzene (55 mg, 0.10 mmol, yield=62%) as a yellow solid.

HPLC t_{R} (Agilent, acidic, 3.5 min): 1.39 min, m/z=299.2 [M+H]⁺.

¹H NMR (500 MHz, CDCl₃) δ 10.21 (bs, 1H), 7.22 (s, 1H), 7.16 (t, J=2.8 Hz, 1H), 7.06 (s, 1H), 6.92-6.85 (m, 3H), 6.28 (t, J=2.5 Hz, 1H), 3.80 (t, J=6.5 Hz, 2H), 3.67 (s, 3H), 1.61 (dt, J=7.8, 13.9 Hz, 2H), 0.85 (t, J=7.8 Hz, 3H).

### Example 10: 4-(2-cyclobutoxy-5-hydroxyphenyl)-6-methyl-1,6-dihydro-7H-pyrrolo[2,3-c]pyridin-7-one

### Preparation 50: 1-cyclobutoxy-4-((4-methoxybenzyl)oxy)-2-nitrobenzene

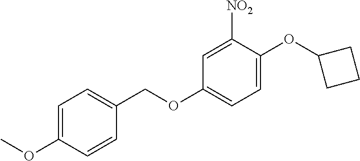 [SMILES: COc1ccc(COc2ccc(OC3CCC3)c([N+](=O)[O-])c2)cc1]

Following the procedure in preparation 44, 1-fluoro-4-((4-methoxybenzyl)oxy)-2-nitrobenzene (750 mg, 2.70 mmol) and cyclobutanol (0.64 mL, 9.2 mmol) was reacted to give the title compound (601 mg, 64%).

¹H NMR (400 MHz, CDCl₃) δ 7.44 (d, J=3.1 Hz, 1H), 7.34-7.31 (m, 2H), 7.09 (dd, J=3.2, 9.1 Hz, 1H), 6.93-6.83 (m, 3H), 4.96 (s, 2H), 4.71-4.64 (m, 1H), 3.82 (s, 3H), 2.47-2.39 (m, 2H), 2.29-2.18 (m, 2H), 1.91-1.83 (m, 1H), 1.73-1.61 (m, 1H).

### Preparation 51: 2-cyclobutoxy-5-((4-methoxybenzyl)oxy) aniline

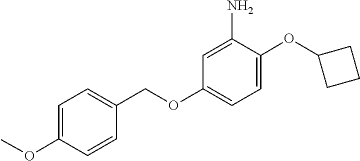 [SMILES: COc1ccc(COc2ccc(OC3CCC3)c(N)c2)cc1]

Following the procedure in preparation 44, 1-cyclobutoxy-4-((4-methoxybenzyl)oxy)-2-nitrobenzene (601 mg, 1.82 mmol) was reacted to give the title compound (375 mg, 62%).

HPLC t_{R} (Agilent, acidic, 3.5 min): 1.76 min, m/z=300.2 [M+H]⁺.

### Preparation 52: 1-cyclobutoxy-2-iodo-4-((4-methoxybenzyl)oxy)benzene

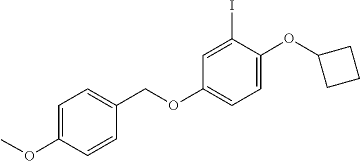 [SMILES: COc1ccc(COc2ccc(OC3CCC3)c(I)c2)cc1]

Following the procedure in preparation 45, 2-cyclobutoxy-5-((4-methoxybenzyl)oxy) aniline (375 mg, 1.25 mmol) was reacted to give the title compound (360 mg, 67%).

¹H NMR (400 MHz, CDCl₃) δ 7.40 (d, J=3.0 Hz, 1H), 7.32 (d, J=8.3 Hz, 2H), 6.92-6.84 (m, 3H), 6.60 (d, J=8.3 Hz, 1H), 4.89 (s, 2H), 4.61-4.54 (m, 1H), 3.81 (s, 3H), 2.45-2.37 (m, 2H), 2.27-2.17 (m, 2H), 1.89-1.81 (m, 1H), 1.68-1.58 (m, 1H).

### Preparation 53: 4-(2-cyclobutoxy-5-((4-methoxybenzyl)oxy)phenyl)-6-methyl-1-tosyl-1,6-dihydro-7H-pyrrolo[2,3-c]pyridin-7-one

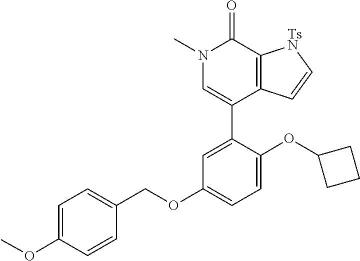 [SMILES: COc1ccc(COc2ccc(OC3CCC3)c(-c3cn(C)c(=O)c4c3ccn4S(=O)(=O)c3ccc(C)cc3)c2)cc1]

Following the procedure in preparation 40, 1-cyclobutoxy-2-iodo-4-((4-methoxybenzyl)oxy)benzene (119 mg, 0.29 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (125 mg, 0.29 mmol) was reacted to give the title compound (130 mg, 72%).

HPLC t_{R} (Agilent, acidic, 3.5 min): 2.10 min, m/z=585.2 [M+H]⁺.

### Preparation 54: 4-(2-cyclobutoxy-5-hydroxyphenyl)-6-methyl-1-tosyl-1,6-dihydro-7H-pyrrolo[2,3-c]pyridin-7-one

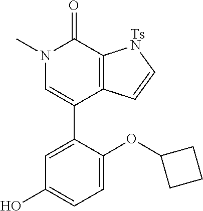 [SMILES: Cc1ccc(S(=O)(=O)n2ccc3c(-c4cc(O)ccc4OC4CCC4)cn(C)c(=O)c32)cc1]

Following the procedure in preparation 48, 4-(2-cyclobutoxy-5-((4-methoxybenzyl)oxy)phenyl)-6-methyl-1-tosyl-1,6-dihydro-7H-pyrrolo[2,3-c]pyridin-7-one (130 mg, 0.22 mmol) was reacted to give the title compound (75 mg, 58%).

HPLC t_{R} (Agilent, acidic, 3.5 min): 1.71 min, m/z=465.2 [M+H]⁺.

### Preparation 55: 4-(2-cyclobutoxy-5-hydroxyphenyl)-6-methyl-1,6-dihydro-7H-pyrrolo[2,3-c]pyridin-7-one

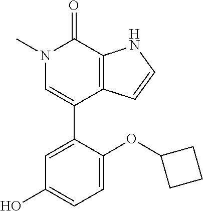 [SMILES: Cn1cc(-c2cc(O)ccc2OC2CCC2)c2cc[nH]c2c1=O]

Following the procedure in preparation 49, 4-(2-cyclobutoxy-5-hydroxyphenyl)-6-methyl-1-tosyl-1,6-dihydro-7H-pyrrolo[2,3-c]pyridin-7-one (74 mg, 0.16 mmol) was reacted to give the title compound (4 mg, 7%).

HPLC t_{R} (Agilent, acidic, 3.5 min): 1.38 min, m/z=311.2 [M+H]⁺.

¹H NMR (400 MHz, DMSO) δ 11.94 (s, 1H), 8.97 (s, 1H), 7.27-7.25 (m, 1H), 7.18 (s, 1H), 6.77-6.74 (m, 1H), 6.73 (s, 1H), 6.66 (dd, J=2.9, 8.7 Hz, 1H), 6.14-6.11 (m, 1H), 4.52-4.44 (m, 1H), 3.54 (s, 3H), 2.31-2.23 (m, 2H), 1.92-1.82 (m, 2H), 1.70-1.49 (m, 2H).

### Example 11: 4-(2-(cyclohexyloxy)-5-hydroxyphenyl)-6-methyl-1,6-dihydro-7H-pyrrolo[2,3-c]pyridin-7-one

### Preparation 56: 1-(cyclohexyloxy)-4-((4-methoxybenzyl)oxy)-2-nitrobenzene

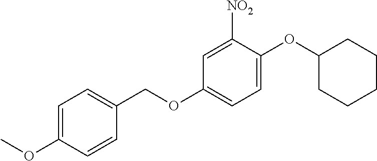 [SMILES: COc1ccc(COc2ccc(OC3CCCCC3)c([N+](=O)[O-])c2)cc1]

Following the procedure in preparation 44, 1-fluoro-4-((4-methoxybenzyl)oxy)-2-nitrobenzene (750 mg, 2.70 mmol) and cyclohexanol (0.87 mL, 8.2 mmol) was reacted to give the title compound (843 mg, 82%).

¹H NMR (400 MHz, CDCl₃) δ 7.40 (d, J=3.4 Hz, 1H), 7.35-7.31 (m, 2H), 7.10 (dd, J=3.1, 9.2 Hz, 1H), 7.02 (d, J=9.6 Hz, 1H), 6.95-6.90 (m, 2H), 4.97-4.96 (m, 2H), 4.28 (tt, J=4.2, 7.9 Hz, 1H), 3.82 (s, 3H), 1.95-1.88 (m, 2H), 1.81 (dd, J=10.2, 10.2 Hz, 2H), 1.68-1.50 (m, 3H), 1.38-1.26 (m, 3H).

### Preparation 57: 2-(cyclohexyloxy)-5-((4-methoxybenzyl)oxy) aniline

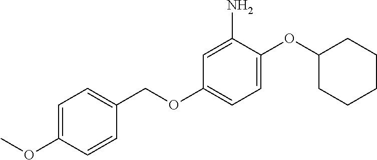 [SMILES: COc1ccc(COc2ccc(OC3CCCCC3)c(N)c2)cc1]

Following the procedure in preparation 44, 1-(cyclohexyloxy)-4-((4-methoxybenzyl)oxy)-2-nitrobenzene (843 mg, 2.35 mmol) was reacted to give the title compound (469 mg, 55%). HPLC t_{R} (Agilent, acidic, 3.5 min): 1.91 min, m/z=328.2 [M+H]⁺.

### Preparation 58: 1-(cyclohexyloxy)-2-iodo-4-((4-methoxybenzyl)oxy)benzene

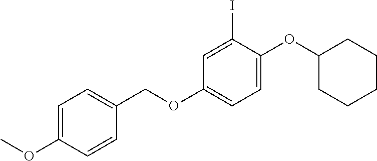 [SMILES: COc1ccc(COc2ccc(OC3CCCCC3)c(I)c2)cc1]

Following the procedure in preparation 45, 2-(cyclohexyloxy)-5-((4-methoxybenzyl)oxy) aniline (469 mg, 1.43 mmol) was reacted to give the title compound (350 mg, 52%).

¹H NMR (400 MHz, CDCl₃) δ 7.40 (d, J=2.9 Hz, 1H), 7.34-7.31 (m, 2H), 6.92-6.86 (m, 3H), 6.77 (d, J=9.4 Hz, 1H), 4.91-4.90 (m, 2H), 4.18 (tt, J=3.9, 7.7 Hz, 1H), 3.81 (s, 3H), 1.93-1.79 (m, 4H), 1.69-1.51 (m, 2H), 1.40-1.25 (m, 4H).

### Preparation 59: 4-(2-(cyclohexyloxy)-5-((4-methoxybenzyl)oxy)phenyl)-6-methyl-1-tosyl-1,6-dihydro-7H-pyrrolo[2,3-c]pyridin-7-one

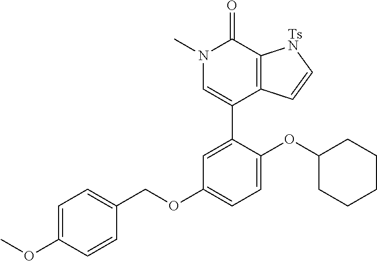 [SMILES: COc1ccc(COc2ccc(OC3CCCCC3)c(-c3cn(C)c(=O)c4c3ccn4S(=O)(=O)c3ccc(C)cc3)c2)cc1]

Following the procedure in preparation 40, 1-(cyclohexyloxy)-2-iodo-4-((4-methoxybenzyl)oxy)benzene (169 mg, 0.39 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (166 mg, 0.39 mmol) was reacted to give the title compound (107 mg, 43%).

HPLC t_{R} (Agilent, acidic, 3.5 min): 2.21 min, m/z=613.3 [M+H]⁺.

### Preparation 60: 4-(2-(cyclohexyloxy)-5-hydroxyphenyl)-6-methyl-1-tosyl-1,6-dihydro-7H-pyrrolo[2,3-c]pyridin-7-one

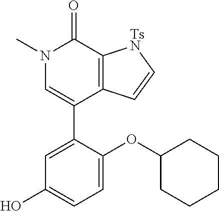 [SMILES: Cc1ccc(S(=O)(=O)n2ccc3c(-c4cc(O)ccc4OC4CCCCC4)cn(C)c(=O)c32)cc1]

Following the procedure in preparation 48, 4-(2-(cyclohexyloxy)-5-((4-methoxybenzyl)oxy)phenyl)-6-methyl-1-tosyl-1,6-dihydro-7H-pyrrolo[2,3-c]pyridin-7-one (105 mg, 0.17 mmol) was reacted to give the title compound (58 mg, 62%).

HPLC t_{R} (Agilent, acidic, 3.5 min): 1.86 min, m/z=493.3 [M+H]⁺.

### Preparation 61: 4-(2-(cyclohexyloxy)-5-hydroxyphenyl)-6-methyl-1,6-dihydro-7H-pyrrolo[2,3-c]pyridin-7-one

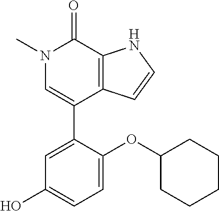 [SMILES: Cn1cc(-c2cc(O)ccc2OC2CCCCC2)c2cc[nH]c2c1=O]

Following the procedure in preparation 49, 4-(2-(cyclohexyloxy)-5-hydroxyphenyl)-6-methyl-1-tosyl-1,6-dihydro-7H-pyrrolo[2,3-c]pyridin-7-one (58 mg, 0.12 mmol) was reacted to give the title compound (12 mg, 29%).

HPLC t_{R} (Agilent, acidic, 3.5 min): 1.51 min, m/z=339.2 [M+H]⁺.

¹H NMR (400 MHz, DMSO) & 11.95 (s, 1H), 9.01 (s, 1H), 7.27-7.22 (m, 2H), 6.91 (d, J=8.7 Hz, 1H), 6.79 (d, J=3.0 Hz, 1H), 6.67 (dd, J=2.9, 8.7 Hz, 1H), 6.18-6.16 (m, 1H), 4.00-3.94 (m, 1H), 3.54 (s, 3H), 1.66-1.65 (m, 2H), 1.52-1.49 (m, 2H), 1.37 (s, 1H), 1.28-1.25 (m, 3H), 1.19-1.14 (m, 2H)

### Example 12: 4-(5-hydroxy-2-((4-methoxycyclohexyl)oxy)phenyl)-6-methyl-1,6-dihydro-7H-pyrrolo[2,3-c]pyridin-7-one

### Preparation 62: 4-methoxycyclohexan-1-ol

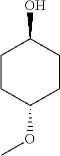

To a solution of cyclohexane-1,4-diol (4.6 g, 39.6 mmol) in DMF (15 mL) was added sodium hydride, 60% in oil (1.74 g, 43.5 mmol) at 20 °C and stirred for 30 minutes. Iodomethane (0.6 mL, 8.1 mmol) was added and the reaction was stirred at 20 °C for 16 hours. The reaction was concentrated under vacuum. The residue was dissolved in EtOAc (2×100 mL) and washed with H₂O (100 mL), the combined organic phase was washed with brine (100 mL), dried with anhydrous Na₂SO₄, filtered and concentrated in vacuum to give 4-((4-methoxybenzyl)oxy)-2-nitro-1-propoxybenzene (845 mg, 5.84 mmol, yield=15%) as a colourless oil.

¹H NMR (400 MHz, CDCl₃) δ 3.70-3.64 (m, 1H), 3.33 (s, 3H), 3.21-3.13 (m, 1H), 2.04-1.94 (m, 4H), 1.35-1.24 (m, 4H).

### Preparation 63: 4-((4-methoxybenzyl)oxy)-1-((4-methoxycyclohexyl)oxy)-2-nitrobenzene

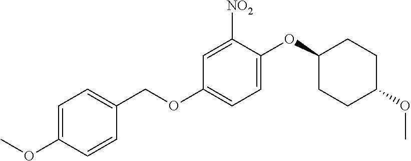

Following the procedure in preparation 44, 1-fluoro-4-((4-methoxybenzyl)oxy)-2-nitrobenzene (600 mg, 2.2 mmol) and 4-methoxycyclohexan-1-ol (845 mg, 6.5 mmol) was reacted to give the title compound (684 mg, 78%).

¹H NMR (400 MHz, CDCl₃) δ 7.44 (d, J=3.1 Hz, 1H), 7.38-7.34 (m, 2H), 7.14 (dd, J=3.1, 9.2 Hz, 1H), 7.04 (d, J=9.3 Hz, 1H), 6.96-6.93 (m, 2H), 4.99 (s, 2H), 4.43-4.36 (m, 1H), 3.85 (s, 3H), 3.40-3.33 (m, 4H), 2.07-2.01 (m, 4H), 1.72-1.63 (m, 2H), 1.57-1.47 (m, 2H).

### Preparation 64: 5-((4-methoxybenzyl)oxy)-2-((4-methoxycyclohexyl)oxy) aniline

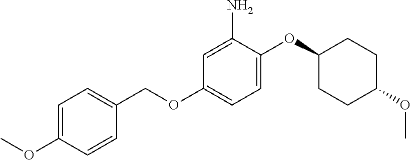

Following the procedure in preparation 44, 4-((4-methoxybenzyl)oxy)-1-((4-methoxycyclohexyl)oxy)-2-nitrobenzene (684 mg, 1.76 mmol) was reacted to give the title compound (506 mg, 76%).

HPLC t_{R} (Agilent, acidic, 3.5 min): 1.67 min, m/z=358.2 [M+H]⁺.

### Preparation 65: 2-iodo-4-((4-methoxybenzyl)oxy)-1-((4-methoxycyclohexyl)oxy)benzene

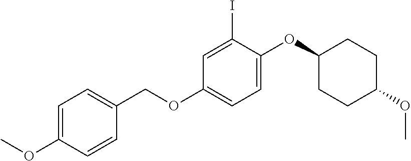

Following the procedure in preparation 45, 5-((4-methoxybenzyl)oxy)-2-((4-methoxycyclohexyl)oxy) aniline (506 mg, 1.41 mmol) was reacted to give the title compound (170 mg, 23%).

¹H NMR (400 MHz, CDCl₃) δ 7.42 (d, J=2.9 Hz, 1H), 7.35 (d, J=8.6 Hz, 2H), 6.95-6.89 (m, 3H), 6.80 (d, J=9.0 Hz, 1H), 4.93 (s, 2H), 4.30-4.24 (m, 1H), 3.84 (s, 3H), 3.37 (s, 4H), 2.12-2.01 (m, 4H), 1.71-1.44 (m, 4H).

### Preparation 66: 4-(5-((4-methoxybenzyl)oxy)-2-((4-methoxycyclohexyl)oxy)phenyl)-6-methyl-1-tosyl-1,6-dihydro-7H-pyrrolo[2,3-c]pyridin-7-one

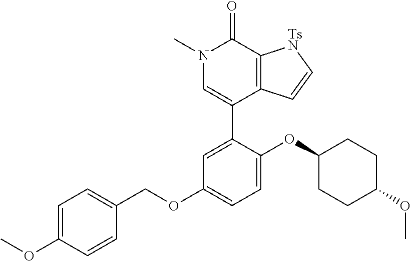

Following the procedure in preparation 40, 2-iodo-4-((4-methoxybenzyl)oxy)-1-((4-methoxycyclohexyl)oxy)benzene (169 mg, 0.36 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (155 mg, 0.36 mmol) was reacted to give the title compound (185 mg, 72%).

HPLC t_{R} (Agilent, acidic, 3.5 min): 1.97 min, m/z=643.3 [M+H]⁺.

### Preparation 67: 4-(5-hydroxy-2-((4-methoxycyclohexyl)oxy)phenyl)-6-methyl-1-tosyl-1,6-dihydro-7H-pyrrolo[2,3-c]pyridin-7-one

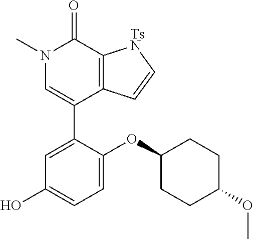 [SMILES: CO[C@H]1CC[C@H](Oc2ccc(O)cc2-c2cn(C)c(=O)c3c2ccn3S(=O)(=O)c2ccc(C)cc2)CC1]

Following the procedure in preparation 48, 4-(5-((4-methoxybenzyl)oxy)-2-((4-methoxycyclohexyl)oxy)phenyl)-6-methyl-1-tosyl-1,6-dihydro-7H-pyrrolo[2,3-c]pyridin-7-one (185 mg, 0.29 mmol) was reacted to give the title compound (97 mg, 58%).

HPLC t_{R} (Agilent, acidic, 3.5 min): 1.63 min, m/z=523.3 [M+H]⁺.

### Preparation 68: 4-(5-hydroxy-2-((4-methoxycyclohexyl)oxy)phenyl)-6-methyl-1,6-dihydro-7H-pyrrolo[2,3-c]pyridin-7-one

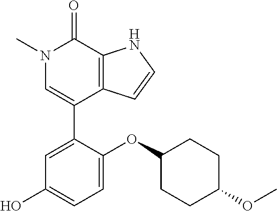

Following the procedure in preparation 49, 4-(5-hydroxy-2-((4-methoxycyclohexyl)oxy)phenyl)-6-methyl-1-tosyl-1,6-dihydro-7H-pyrrolo[2,3-c]pyridin-7-one (94 mg, 0.18 mmol) was reacted to give the title compound (21 mg, 30%).

HPLC t_{R} (Agilent, acidic, 8 min): 2.95 min, m/z=369.2 [M+H]⁺.

¹H NMR (400 MHz, DMSO) δ 11.92 (s, 1H), 8.99 (s, 1H), 7.29-7.23 (m, J=2.7 Hz, 1H), 7.21 (s, 1H), 6.97-6.88 (m, J=8.7 Hz, 1H), 6.80 (d, J=3.0 Hz, 1H), 6.68 (dd, J=8.8, 3.0 Hz, 1H), 6.16 (d, J=2.2 Hz, 1H), 4.10-3.91 (m, 1H), 3.55 (s, 3H), 3.16 (s, 3H), 3.12-3.02 (m, 1H), 1.87-1.65 (m, 4H), 1.32-1.11 (m, 4H).

### Example 13: 4-(5-benzyl-1-methyl-2-oxo-1,2-dihydropyridin-4-yl)-6-methyl-1,6-dihydro-7H-pyrrolo[2,3-c]pyridin-7-one

### Preparation 69: 5-benzyl-2-chloropyridin-4-amine

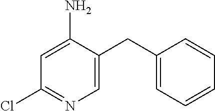 [SMILES: Nc1cc(Cl)ncc1Cc1ccccc1]

A mixture of 5-bromo-2-chloropyridin-4-amine (4.60 g, 22.17 mmol), 2-benzyl-4,4,5,5-tetramethyl-1,3,2-dioxaborolane (6.00 g, 27.51 mmol), K₃PO₄ (13.8 g, 65.0 mmol, 2.93 eq) and cataCXium A Pd-G3 (500 mg, 687 μmol, 0.031 eq) in H₂O (8 mL) and dioxane (40 mL) was stirred at 75 °C for 12 hours under N₂. The mixture was poured into water (200 mL) and extracted with EtOAc (50 mL×3). The combined organic layer was washed with brine (50 mL), dried over Na₂SO₄, filtered and concentrated. The residue was purified by column chromatography (SiO₂, Petroleum ether:Ethyl acetate=10:1-5:1-3:1) (Petroleum ether:Ethyl acetate=3:1, Rf=0.5) to give the title compound (3.60 g, 16.5 mmol, 74.2% yield) as yellow solid.

¹H NMR (400 MHz, CDCl₃) δ 8.09 (s, 1H), 7.34-7.32 (m, 2H), 7.31-7.30 (m, 1H), 7.25-717 (m, 2H), 6.55 (s, 1H), 4.16-4.10 (m, 2H), 3.85 (s, 2H)

### Preparation 70: 5-benzyl-4-bromo-2-chloropyridine

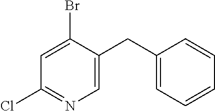 [SMILES: Clc1cc(Br)c(Cc2ccccc2)cn1]

A mixture of tert-butyl nitrite (2.70 g, 26.2 mmol) and CuBr (4.81 g, 33.5 mmol) was stirred in MeCN (10 mL) at 70 °C for 10 minutes. A solution of 5-benzyl-2-chloropyridin-4-amine (1.80 g, 8.23 mmol) in MeCN (10 mL) was added drop-wise to the reaction mixture at 70 °C, and the mixture was stirred at 70 °C for 1 hour. The mixture was poured into water (80 mL) and extracted with ethyl acetate (100 mL). The combined organic layer was washed with brine (50 mL), dried over Na₂SO₄, filtered and concentrated to give the title compound (2.00 g, 7.08 mmol, 86.0% yield) as green oil.

¹H NMR (400 MHz, CDCl₃) δ 7.37-7.29 (m, 2H), 7.29-7.23 (m, 2H), 7.17 (d, J=7.0 Hz, 3H), 4.29 (s, 2H).

### Preparation 71: 5-benzyl-4-bromo-1-methylpyridin-2 (1H)-one

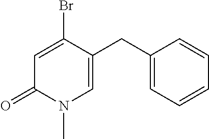 [SMILES: Cn1cc(Cc2ccccc2)c(Br)cc1=O]

5-benzyl-4-bromo-2-chloropyridine (2.00 g, 7.08 mmol) was dissolved in CHCl₃ (10 mL), Me₂SO₄ (5.35 mL, 56.4 mmol) was added and the solution heated at 70 °C for 12 hrs. Upon cooling, a mixture of TEA (15.0 g, 148 mmol), CH₃CO₂H (13.7 mL, 240 mmol), and EtOH (13.7 mL, 235 mmol) was added and the reaction heated at 70 °C for a further 2 hrs. The reaction mixture diluted with H₂O (50 mL) and then extracted with ethyl acetate 200 mL. The combined organic layers were washed with brine (50 mL), dried over Na₂SO₄, filtered and concentrated under reduced pressure to give an oil. The residue was purified by prep-HPLC (HCl condition; column: Phenomenex luna C18 250×50 mm×10 μm, to give 5-benzyl-4-bromo-1-methylpyridin-2 (1H)-one (859 mg, 3.09 mmol, 43.6% yield) as a yellow solid.

¹H NMR (400 MHz, DMSO-d6) δ 7.80 (s, 1H), 7.33-7.26 (m, 2H), 7.23-7.17 (m, 3H), 6.77 (s, 1H), 3.81 (s, 2H), 3.41 (s, 3H).

### Preparation 72: 4-(5-benzyl-1-methyl-2-oxo-1,2-dihydropyridin-4-yl)-6-methyl-1,6-dihydro-7H-pyrrolo[2,3-c]pyridin-7-one

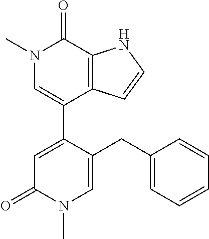 [SMILES: Cn1cc(Cc2ccccc2)c(-c2cn(C)c(=O)c3[nH]ccc23)cc1=O]

Following the procedure in preparation 10, 5-benzyl-4-bromo-1-methylpyridin-2 (1H)-one (71 mg, 0.26 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (100 mg, 0.23 mmol) was reacted to give the title compound (34 mg, 39%).

HPLC t_{R} (Agilent, acidic, 3.5 min): 1.26 min, m/z=346.2 [M+H]⁺.

¹H NMR (500 MHz, CDCl₃) δ 10.61 (bs, 1H), 7.21 (t, J=2.4 Hz, 1H), 7.13-7.07 (m, 3H), 7.01 (s, 1H), 6.77 (d, J=6.6 Hz, 2H), 6.50 (s, 1H), 6.35 (s, 1H), 6.14 (t, J=2.4 Hz, 1H), 5.22 (s, 2H), 3.53 (s, 3H), 3.42 (s, 3H).

### Example 14: 4-(1-benzyl-1H-pyrazol-5-yl)-6-methyl-1,6-dihydro-7H-pyrrolo[2,3-c]pyridin-7-one

### Preparation 73: 4-(5-benzyl-1-methyl-2-oxo-1,2-dihydropyridin-4-yl)-6-methyl-1,6-dihydro-7H-pyrrolo[2,3-c]pyridin-7-one

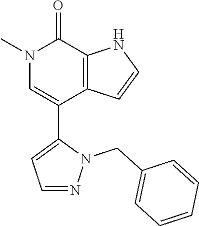 [SMILES: Cn1cc(-c2ccnn2Cc2ccccc2)c2cc[nH]c2c1=O]

Following the procedure in preparation 10, 1-benzyl-5-bromo-1H-pyrazole (61 mg, 0.26 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (100 mg, 0.23 mmol) was reacted to give the title compound (17 mg, 23%).

HPLC t_{R} (Agilent, acidic, 3.5 min): 1.41 min, m/z=305.2 [M+H]⁺.

¹H NMR (500 MHz, CDCl₃) δ 9.61 (bs, 1H), 7.68 (d, J=1.8 Hz, 1H), 7.28-7.25 (m, 4H), 7.05-7.02 (m, 2H), 6.70 (s, 1H), 6.41 (d, J=1.8 Hz, 1H), 6.27 (t, J=2.6 Hz, 1H), 5.34-5.33 (m, 2H), 3.55 (s, 3H).

### Example 15: 4-(1-benzyl-1H-imidazol-2-yl)-6-methyl-1,6-dihydro-7H-pyrrolo[2,3-c]pyridin-7-one

### Preparation 74: 1-benzyl-2-bromo-1H-imidazole

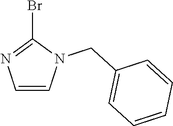 [SMILES: Brc1nccn1Cc1ccccc1]

To a solution of 2-bromo-1H-imidazole (1.0 g, 7.1 mmol) in TMF (160 mL) was added sodium hydride, 60% in oil (286 mg, 7.1 mmol) at 20 °C and stirred for 10 minutes at 70 °C (bromomethyl)benzene (0.85 mL, 8.1 mmol) was added and the reaction was stirred at 70 °C for 1 hour. The reaction mixture was added to EtOAc (100 mL) and washed with H₂O (100 mL), the organic phase was washed with brine (100 mL), dried with anhydrous Na₂SO₄, filtered and concentrated in vacuum to give 1-benzyl-2-bromo-1H-imidazole (920 mg, 54%).

HPLC t_{R} (Agilent, acidic, 3.5 min): 1.29 min, m/z=238.1 [M+H]⁺.

### Preparation 75: 4-(1-benzyl-1H-imidazol-2-yl)-6-methyl-1,6-dihydro-7H-pyrrolo[2,3-c]pyridin-7-one

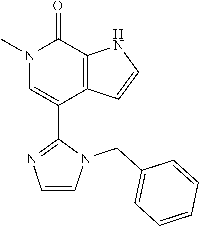 [SMILES: Cn1cc(-c2nccn2Cc2ccccc2)c2cc[nH]c2c1=O]

Following the procedure in preparation 10, 1-benzyl-2-bromo-1H-imidazole (61 mg, 0.26 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (100 mg, 0.23 mmol) was reacted to give the title compound (18 mg, 24%).

HPLC t_{R} (Agilent, acidic, 3.5 min): 0.97 min, m/z=305.2 [M+H]⁺.

¹H NMR (500 MHz, CDCl₃) δ 10.09 (bs, 1H), 7.36-7.33 (m, 2H), 7.31 (t, J=2.9 Hz, 2H), 7.27 (d, J=1.1 Hz, 1H), 7.12 (s, 1H), 7.06 (t, J=1.2 Hz, 1H), 7.04 (d, J=7.4 Hz, 2H), 6.41 (t, J=2.5 Hz, 1H), 5.18 (s, 2H), 3.62 (s, 3H).

### Example 16: 4-(1-benzyl-1H-1,2,4-triazol-5-yl)-6-methyl-1,6-dihydro-7H-pyrrolo[2,3-c]pyridin-7-one

### Preparation 76: 4-(1-benzyl-1H-1,2,4-triazol-5-yl)-6-methyl-1,6-dihydro-7H-pyrrolo[2,3-c]pyridin-7-one

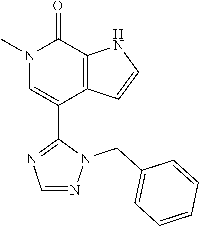 [SMILES: Cn1cc(-c2ncnn2Cc2ccccc2)c2cc[nH]c2c1=O]

Following the procedure in preparation 10, 1-benzyl-2-bromo-1H-imidazole (61 mg, 0.26 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (100 mg, 0.23 mmol) was reacted to give the title compound (18 mg, 24%).

HPLC t_{R} (Agilent, acidic, 3.5 min): 0.97 min, m/z=305.2 [M+H]⁺.

¹H NMR (500 MHz, DMSO-d₆) δ 12.22 (bs, 1H), 8.12 (s, 1H), 7.48 (s, 1H), 7.36-7.26 (m, 4H), 7.09 (d, J=7.0 Hz, 2H), 6.35 (d, J=2.4 Hz, 1H), 5.50 (s, 2H), 3.30 (s, 3H).

### Example 17: 4-(4-benzylthiazol-5-yl)-6-methyl-1,6-dihydro-7H-pyrrolo[2,3-c]pyridin-7-one

### Preparation 77: 4-benzyl-5-bromothiazole

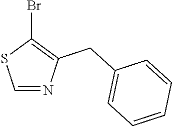 [SMILES: Brc1scnc1Cc1ccccc1]

To the solution of 4-benzyl-5-bromothiazol-2-amine (16.2 g, 60.2 mmol) in DMF (160 mL) which was heated to 55 °C was added dropwise solution of tert-butyl nitrite (9.3 g, 90.2 mmol) in DMF (50 mL). The reaction mixture was stirred for 1 h at 70 °C. After that it was cooled to RT water (250 mL) was added, and then water layer was extracted with EtOAc (3×100 mL). Combined organics were dried over Na₂SO₄ and concentrated under reduced pressure. The residue was purified by column chromatography (eluent Hexane:EtOAc 14:1) to give 4-benzyl-5-bromothiazole (1.1 g, 7.2% yield)

¹H NMR (400 MHz, CDCl₃) δ 8.68 (s, 1H), 7.25-7.20 (m, 4H), 7.17-7.12 (m, 1H), 4.09 (s, 2H).

### Preparation 78: 4-(4-benzylthiazol-5-yl)-6-methyl-1,6-dihydro-7H-pyrrolo[2,3-c]pyridin-7-one

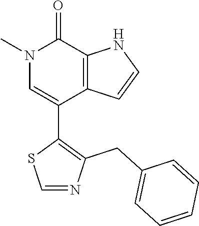 [SMILES: Cn1cc(-c2scnc2Cc2ccccc2)c2cc[nH]c2c1=O]

Following the procedure in preparation 10, 4-benzyl-5-bromothiazole (65 mg, 0.26 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (100 mg, 0.23 mmol) was reacted to give the title compound (5 mg, 6%). HPLC t_{R} (Agilent, acidic, 3.5 min): 1.43 min, m/z=322.2 [M+H]⁺.

¹H NMR (500 MHz, DMSO-d₆) δ 11.18 (bs, 1H), 8.86 (s, 1H), 7.34 (t, J=2.6 Hz, 1H), 7.30-7.28 (m, 2H), 7.23-7.18 (m, 3H), 6.89 (s, 1H), 6.34 (t, J=2.5 Hz, 1H), 4.18-4.17 (m, 2H), 3.66-3.65 (m, 3H).

### Example 18: 1-methyl-5-(4-phenoxythiazol-5-yl)pyridin-2 (1H)-one

### Preparation 79: 4-(4-benzylthiazol-5-yl)-6-methyl-1,6-dihydro-7H-pyrrolo[2,3-c]pyridin-7-one

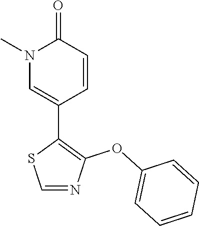 [SMILES: Cn1cc(-c2scnc2Oc2ccccc2)ccc1=O]

Following the procedure in preparation 40, 5-bromo-4-phenoxythiazole (89 mg, 0.35 mmol) was reacted to give the title compound (48 mg, 48%).

HPLC t_{R} (Agilent, acidic, 3.5 min): 1.34 min, m/z=285.0 [M+H]⁺.

¹H NMR (500 MHz, DMSO-d₆) δ 8.93 (s, 1H), 8.06 (s, 1H), 7.70-7.66 (m, 1H), 7.37 (t, J=7.3 Hz, 2H), 7.12 (t, J=7.4 Hz, 1H), 7.04-7.00 (m, 2H), 6.48-6.45 (m, 1H), 3.47 (s, 3H).

### Example 19: 4-(4-(2-hydroxyphenoxy) thiazol-5-yl)-6-methyl-1,6-dihydro-7H-pyrrolo[2,3-c]pyridin-7-one

### Preparation 80: 4-(2-methoxyphenoxy) thiazole

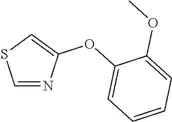

Following the procedure in preparation 22, 2-methoxyphenol (1.1 g, 8.65 mmol) was reacted to give the title compound (307 mg, 17%).

HPLC t_{R} (Agilent, acidic, 3.5 min): 1.47 min, m/z=208.0 [M+H]⁺.

### Preparation 81: 5-bromo-4-(2-methoxyphenoxy) thiazole

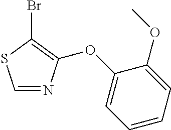

Following the procedure in preparation 23, 4-(2-methoxyphenoxy) thiazole (155 mg, 0.75 mmol) was reacted to give the title compound (170 mg, 79%).

HPLC t_{R} (Agilent, acidic, 3.5 min): 1.65 min, m/z=287.2 [M+H]⁺.

### Preparation 82: 4-(4-(2-methoxyphenoxy) thiazol-5-yl)-6-methyl-1,6-dihydro-7H-pyrrolo[2,3-c]pyridin-7-one

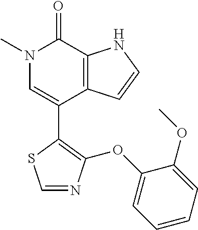 [SMILES: COc1ccccc1Oc1ncsc1-c1cn(C)c(=O)c2[nH]ccc12]

Following the procedure in preparation 10, 5-bromo-4-(2-methoxyphenoxy) thiazole (169 mg, 0.59 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (230 mg, 0.54 mmol) was reacted to give the title compound (43 mg, 23%).

HPLC t_{R} (Agilent, acidic, 3.5 min): 1.46 min, m/z=354.2 [M+H]⁺.

### Preparation 83: 4-(4-(2-hydroxyphenoxy) thiazol-5-yl)-6-methyl-1,6-dihydro-7H-pyrrolo[2,3-c]pyridin-7-one

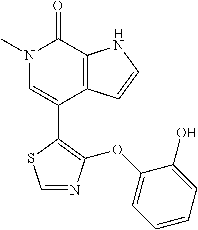 [SMILES: Cn1cc(-c2scnc2Oc2ccccc2O)c2cc[nH]c2c1=O]

Following the procedure in preparation 11, 4-(4-(2-methoxyphenoxy) thiazol-5-yl)-6-methyl-1,6-dihydro-7H-pyrrolo[2,3-c]pyridin-7-one (43 mg, 0.12 mmol) was reacted to give the title compound (9 mg, 20%).

HPLC t_{R} (Agilent, acidic, 3.5 min): 1.36 min, m/z=340.0 [M+H]⁺.

¹H NMR (500 MHz, DMSO-d₆) δ 12.21 (bs, 1H), 9.48 (bs, 1H), 8.88 (s, 1H), 7.69 (s, 1H), 7.36 (t, J=2.6 Hz, 1H), 6.98-6.89 (m, 3H), 6.75-6.71 (m, 1H), 6.56 (t, J=2.1 Hz, 1H), 3.55 (s, 3H).

### Example 20: 4-(4-(4-hydroxyphenoxy) thiazol-5-yl)-6-methyl-1,6-dihydro-7H-pyrrolo[2,3-c]pyridin-7-one

### Preparation 84: 4-(4-methoxyphenoxy) thiazole

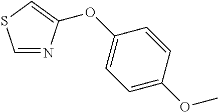

Following the procedure in preparation 22, 4-methoxyphenol (1.1 g, 8.65 mmol) was reacted to give the title compound (332 mg, 19%).

HPLC t_{R} (Agilent, acidic, 3.5 min): 1.46 min, m/z=208.0 [M+H]⁺.

### Preparation 85: 5-bromo-4-phenoxythiazole

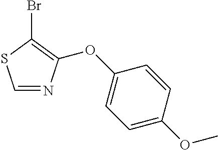

Following the procedure in preparation 23, 4-(2-methoxyphenoxy) thiazole (280 mg, 1.35 mmol) was reacted to give the title compound (195 mg, 50%).

HPLC t_{R} (Agilent, acidic, 3.5 min): 1.70 min, m/z=287.2 [M+H]⁺.

### Preparation 86: 4-(4-(4-methoxyphenoxy) thiazol-5-yl)-6-methyl-1,6-dihydro-7H-pyrrolo[2,3-c]pyridin-7-one

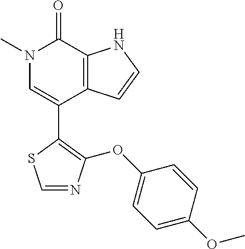

Following the procedure in preparation 10, 5-bromo-4-phenoxythiazole (162 mg, 0.57 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (220 mg, 0.51 mmol) was reacted to give the title compound (40 mg, 22%).

HPLC t_{R} (Agilent, acidic, 3.5 min): 1.43 min, m/z=354.0 [M+H]⁺.

### Preparation 87: 4-(4-(4-hydroxyphenoxy) thiazol-5-yl)-6-methyl-1,6-dihydro-7H-pyrrolo[2,3-c]pyridin-7-one

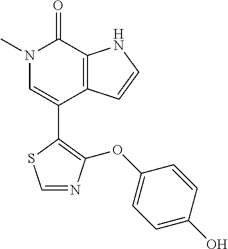 [SMILES: Cn1cc(-c2scnc2Oc2ccc(O)cc2)c2cc[nH]c2c1=O]

Following the procedure in preparation 11, 4-(4-(2-methoxyphenoxy) thiazol-5-yl)-6-methyl-1,6-dihydro-7H-pyrrolo[2,3-c]pyridin-7-one (40 mg, 0.11 mmol) was reacted to give the title compound (15 mg, 35%).

HPLC t_{R} (Agilent, acidic, 3.5 min): 1.30 min, m/z=340.0 [M+H]⁺.

¹H NMR (500 MHz, DMSO-d₆) δ 12.19 (bs, 1H), 9.18 (bs, 1H), 8.95 (s, 1H), 7.53 (s, 1H), 7.35 (t, J=2.7 Hz, 1H), 6.86-6.83 (m, 2H), 6.71-6.69 (m, 2H), 6.44 (t, J=2.4 Hz, 1H), 3.54 (s, 3H).

### Example 21: 5-(4-(2-hydroxyphenoxy) thiazol-5-yl)-1-methylpyridin-2 (1H)-one

### Preparation 88: 5-(4-(2-methoxyphenoxy) thiazol-5-yl)-1-methylpyridin-2 (1H)-one

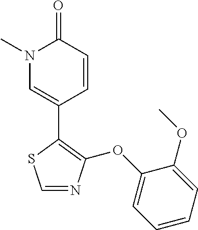 [SMILES: COc1ccccc1Oc1ncsc1-c1ccc(=O)n(C)c1]

Following the procedure in preparation 40, 5-bromo-4-(2-methoxyphenoxy) thiazole (167 mg, 0.58 mmol) was reacted to give the title compound (110 mg, 66%).

HPLC t_{R} (Agilent, acidic, 3.5 min): 1.35 min, m/z=315.0 [M+H]⁺.

### Preparation 89: 5-(4-(2-hydroxyphenoxy) thiazol-5-yl)-1-methylpyridin-2 (1H)-one

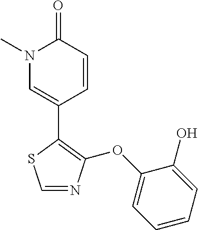 [SMILES: Cn1cc(-c2scnc2Oc2ccccc2O)ccc1=O]

Following the procedure in preparation 11, 4-(4-(2-methoxyphenoxy) thiazol-5-yl)-6-methyl-1,6-dihydro-7H-pyrrolo[2,3-c]pyridin-7-one (110 mg, 0.35 mmol) was reacted to give the title compound (53 mg, 46%).

HPLC t_{R} (Agilent, acidic, 3.5 min): 1.26 min, m/z=301.0 [M+H]⁺.

¹H NMR (500 MHz, DMSO-d₆) δ 9.54 (s, 1H), 8.79 (s, 1H), 8.12 (s, 1H), 7.83-7.81 (d, J=9.7 Hz, 1H), 6.99-6.90 (m, 3H), 6.75 (t, J=7.6 Hz, 1H), 6.48 (d, J=10.4 Hz, 1H), 3.48 (s, 3H).

### Example 22: 4-(5-(2-hydroxyphenoxy)-1-methyl-2-oxo-1,2-dihydropyridin-4-yl)-6-methyl-1,6-dihydro-7H-pyrrolo[2,3-c]pyridin-7-one

### Preparation 90: 2-chloro-5-(2-methoxyphenoxy)-4-nitropyridine 1-oxide

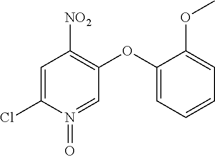 [SMILES: COc1ccccc1Oc1c[n+]([O-])c(Cl)cc1[N+](=O)[O-]]

Following the procedure in preparation 35, 2-methoxyphenol (15.5 g, 125 mmol) was reacted to give the title compound (23.0 g, 75%).

HPLC t_{R} (Shimadzu, acidic, 1.5 min): 0.92 min, m/z=297.1 [M+H]⁺.

### Preparation 91: 2,4-dibromo-5-(2-methoxyphenoxy)pyridine 1-oxide

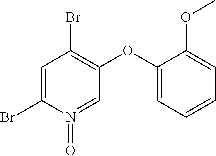 [SMILES: COc1ccccc1Oc1c[n+]([O-])c(Br)cc1Br]

Following the procedure in preparation 26, 2-chloro-5-(2-methoxyphenoxy)-4-nitropyridine 1-oxide (6.0 g, 20.2 mmol) was reacted to give the title compound (7.0 g, 92%).

HPLC t_{R} (Shimadzu, acidic, 1.5 min): 0.85 min, m/z=376.0 [M+H]⁺.

### Preparation 92: 2,4-dibromo-5-(2-methoxyphenoxy)pyridine

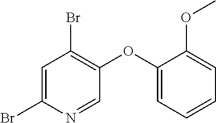 [SMILES: COc1ccccc1Oc1cnc(Br)cc1Br]

Following the procedure in preparation 27, 2,4-dibromo-5-(2-methoxyphenoxy)pyridine 1-oxide (7.0 g, 18.6 mmol) was reacted to give the title compound (6.7 g, 100%).

HPLC t_{R} (Shimadzu, acidic, 1.5 min): 0.91 min, m/z=359.9 [M+H]⁺.

### Preparation 93: 4-bromo-5-(2-methoxyphenoxy)pyridin-2 (1H)-one

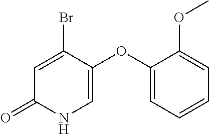 [SMILES: COc1ccccc1Oc1c[nH]c(=O)cc1Br]

Following the procedure in preparation 38, 2,4-dibromo-5-(2-methoxyphenoxy)pyridine (6.7 g, 18.6 mmol) was reacted to give the title compound (4.3 g, 77%).

HPLC t_{R} (Shimadzu, acidic, 1.5 min): 0.81 min, m/z=298.0 [M+H]⁺.

### Preparation 94: 4-bromo-5-(2-methoxyphenoxy)-1-methylpyridin-2 (1H)-one

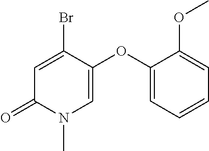 [SMILES: COc1ccccc1Oc1cn(C)c(=O)cc1Br]

Following the procedure in preparation 39, 4-bromo-5-(2-methoxyphenoxy)pyridin-2 (1H)-one (4.2 g, 14.3 mmol) was reacted to give the title compound (50 mg, 1%).

¹H NMR (400 MHz, DMSO-d₆) δ 7.78 (s, 1H), 7.13-7.02 (m, 2H), 6.90-6.84 (m, 2H), 6.80-6.76 (m, 1H), 3.82 (s, 3H), 3.37 (s, 3H) Preparation 95: 4-(5-(2-methoxyphenoxy)-1-methyl-2-oxo-1,2-dihydropyridin-4-yl)-6-methyl-1,6-dihydro-7H-pyrrolo[2,3-c]pyridin-7-one

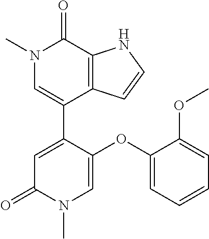 [SMILES: COc1ccccc1Oc1cn(C)c(=O)cc1-c1cn(C)c(=O)c2[nH]ccc12]

Following the procedure in preparation 10, 4-bromo-5-(2-methoxyphenoxy)-1-methylpyridin-2 (1H)-one (36 mg, 0.11 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (50 mg, 0.11 mmol) was reacted to give the title compound (8 mg, 18%).

HPLC t_{R} (Agilent, acidic, 3.5 min): 1.25 min, m/z=378.1 [M+H]⁺.

### Preparation 96: 4-(5-(2-hydroxyphenoxy)-1-methyl-2-oxo-1,2-dihydropyridin-4-yl)-6-methyl-1,6-dihydro-7H-pyrrolo[2,3-c]pyridin-7-one

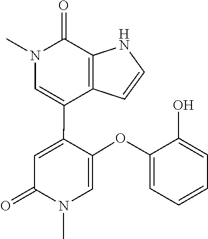 [SMILES: Cn1cc(Oc2ccccc2O)c(-c2cn(C)c(=O)c3[nH]ccc23)cc1=O]

Following the procedure in preparation 11, 4-(5-(2-methoxyphenoxy)-1-methyl-2-oxo-1,2-dihydropyridin-4-yl)-6-methyl-1,6-dihydro-7H-pyrrolo[2,3-c]pyridin-7-one (24 mg, 0.06 mmol) was reacted to give the title compound (13 mg, 50%).

HPLC t_{R} (Agilent, acidic, 3.5 min): 1.17 min, m/z=364.0 [M+H]⁺.

¹H NMR (500 MHz, CDCl₃) δ 9.56 (bs, 1H), 7.06 (s, 1H), 7.00 (s, 1H), 6.81-6.81 (m, 1H), 6.69-6.62 (m, 2H), 6.56 (s, 1H), 6.50-6.47 (m, 2H), 6.26 (t, J=2.5 Hz, 1H), 5.55 (bs, 1H), 3.36 (s, 3H), 3.32 (s, 3H).

### Example 23: 4-(5-(3-methoxyphenoxy)-1-methyl-2-oxo-1,2-dihydropyridin-4-yl)-6-methyl-1,6-dihydro-7H-pyrrolo[2,3-c]pyridin-7-one

### Preparation 97: 2-chloro-5-(3-methoxyphenoxy)-4-nitropyridine 1-oxide

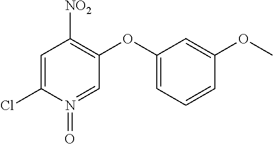 [SMILES: COc1cccc(Oc2c[n+]([O-])c(Cl)cc2[N+](=O)[O-])c1]

Following the procedure in preparation 35, 3-methoxyphenol (15.5 g, 125 mmol) was reacted to give the title compound (24.0 g, 78%).

HPLC t_{R} (Shimadzu, acidic, 1.5 min): 0.94 min, m/z=297.1 [M+H]⁺.

### Preparation 98: 2,4-dibromo-5-(3-methoxyphenoxy)pyridine 1-oxide

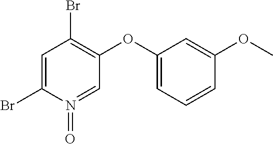 [SMILES: COc1cccc(Oc2c[n+]([O-])c(Br)cc2Br)c1]

Following the procedure in preparation 26, 2-chloro-5-(3-methoxyphenoxy)-4-nitropyridine 1-oxide (6.5 g, 21.9 mmol) was reacted to give the title compound (7.0 g, 85%).

HPLC t_{R} (Shimadzu, acidic, 1.5 min): 0.83 min, m/z=376.0 [M+H]⁺.

### Preparation 99: 2,4-dibromo-5-(3-methoxyphenoxy)pyridine

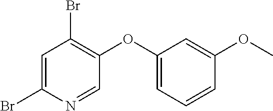 [SMILES: COc1cccc(Oc2cnc(Br)cc2Br)c1]

Following the procedure in preparation 27, 2,4-dibromo-5-(3-methoxyphenoxy)pyridine 1-oxide (15 g, 40.0 mmol) was reacted to give the title compound (2.0 g, 14%).

HPLC t_{R} (Shimadzu, acidic, 1.5 min): 1.00 min, m/z=359.9 [M+H]⁺.

### Preparation 100: 4-bromo-5-(3-methoxyphenoxy)pyridin-2 (1H)-one

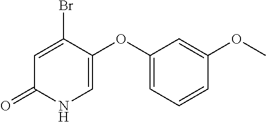 [SMILES: COc1cccc(Oc2c[nH]c(=O)cc2Br)c1]

Following the procedure in preparation 38, 2,4-dibromo-5-(3-methoxyphenoxy)pyridine (1.1 g, 3.06 mmol) was reacted to give the title compound (900 mg, 90%).

HPLC t_{R} (Agilent, acidic, 1.5 min): 0.82 min, m/z=297.1 [M+H]⁺.

### Preparation 101: 4-bromo-5-(3-methoxyphenoxy)-1-methylpyridin-2 (1H)-one

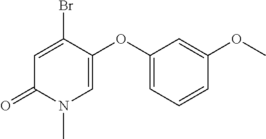 [SMILES: COc1cccc(Oc2cn(C)c(=O)cc2Br)c1]

Following the procedure in preparation 39, 4-bromo-5-(3-methoxyphenoxy)pyridin-2 (1H)-one (450 mg, 3.56 mmol) was reacted to give the title compound (0.25 g, 53%).

HPLC t_{R} (Shimadzu, acidic, 1.5 min): 0.66 min, m/z=309.8 [M+H]⁺.

### Preparation 102: 4-(5-(3-methoxyphenoxy)-1-methyl-2-oxo-1,2-dihydropyridin-4-yl)-6-methyl-1,6-dihydro-7H-pyrrolo[2,3-c]pyridin-7-one

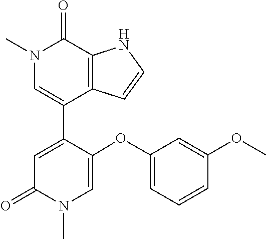 [SMILES: COc1cccc(Oc2cn(C)c(=O)cc2-c2cn(C)c(=O)c3[nH]ccc23)c1]

Following the procedure in preparation 10, 4-bromo-5-(3-methoxyphenoxy)-1-methylpyridin-2 (1H)-one (80 mg, 0.26 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (110 mg, 0.26 mmol) was reacted to give the title compound (42 mg, 40%).

HPLC t_{R} (Agilent, acidic, 3.5 min): 1.25 min, m/z=378.1 [M+H]⁺.

¹H NMR (500 MHz, CDCl₃) δ 9.63 (bs, 1H), 7.28-7.25 (m, 2H), 7.17-7.16 (m, 1H), 7.08 (t, J=8.2 Hz, 1H), 6.86-6.86 (m, 1H), 6.55 (t, J=2.9 Hz, 1H), 6.51 (dd, J=2.6, 8.4 Hz, 1H), 6.36 (dd, J=2.1, 8.3 Hz, 1H), 6.32 (t, J=2.3 Hz, 1H), 3.70-3.69 (m, 3H), 3.61 (s, 3H), 3.58 (s, 3H).

### Example 24: 4-(5-(3-hydroxyphenoxy)-1-methyl-2-oxo-1,2-dihydropyridin-4-yl)-6-methyl-1,6-dihydro-7H-pyrrolo[2,3-c]pyridin-7-one

### Preparation 103: 4-(5-(3-hydroxyphenoxy)-1-methyl-2-oxo-1,2-dihydropyridin-4-yl)-6-methyl-1,6-dihydro-7H-pyrrolo[2,3-c]pyridin-7-one

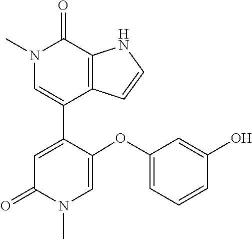 [SMILES: Cn1cc(Oc2cccc(O)c2)c(-c2cn(C)c(=O)c3[nH]ccc23)cc1=O]

Following the procedure in preparation 11, 4-(5-(3-methoxyphenoxy)-1-methyl-2-oxo-1,2-dihydropyridin-4-yl)-6-methyl-1,6-dihydro-7H-pyrrolo[2,3-c]pyridin-7-one (37 mg, 0.10 mmol) was reacted to give the title compound (22 mg, 58%).

HPLC t_{R} (Agilent, acidic, 3.5 min): 1.13 min, m/z=364.0 [M+H]⁺.

¹H NMR (500 MHz, DMSO) & 12.05 (bs, 1H), 9.37 (s, 1H), 7.85 (s, 1H), 7.38 (s, 1H), 7.31 (t, J=2.7 Hz, 1H), 6.94 (t, J=8.0 Hz, 1H), 6.55-6.54 (m, 1H), 6.34 (t, J=2.3 Hz, 1H), 6.30 (dd, J=1.9, 8.0 Hz, 1H), 6.20 (dd, J=2.2, 8.1 Hz, 1H), 6.17 (d, J=2.4 Hz, 1H), 3.48 (s, 6H).

### Example 25: 4-(5-(4-methoxyphenoxy)-1-methyl-2-oxo-1,2-dihydropyridin-4-yl)-6-methyl-1,6-dihydro-7H-pyrrolo[2,3-c]pyridin-7-one

### Preparation 104: 2-chloro-5-(4-methoxyphenoxy)-4-nitropyridine 1-oxide

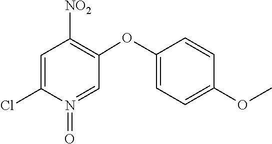 [SMILES: COc1ccc(Oc2c[n+]([O-])c(Cl)cc2[N+](=O)[O-])cc1]

Following the procedure in preparation 35, 4-methoxyphenol (4.6 g, 37.4 mmol) was reacted to give the title compound (6.0 g, 65%).

¹H NMR (400 MHz, DMSO-d₆) δ 8.69 (s, 1H), 8.11 (s, 1H), 7.21 (d, J=9.2 Hz, 2H), 7.01 (d, J=9.2 Hz, 2H), 3.77 (s, 3H)

### Preparation 105: 2,4-dibromo-5-(4-methoxyphenoxy)pyridine 1-oxide

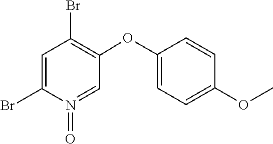 [SMILES: COc1ccc(Oc2c[n+]([O-])c(Br)cc2Br)cc1]

Following the procedure in preparation 26, 2-chloro-5-(4-methoxyphenoxy)-4-nitropyridine 1-oxide (6.0 g, 20.2 mmol) was reacted to give the title compound (7.60 g, 98%).

HPLC t_{R} (Shimadzu, acidic, 1.5 min): 0.85 min, m/z=376.0 [M+H]⁺.

### Preparation 106: 2,4-dibromo-5-(4-methoxyphenoxy)pyridine

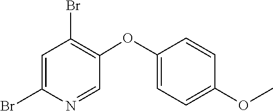 [SMILES: COc1ccc(Oc2cnc(Br)cc2Br)cc1]

Following the procedure in preparation 27, 2,4-dibromo-5-(4-methoxyphenoxy)pyridine 1-oxide (7.6 g, 20.3 mmol) was reacted to give the title compound (7.3 g, 99%).

HPLC t_{R} (Shimadzu, acidic, 1.5 min): 1.02 min, m/z=359.9 [M+H]⁺.

### Preparation 107: 4-bromo-5-(4-methoxyphenoxy)pyridin-2 (1H)-one

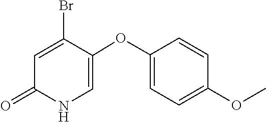 [SMILES: COc1ccc(Oc2c[nH]c(=O)cc2Br)cc1]

Following the procedure in preparation 38, 2,4-dibromo-5-(4-methoxyphenoxy)pyridine 7.3 g, 20.3 mmol) was reacted to give the title compound (4.0 g, 59%).

¹H NMR (400 MHz, DMSO-d₆) δ 7.49 (s, 1H), 6.92-6.88 (m, 4H), 6.85 (s, 1H), 3.71 (s, 3H)

### Preparation 108: 4-bromo-5-(4-methoxyphenoxy)-1-methylpyridin-2 (1H)-one

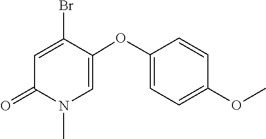 [SMILES: COc1ccc(Oc2cn(C)c(=O)cc2Br)cc1]

Following the procedure in preparation 39, 4-bromo-5-(4-methoxyphenoxy)pyridin-2 (1H)-one (4.0 g, 13.5 mmol) was reacted to give the title compound (0.7 g, 16%).

¹H NMR (400 MHz, DMSO-d₆) δ 7.90 (s, 1H), 6.92-6.87 (m, 5H), 3.71 (s, 3H), 3.39 (s, 3H)

### Preparation 109: 4-(5-(4-methoxyphenoxy)-1-methyl-2-oxo-1,2-dihydropyridin-4-yl)-6-methyl-1,6-dihydro-7H-pyrrolo[2,3-c]pyridin-7-one

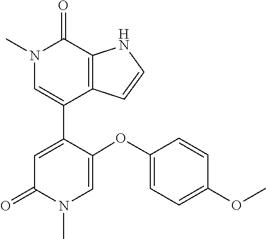 [SMILES: COc1ccc(Oc2cn(C)c(=O)cc2-c2cn(C)c(=O)c3[nH]ccc23)cc1]

Following the procedure in preparation 10, 4-bromo-5-(4-methoxyphenoxy)-1-methylpyridin-2 (1H)-one (80 mg, 0.26 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (110 mg, 0.26 mmol) was reacted to give the title compound (50 mg, 47%).

HPLC t_{R} (Agilent, acidic, 3.5 min): 1.23 min, m/z=378.1 [M+H]⁺.

¹H NMR (500 MHz, CDCl₃) δ 10.94 (bs, 1H), 7.22 (t, J=2.7 Hz, 1H), 7.09 (s, 1H), 7.04 (s, 1H), 6.76 (s, 1H), 6.64 (s, 4H), 6.43 (t, J=2.3 Hz, 1H), 3.64 (s, 3H), 3.53 (s, 3H), 3.49 (s, 3H).

### Example 26: 4-(5-(4-hydroxyphenoxy)-1-methyl-2-oxo-1,2-dihydropyridin-4-yl)-6-methyl-1,6-dihydro-7H-pyrrolo[2,3-c]pyridin-7-one

### Preparation 110: 4-(5-(4-hydroxyphenoxy)-1-methyl-2-oxo-1,2-dihydropyridin-4-yl)-6-methyl-1,6-dihydro-7H-pyrrolo[2,3-c]pyridin-7-one

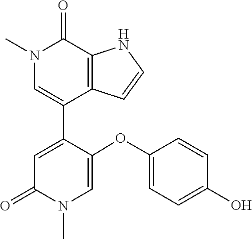 [SMILES: Cn1cc(Oc2ccc(O)cc2)c(-c2cn(C)c(=O)c3[nH]ccc23)cc1=O]

Following the procedure in preparation 11, 4-(5-(4-methoxyphenoxy)-1-methyl-2-oxo-1,2-dihydropyridin-4-yl)-6-methyl-1,6-dihydro-7H-pyrrolo[2,3-c]pyridin-7-one (46 mg, 0.12 mmol) was reacted to give the title compound (27 mg, 59%).

HPLC t_{R} (Agilent, acidic, 3.5 min): 1.13 min, m/z=364.0 [M+H]⁺.

¹H NMR (500 MHz, DMSO) δ 12.03 (bs, 1H), 9.02 (s, 1H), 7.66 (s, 1H), 7.37-7.36 (m, 1H), 7.30 (t, J=2.7 Hz, 1H), 6.65-6.62 (m, 2H), 6.58-6.55 (m, 2H), 6.49 (d, J=13.6 Hz, 1H), 6.32 (t, J=2.3 Hz, 1H), 3.49 (s, 3H), 3.45 (s, 3H).

### Example 27: 5′-(4-methoxyphenoxy)-1,1′-dimethyl-[3,4′-bipyridine]-2′,6(1H, 1′H)-dione Preparation 111: 5′-(4-methoxyphenoxy)-1,1′-dimethyl-[3,4′-bipyridine]-2′,6(1H, 1′H)-dione

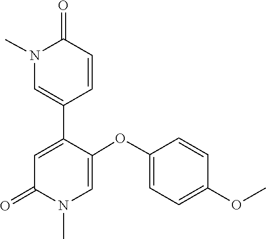

Following the procedure in preparation 40, 4-bromo-5-(4-methoxyphenoxy)-1-methylpyridin-2 (1H)-one (50 mg, 0.16 mmol) was reacted to give the title compound (31 mg, 53%).

HPLC t_{R} (Agilent, acidic, 3.5 min): 1.22 min, m/z=339.0 [M+H]⁺.

¹H NMR (400 MHz, CDCl₃) δ 7.67 (d, J=2.6 Hz, 1H), 7.56 (dd, J=2.7, 9.5 Hz, 1H), 7.06 (s, 1H), 6.82-6.81 (m, 4H), 6.60-6.54 (m, 2H), 3.79-3.78 (m, 3H), 3.53 (s, 6H).

### Example 28: 5′-(3-hydroxyphenoxy)-1,1′-dimethyl-[3,4′-bipyridine]-2′,6(1H, 1′H)-dione Preparation 112: 5′-(3-hydroxyphenoxy)-1,1′-dimethyl-[3,4′-bipyridine]-2′,6(1H, 1′H)-dione

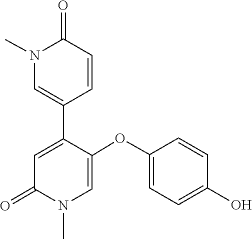 [SMILES: Cn1cc(-c2cc(=O)n(C)cc2Oc2ccc(O)cc2)ccc1=O]

Following the procedure in preparation 11, 5′-(4-methoxyphenoxy)-1,1′-dimethyl-[3,4′-bipyridine]-2′,6 (1H, 1′H)-dione (25 mg, 0.07 mmol) was reacted to give the title compound (12 mg, 49%).

HPLC t_{R} (Agilent, acidic, 3.5 min): 1.08 min, m/z=325.0 [M+H]⁺.

¹H NMR (500 MHz, DMSO-d₆) δ 9.10 (bs, 1H), 8.04 (d, J=2.6 Hz, 1H), 7.66-7.61 (m, 2H), 6.75-6.72 (m, 2H), 6.67-6.64 (m, 2H), 6.51 (s, 1H), 6.35 (d, J=9.5 Hz, 1H), 3.42 (s, 3H), 3.41 (s, 3H).

### Example 29: 5′-(3-hydroxyphenoxy)-1,1′-dimethyl-[3,4′-bipyridine]-2′,6(1H, 1′H)-dione Preparation 113: 5′-(3-methoxyphenoxy)-1,1′-dimethyl-[3,4′-bipyridine]-2′,6(1H, 1′H)-dione

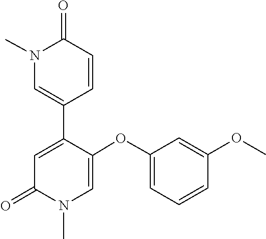

Following the procedure in preparation 40, 4-bromo-5-(3-methoxyphenoxy)-1-methylpyridin-2 (1H)-one (50 mg, 0.16 mmol) was reacted to give the title compound (29 mg, 48%).

HPLC t_{R} (Agilent, acidic, 3.5 min): 1.23 min, m/z=339.0 [M+H]⁺.

### Preparation 114: 5′-(3-hydroxyphenoxy)-1,1′-dimethyl-[3,4′-bipyridine]-2′,6(1H, 1′H)-dione

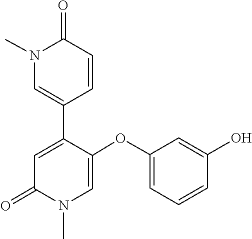 [SMILES: Cn1cc(-c2cc(=O)n(C)cc2Oc2cccc(O)c2)ccc1=O]

Following the procedure in preparation 11, 5′-(4-methoxyphenoxy)-1,1′-dimethyl-[3,4′-bipyridine]-2′,6 (1H, 1′H)-dione (23 mg, 0.07 mmol) was reacted to give the title compound (13 mg, 53%).

HPLC t_{R} (Agilent, acidic, 3.5 min): 1.08 min, m/z=325.0 [M+H]⁺.

¹H NMR (500 MHz, DMSO-d₆) δ 9.48 (bs, 1H), 8.06-8.05 (m, 1H), 7.83-7.82 (m, 1H), 7.62-7.59 (m, 1H), 7.04 (t, J=8.2 Hz, 1H), 6.54 (s, 1H), 6.40-6.25 (m, 4H), 3.44 (s, 3H), 3.41 (s, 3H).

### Example 30: 4-(3-(2-methoxyphenoxy)-1-methyl-2-oxo-1,2-dihydropyridin-4-yl)-6-methyl-1,6-dihydro-7H-pyrrolo[2,3-c]pyridin-7-one

### Preparation 115: 2-chloro-3-(2-methoxyphenoxy)-4-nitropyridine 1-oxide

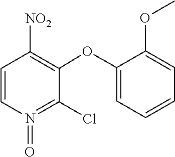 [SMILES: COc1ccccc1Oc1c([N+](=O)[O-])cc[n+]([O-])c1Cl]

Following the procedure in preparation 35, 2-methoxyphenol (6.45 g, 51.9 mmol) and 2-chloro-3-fluoro-4-nitropyridine 1-oxide (10.0 g, 51.9 mmol) was reacted to give the title compound (12.0 g, 78%).

¹H NMR (400 MHz, DMSO-d₆) δ 8.59 (d, J=7.6 Hz, 1H), 8.20 (d, J=7.6 Hz, 1H), 7.17-7.10 (m, 2H), 6.94-6.82 (m, 2H), 3.83 (s, 3H).

### Preparation 116: 2,4-dibromo-3-(2-methoxyphenoxy)pyridine 1-oxide

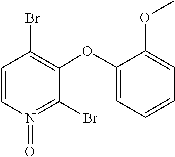 [SMILES: COc1ccccc1Oc1c(Br)cc[n+]([O-])c1Br]

Following the procedure in preparation 26, 2-chloro-3-(2-methoxyphenoxy)-4-nitropyridine 1-oxide (12.0 g, 40.5 mmol) was reacted to give the title compound (11.8 g, 82%).

HPLC t_{R} (Shimadzu, acidic, 1.5 min): 0.83 min, m/z=376.1 [M+H]⁺.

### Preparation 117: 2,4-dibromo-3-(2-methoxyphenoxy)pyridine

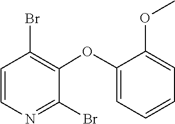 [SMILES: COc1ccccc1Oc1c(Br)ccnc1Br]

Following the procedure in preparation 27, 2,4-dibromo-3-(2-methoxyphenoxy)pyridine 1-oxide (18.0 g, 48.0 mmol) was reacted to give the title compound (14.0 g, 68%).

HPLC t_{R} (Shimadzu, acidic, 1.5 min): 0.94 min, m/z=360.1 [M+H]⁺.

### Preparation 118: 4-bromo-3-(2-methoxyphenoxy)pyridin-2 (1H)-one

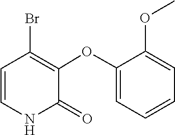 [SMILES: COc1ccccc1Oc1c(Br)cc[nH]c1=O]

Following the procedure in preparation 38, 2,4-dibromo-3-(2-methoxyphenoxy)pyridine (14.0 g, 39.0 mmol) was reacted to give the title compound (2.5 g, 20%).

¹H NMR (400 MHz, DMSO-d₆) δ 12.1 (brs, 1H), 7.26 (d, J=6.8 Hz, 1H), 7.06-7.04 (m, 1H), 6.99-6.78 (m, 2H), 6.53-6.50 (m, 2H), 3.82 (s, 3H).

### Preparation 119: 4-bromo-3-(2-methoxyphenoxy)-1-methylpyridin-2 (1H)-one

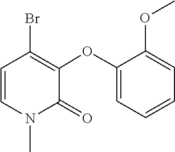 [SMILES: COc1ccccc1Oc1c(Br)ccn(C)c1=O]

Following the procedure in preparation 39, 4-bromo-3-(2-methoxyphenoxy)pyridin-2 (1H)-one (2.4 g, 8.1 mmol) was reacted to give the title compound (1.2 g, 46%).

¹H NMR (400 MHz, DMSO-d₆) δ 7.62 (d, J=7.2 Hz, 1H), 7.08-7.03 (m, 1H), 7.01-6.94 (m, 1H), 6.81-6.74 (m, 1H), 6.58 (d, J=7.2 Hz, 1H), 6.54-6.48 (m, 1H), 3.82 (s, 3H), 3.43 (s, 3H).

### Preparation 120: 4-(3-(2-methoxyphenoxy)-1-methyl-2-oxo-1,2-dihydropyridin-4-yl)-6-methyl-1,6-dihydro-7H-pyrrolo[2,3-c]pyridin-7-one

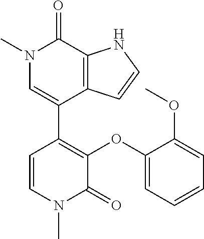 [SMILES: COc1ccccc1Oc1c(-c2cn(C)c(=O)c3[nH]ccc23)ccn(C)c1=O]

Following the procedure in preparation 10, 4-bromo-3-(2-methoxyphenoxy)-1-methylpyridin-2 (1H)-one (30 mg, 0.10 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (41 mg, 0.10 mmol) was reacted to give the title compound (13 mg, 33%).

HPLC t_{R} (Agilent, acidic, 3.5 min): 1.20 min, m/z=378.1 [M+H]⁺.

¹H NMR (400 MHz, DMSO-d₆) δ 12.08 (bs, 1H), 7.68 (d, J=7.2 Hz, 1H), 7.41 (s, 1H), 7.29 (t, J=2.7 Hz, 1H), 6.95 (dd, J=1.5, 8.1 Hz, 1H), 6.87-6.82 (m, 1H), 6.71-6.66 (m, 1H), 6.50-6.41 (m, 2H), 6.31 (t, J=2.3 Hz, 1H), 3.76 (s, 3H), 3.51 (s, 3H), 3.45 (s, 3H).

### Example 31: 4-(3-(2-hydroxyphenoxy)-1-methyl-2-oxo-1,2-dihydropyridin-4-yl)-6-methyl-1,6-dihydro-7H-pyrrolo[2,3-c]pyridin-7-one

### Preparation 121: 4-(3-(2-hydroxyphenoxy)-1-methyl-2-oxo-1,2-dihydropyridin-4-yl)-6-methyl-1,6-dihydro-7H-pyrrolo[2,3-c]pyridin-7-one

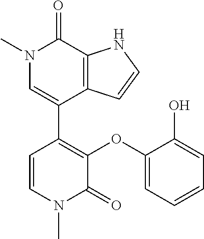 [SMILES: Cn1ccc(-c2cn(C)c(=O)c3[nH]ccc23)c(Oc2ccccc2O)c1=O]

Following the procedure in preparation 11, 4-(3-(2-methoxyphenoxy)-1-methyl-2-oxo-1,2-dihydropyridin-4-yl)-6-methyl-1,6-dihydro-7H-pyrrolo[2,3-c]pyridin-7-one (66 mg, 0.18 mmol) was reacted to give the title compound (17 mg, 26%).

HPLC t_{R} (Agilent, acidic, 3.5 min): 1.16 min, m/z=364.1 [M+H]⁺.

¹H NMR (500 MHz, DMSO-d₆) δ 12.17 (bs, 1H), 9.38 (s, 1H), 7.72 (d, J=7.2 Hz, 1H), 7.64 (s, 1H), 7.32 (t, J=2.8 Hz, 1H), 6.78-6.72 (m, 2H), 6.53-6.47 (m, 2H), 6.38-6.35 (m, 2H), 3.54 (s, 3H), 3.17 (s, 3H).

### Example 32: 4-(3-(3-methoxyphenoxy)-1-methyl-2-oxo-1,2-dihydropyridin-4-yl)-6-methyl-1,6-dihydro-7H-pyrrolo[2,3-c]pyridin-7-one

### Preparation 122: 2-chloro-3-(3-methoxyphenoxy)-4-nitropyridine 1-oxide

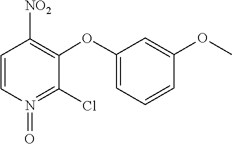 [SMILES: COc1cccc(Oc2c([N+](=O)[O-])cc[n+]([O-])c2Cl)c1]

Following the procedure in preparation 35, 3-methoxyphenol (6.45 g, 51.9 mmol) and 2-chloro-3-fluoro-4-nitropyridine 1-oxide (10.0 g, 51.9 mmol) was reacted to give the title compound (9.0 g, 58%).

¹H NMR (400 MHz, DMSO-d₆) δ 8.61 (d, J=7.6 Hz, 1H), 8.22 (d, J=7.2 Hz, 1H), 7.27-7.23 (m, 1H), 6.74-6.61 (m, 3H), 3.74 (s, 3H).

### Preparation 123: 2,4-dibromo-3-(3-methoxyphenoxy)pyridine 1-oxide

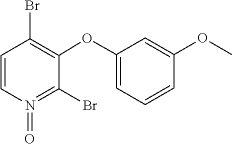 [SMILES: COc1cccc(Oc2c(Br)cc[n+]([O-])c2Br)c1]

Following the procedure in preparation 26, 2-chloro-3-(3-methoxyphenoxy)-4-nitropyridine 1-oxide (9.0 g, 30.3 mmol) was reacted to give the title compound (11.0 g, 97%).

HPLC t_{R} (Shimadzu, acidic, 1.5 min): 0.85 min, m/z=376.0 [M]⁺.

### Preparation 124: 2,4-dibromo-3-(3-methoxyphenoxy)pyridine

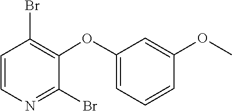 [SMILES: COc1cccc(Oc2c(Br)ccnc2Br)c1]

Following the procedure in preparation 27, 2,4-dibromo-3-(3-methoxyphenoxy)pyridine 1-oxide (11.0 g, 29.3 mmol) was reacted to give the title compound (10.1 g, 96%).

HPLC t_{R} (Agilent, acidic, 1.5 min): 0.97 min, m/z=359.8 [M+H]⁺.

### Preparation 125: 4-bromo-3-(3-methoxyphenoxy)pyridin-2 (1H)-one

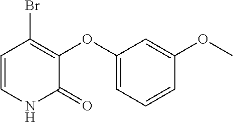 [SMILES: COc1cccc(Oc2c(Br)cc[nH]c2=O)c1]

Following the procedure in preparation 38, 2,4-dibromo-3-(3-methoxyphenoxy)pyridine (10.0 g, 27.9 mmol) was reacted to give the title compound (1.0 g, 12%).

¹H NMR (400 MHz, DMSO-d₆) δ 7.34 (d, J=5.6 Hz, 1H), 7.13-7.09 (m, 1H), 6.52-6.21 (m, 4H), 3.70 (s, 3H).

### Preparation 126: 4-bromo-3-(3-methoxyphenoxy)-1-methylpyridin-2 (1H)-one

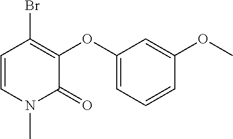 [SMILES: COc1cccc(Oc2c(Br)ccn(C)c2=O)c1]

Following the procedure in preparation 39, 4-bromo-3-(3-methoxyphenoxy)pyridin-2 (1H)-one (1.0 g, 3.38 mmol) was reacted to give the title compound (0.7 g, 66%).

¹H NMR (400 MHz, DMSO-d₆) δ 7.65 (d, J=7.2 Hz, 1H), 7.17 (t, J=8.4 Hz, 1H), 6.64-6.58 (m, 2H), 6.43 (t, J=2.4 Hz, 1H), 6.38-6.34 (m, 1H), 3.72 (s, 3H), 3.45 (s, 3H). Preparation 127: 4-(3-(3-methoxyphenoxy)-1-methyl-2-oxo-1,2-dihydropyridin-4-yl)-6-methyl-1,6-dihydro-7H-pyrrolo[2,3-c]pyridin-7-one

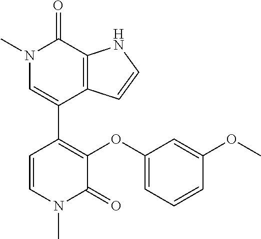

Following the procedure in preparation 10, 4-bromo-3-(3-methoxyphenoxy)-1-methylpyridin-2 (1H)-one (30 mg, 0.10 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (39 mg, 0.092 mmol) was reacted to give the title compound (12 mg, 30%).

HPLC t_{R} (Agilent, acidic, 3.5 min): 1.19 min, m/z=378.1 [M+H]⁺.

¹H NMR (400 MHz, DMSO-d₆) δ 12.07 (s, 1H), 7.71 (d, J=7.1 Hz, 1H), 7.36 (s, 1H), 7.32 (t, J=2.7 Hz, 1H), 7.06 (t, J=8.1 Hz, 1H), 6.48 (dd, J=2.0, 7.9 Hz, 1H), 6.40 (d, J=7.1 Hz, 1H), 6.32-6.25 (m, 3H), 3.64 (s, 3H), 3.52 (s, 3H), 3.47 (s, 3H).

### Example 33: 4-(3-(3-hydroxyphenoxy)-1-methyl-2-oxo-1,2-dihydropyridin-4-yl)-6-methyl-1,6-dihydro-7H-pyrrolo[2,3-c]pyridin-7-one

### Preparation 128: 4-(3-(3-hydroxyphenoxy)-1-methyl-2-oxo-1,2-dihydropyridin-4-yl)-6-methyl-1,6-dihydro-7H-pyrrolo[2,3-c]pyridin-7-one

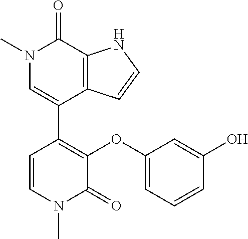 [SMILES: Cn1ccc(-c2cn(C)c(=O)c3[nH]ccc23)c(Oc2cccc(O)c2)c1=O]

Following the procedure in preparation 11, 4-(3-(3-methoxyphenoxy)-1-methyl-2-oxo-1,2-dihydropyridin-4-yl)-6-methyl-1,6-dihydro-7H-pyrrolo[2,3-c]pyridin-7-one (66 mg, 0.18 mmol) was reacted to give the title compound (15 mg, 23%).

HPLC t_{R} (Agilent, acidic, 3.5 min): 1.12 min, m/z=364.1 [M+H]⁺.

¹H NMR (500 MHz, DMSO-d₆) & 12.15 (bs, 1H), 9.34 (s, 1H), 7.71 (d, J=7.0 Hz, 1H), 7.37-7.31 (m, 2H), 6.93 (t, J=8.1 Hz, 1H), 6.41 (d, J=7.0 Hz, 1H), 6.32-6.28 (m, 2H), 6.16-6.09 (m, 2H), 3.52 (s, 3H), 3.47 (s, 3H).

### Example 34: 4-(3-(4-methoxyphenoxy)-1-methyl-2-oxo-1,2-dihydropyridin-4-yl)-6-methyl-1,6-dihydro-7H-pyrrolo[2,3-c]pyridin-7-one

### Preparation 129: 2-chloro-3-(4-methoxyphenoxy)-4-nitropyridine 1-oxide

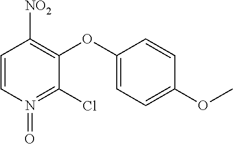 [SMILES: COc1ccc(Oc2c([N+](=O)[O-])cc[n+]([O-])c2Cl)cc1]

Following the procedure in preparation 35, 4-methoxyphenol (6.5 g, 52.4 mmol) and 2-chloro-3-fluoro-4-nitropyridine 1-oxide (10.0 g, 51.9 mmol) was reacted to give the title compound (12.0 g, 78%).

¹H NMR (400 MHz, DMSO-d₆) δ 8.60 (d, J=7.6 Hz, 1H), 8.20 (d, J=7.2 Hz, 1H), 7.05-6.99 (m, 2H), 6.92-6.87 (m, 2H), 3.73 (s, 3H).

### Preparation 130: 2,4-dibromo-3-(4-methoxyphenoxy)pyridine 1-oxide

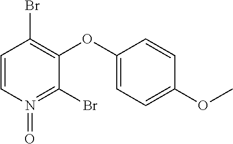 [SMILES: COc1ccc(Oc2c(Br)cc[n+]([O-])c2Br)cc1]

Following the procedure in preparation 26, 2-chloro-3-(4-methoxyphenoxy)-4-nitropyridine 1-oxide (12.0 g, 40.5 mmol) was reacted to give the title compound (12.1 g, 79%).

HPLC t_{R} (Shimadzu, acidic, 1.5 min): 0.85 min, m/z=375.9 [M+H]⁺.

### Preparation 131: 2,4-dibromo-3-(4-methoxyphenoxy)pyridine

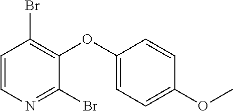 [SMILES: COc1ccc(Oc2c(Br)ccnc2Br)cc1]

Following the procedure in preparation 27, 2,4-dibromo-3-(4-methoxyphenoxy)pyridine 1-oxide (18.0 g, 48.0 mmol) was reacted to give the title compound (14.0 g, 75%).

HPLC t_{R} (Shimadzu, acidic, 1.5 min): 0.97 min, m/z=360.1 [M+H]⁺.

### Preparation 132: 4-bromo-3-(4-methoxyphenoxy)pyridin-2 (1H)-one

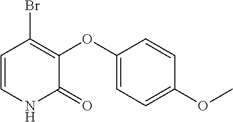 [SMILES: COc1ccc(Oc2c(Br)cc[nH]c2=O)cc1]

Following the procedure in preparation 38, 2,4-dibromo-3-(4-methoxyphenoxy)pyridine (14.0 g, 39.0 mmol) was reacted to give the title compound (2.0 g, 17%).

¹H NMR (400 MHz, DMSO-d₆) δ 7.28 (d, J=6.8 Hz, 1H), 6.86-6.81 (m, 2H), 6.80-6.75 (m, 2H), 6.46 (d, J=6.8 Hz, 1H), 3.70 (s, 3H).

### Preparation 133: 4-bromo-3-(4-methoxyphenoxy)-1-methylpyridin-2 (1H)-one

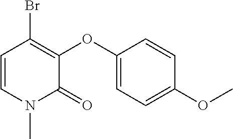 [SMILES: COc1ccc(Oc2c(Br)ccn(C)c2=O)cc1]

Following the procedure in preparation 39, 4-bromo-3-(4-methoxyphenoxy)pyridin-2 (1H)-one (2.0 g, 6.8 mmol) was reacted to give the title compound (1.8 g, 82%).

¹H NMR (400 MHz, DMSO-d₆) δ 7.62 (d, J=7.6 Hz, 1H), 6.86-6.82 (m, 2H), 6.81-6.75 (m, 2H), 6.57 (d, J=7.2 Hz, 1H), 3.70 (s, 3H), 3.44 (s, 3H)

### Preparation 134: 4-(3-(4-methoxyphenoxy)-1-methyl-2-oxo-1,2-dihydropyridin-4-yl)-6-methyl-1,6-dihydro-7H-pyrrolo[2,3-c]pyridin-7-one

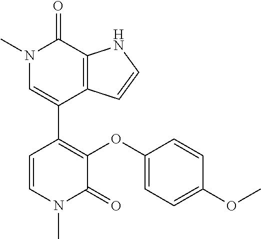

Following the procedure in preparation 10, 4-bromo-3-(4-methoxyphenoxy)-1-methylpyridin-2 (1H)-one (30 mg, 0.10 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (39 mg, 0.092 mmol) was reacted to give the title compound (8 mg, 20%).

HPLC t_{R} (Agilent, acidic, 3.5 min): 1.17 min, m/z=378.1 [M+H]⁺.

¹H NMR (400 MHz, DMSO-d₆) & 12.08 (s, 1H), 7.68 (d, J=7.2 Hz, 1H), 7.36-7.30 (m, 2H), 6.75-6.62 (m, 4H), 6.39 (d, J=7.1 Hz, 1H), 6.30 (t, J=2.3 Hz, 1H), 3.64 (s, 3H), 3.51 (s, 3H), 3.48 (s, 3H).

### Example 35: 4-(3-(4-hydroxyphenoxy)-1-methyl-2-oxo-1,2-dihydropyridin-4-yl)-6-methyl-1,6-dihydro-7H-pyrrolo[2,3-c]pyridin-7-one

### Preparation 135: 4-(3-(4-hydroxyphenoxy)-1-methyl-2-oxo-1,2-dihydropyridin-4-yl)-6-methyl-1,6-dihydro-7H-pyrrolo[2,3-c]pyridin-7-one

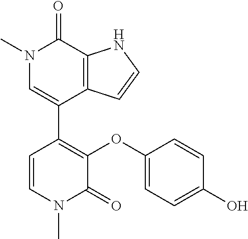 [SMILES: Cn1ccc(-c2cn(C)c(=O)c3[nH]ccc23)c(Oc2ccc(O)cc2)c1=O]

Following the procedure in preparation 11, 4-(3-(4-methoxyphenoxy)-1-methyl-2-oxo-1,2-dihydropyridin-4-yl)-6-methyl-1,6-dihydro-7H-pyrrolo[2,3-c]pyridin-7-one (63 mg, 0.17 mmol) was reacted to give the title compound (17 mg, 27%).

HPLC t_{R} (Agilent, acidic, 3.5 min): 1.06 min, m/z=364.0 [M+H]⁺.

¹H NMR (500 MHz, DMSO-d₆) & 12.12 (bs, 1H), 8.93 (s, 1H), 7.68 (d, J=7.2 Hz, 1H), 7.35 (s, 1H), 7.32 (t, J=2.7 Hz, 1H), 6.54-6.52 (m, 4H), 6.38 (d, J=7.0 Hz, 1H), 6.29 (t, J=2.2 Hz, 1H), 3.51 (s, 3H), 3.47 (s, 3H).

### Example 36: 3′-(2-hydroxyphenoxy)-1,1′-dimethyl-[3,4′-bipyridine]-2′,6(1H, 1′H)-dione

### Preparation 136: 3′-(2-methoxyphenoxy)-1,1′-dimethyl-[3,4′-bipyridine]-2′,6(1H, 1′H)-dione

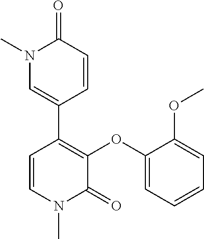

Following the procedure in preparation 40, 4-bromo-3-(2-methoxyphenoxy)-1-methylpyridin-2 (1H)-one (100 mg, 0.32 mmol) was reacted to give the title compound (37 mg, 34%).

HPLC t_{R} (Agilent, acidic, 3.5 min): 1.15 min, m/z=339.0 [M+H]⁺.

### Preparation 137: 5′-(2-hydroxyphenoxy)-1,1′-dimethyl-[3,4′-bipyridine]-2′,6(1H, 1′H)-dione

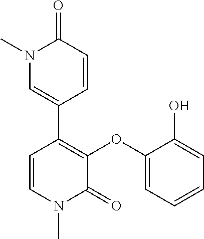 [SMILES: Cn1cc(-c2ccn(C)c(=O)c2Oc2ccccc2O)ccc1=O]

Following the procedure in preparation 11, 3′-(2-methoxyphenoxy)-1,1′-dimethyl-[3,4′-bipyridine]-2′,6 (1H, 1′H)-dione (37 mg, 0.11 mmol) was reacted to give the title compound (21 mg, 53%).

HPLC t_{R} (Agilent, acidic, 3.5 min): 1.10 min, m/z=325.0 [M+H]⁺.

¹H NMR (500 MHz, DMSO-d₆) δ 9.38 (s, 1H), 8.24 (d, J=2.4 Hz, 1H), 7.76 (dd, J=2.6, 10.0 Hz, 1H), 7.71 (d, J=7.2 Hz, 1H), 6.84-6.77 (m, 2H), 6.60-6.56 (m, 1H), 6.46 (d, J=6.9 Hz, 1H), 6.42-6.37 (m, 2H), 3.50 (s, 3H), 3.46 (s, 3H).

### Example 37: 3′-(3-hydroxyphenoxy)-1,1′-dimethyl-[3,4′-bipyridine]-2′,6(1H, 1′H)-dione

### Preparation 138: 3′-(3-methoxyphenoxy)-1,1′-dimethyl-[3,4′-bipyridine]-2′,6(1H, 1′H)-dione

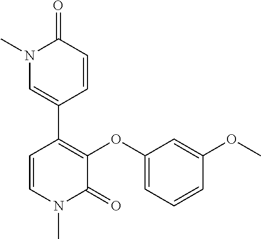

Following the procedure in preparation 40, 4-bromo-3-(3-methoxyphenoxy)-1-methylpyridin-2 (1H)-one (100 mg, 0.32 mmol) was reacted to give the title compound (58 mg, 53%).

HPLC t_{R} (Agilent, acidic, 3.5 min): 1.17 min, m/z=339.0 [M+H]⁺.

### Preparation 139: 5′-(3-hydroxyphenoxy)-1,1′-dimethyl-[3,4′-bipyridine]-2′,6(1H, 1′H)-dione

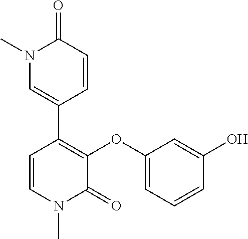 [SMILES: Cn1cc(-c2ccn(C)c(=O)c2Oc2cccc(O)c2)ccc1=O]

Following the procedure in preparation 11, 3′-(3-methoxyphenoxy)-1,1′-dimethyl-[3,4′-bipyridine]-2′,6(1H, 1′H)-dione (58 mg, 0.17 mmol) was reacted to give the title compound (23 mg, 40%).

HPLC t_{R} (Agilent, acidic, 3.5 min): 1.05 min, m/z=325.0 [M+H]⁺.

¹H NMR (500 MHz, DMSO-d₆) δ 9.43 (s, 1H), 8.10 (d, J=2.6 Hz, 1H), 7.71 (d, J=7.2 Hz, 1H), 7.63 (dd, J=2.7, 9.5 Hz, 1H), 7.02 (t, J=8.2 Hz, 1H), 6.43 (d, J=7.2 Hz, 1H), 6.40-6.37 (m, 2H), 6.24 (dd, J=2.3, 8.1 Hz, 1H), 6.17 (t, J=2.2 Hz, 1H), 3.48 (s, 3H), 3.45 (s, 3H).

### Example 38: 3′-(4-hydroxyphenoxy)-1,1′-dimethyl-[3,4′-bipyridine]-2′,6(1H, 1′H)-dione Preparation 140: 3′-(4-methoxyphenoxy)-1,1′-dimethyl-[3,4′-bipyridine]-2′,6(1H, 1′H)-dione

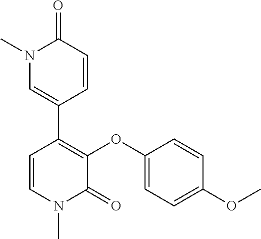

Following the procedure in preparation 40, 4-bromo-3-(4-methoxyphenoxy)-1-methylpyridin-2 (1H)-one (100 mg, 0.32 mmol) was reacted to give the title compound (50 mg, 46%).

HPLC t_{R} (Agilent, acidic, 3.5 min): 1.15 min, m/z=339.0 [M+H]⁺.

### Preparation 141: 5′-(4-hydroxyphenoxy)-1,1′-dimethyl-[3,4′-bipyridine]-2′,6(1H, 1′H)-dione

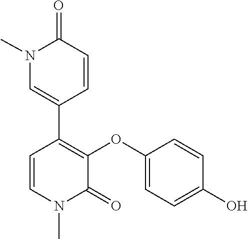 [SMILES: Cn1cc(-c2ccn(C)c(=O)c2Oc2ccc(O)cc2)ccc1=O]

Following the procedure in preparation 11, 3′-(4-methoxyphenoxy)-1,1′-dimethyl-[3,4′-bipyridine]-2′,6(1H, 1′H)-dione (50 mg, 0.15 mmol) was reacted to give the title compound (18 mg, 34%).

HPLC t_{R} (Agilent, acidic, 3.5 min): 1.00 min, m/z=325.0 [M+H]⁺.

¹H NMR (500 MHz, DMSO-d₆) δ 9.02 (m, 1H), 8.08 (d, J=2.6 Hz, 1H), 7.69-7.62 (m, 2H), 6.62-6.61 (m, 4H), 6.39 (dd, J=8.4, 10.9 Hz, 2H), 3.46 (s, 3H), 3.45 (s, 3H).

### Example 39: 3′-(4-fluoro-2,6-dimethylphenoxy)-1,1′-dimethyl-[3,4′-bipyridine]-2′,6(1H, 1′H)-dione Preparation 142: 2-chloro-3-(4-fluoro-2,6-dimethylphenoxy)-4-nitropyridine 1-oxide

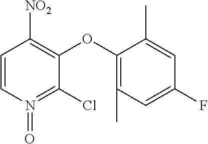 [SMILES: Cc1cc(F)cc(C)c1O/C(CCl)=C(/C=C\N=O)[N+](=O)[O-]]

Following the procedure in preparation 35, 4-fluoro-2,6-dimethylphenol (14.0 g, 99.9 mmol) and 2-chloro-3-fluoro-4-nitropyridine 1-oxide (10.0 g, 51.9 mmol) was reacted to give the title compound (11.0 g, 47%).

HPLC t_{R} (Shimadzu, acidic, 1.5 min): 0.94 min, m/z=313.2 [M+H]⁺.

### Preparation 143: 2,4-dibromo-3-(4-fluoro-2,6-dimethylphenoxy)pyridine 1-oxide

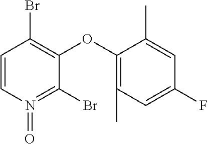 [SMILES: Cc1cc(F)cc(C)c1O/C(CBr)=C(Br)/C=C\N=O]

Following the procedure in preparation 26, 2-chloro-3-(4-fluoro-2,6-dimethylphenoxy)-4-nitropyridine 1-oxide (10.0 g, 31.9 mmol) was reacted to give the title compound (11.6 g, 93%).

HPLC t_{R} (Shimadzu, acidic, 1.5 min): 0.92 min, m/z=392.0 [M+H]⁺.

### Preparation 144: 2,4-dibromo-3-(4-fluoro-2,6-dimethylphenoxy)pyridine

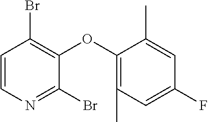 [SMILES: Cc1cc(F)cc(C)c1Oc1c(Br)ccnc1Br]

Following the procedure in preparation 27, 2,4-dibromo-3-(4-fluoro-2,6-dimethylphenoxy)pyridine 1-oxide (15.0 mg, 38.4 mmol) was reacted to give the title compound (12.2 g, 85%).

HPLC t_{R} (Shimadzu, acidic, 1.5 min): 1.15 min, m/z=376.1 [M+H]⁺.

### Preparation 145: 4-bromo-3-(4-fluoro-2,6-dimethylphenoxy)pyridin-2 (1H)-one

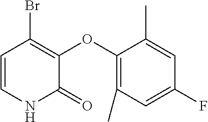 [SMILES: Cc1cc(F)cc(C)c1Oc1c(Br)cc[nH]c1=O]

Following the procedure in preparation 38, 2,4-dibromo-3-(4-fluoro-2,6-dimethylphenoxy)pyridine (11.6 g, 30.9 mmol) was reacted to give the title compound (8.0 g, 83%).

HPLC t_{R} (Shimadzu, acidic, 1.5 min): 0.88 min, m/z=313.8 [M+H]⁺.

### Preparation 146: 4-bromo-3-(4-fluoro-2,6-dimethylphenoxy)pyridin-2 (1H)-one

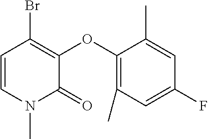 [SMILES: Cc1cc(F)cc(C)c1Oc1c(Br)ccn(C)c1=O]

Following the procedure in preparation 39, 4-bromo-3-(4-fluoro-2,6-dimethylphenoxy)pyridin-2 (1H)-one (7.5 g, 24.0 mmol) was reacted to give the title compound (1.0 g, 13%).

¹H NMR (400 MHz, DMSO-d₆) δ 7.46 (d, J=7.2 Hz, 1H), 6.83 (d, J=9.2 Hz, 2H), 6.55 (d, J=7.2 Hz, 1H), 3.35 (s, 3H), 2.09 (s, 6H)

### Preparation 147: 3′-(4-fluoro-2,6-dimethylphenoxy)-1,1′-dimethyl-[3,4′-bipyridine]-2′,6(1H, 1′H)-dione

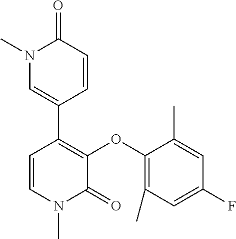 [SMILES: Cc1cc(F)cc(C)c1Oc1c(-c2ccc(=O)n(C)c2)ccn(C)c1=O]

Following the procedure in preparation 40, 4-bromo-3-(4-fluoro-2,6-dimethylphenoxy)pyridin-2 (1H)-one (100 mg, 0.31 mmol) was reacted to give the title compound (73 mg, 60%).

HPLC t_{R} (Agilent, acidic, 3.5 min): 1.34 min, m/z=355.0 [M+H]⁺.

¹H NMR (500 MHz, DMSO-d₆) δ 8.11-8.09 (m, 1H), 7.80 (dd, J=2.7, 9.5 Hz, 1H), 7.53 (d, J=7.2 Hz, 1H), 6.78-6.75 (m, 2H), 6.45 (d, J=9.5 Hz, 1H), 6.36 (d, J=7.2 Hz, 1H), 3.51-3.50 (m, 3H), 3.40 (s, 3H), 2.04-2.03 (m, 6H).

### Example 40: 4-(3-(4-fluoro-2,6-dimethylphenoxy)-1-methyl-2-oxo-1,2-dihydropyridin-4-yl)-6-methyl-1,6-dihydro-7H-pyrrolo[2,3-c]pyridin-7-one

### Preparation 148: 4-(3-(4-fluoro-2,6-dimethylphenoxy)-1-methyl-2-oxo-1,2-dihydropyridin-4-yl)-6-methyl-1,6-dihydro-7H-pyrrolo[2,3-c]pyridin-7-one

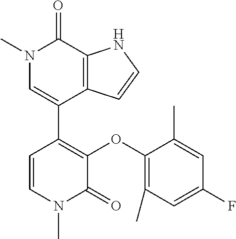 [SMILES: Cc1cc(F)cc(C)c1Oc1c(-c2cn(C)c(=O)c3[nH]ccc23)ccn(C)c1=O]

Following the procedure in preparation 10, 4-bromo-3-(4-fluoro-2,6-dimethylphenoxy)pyridin-2 (1H)-one (100 mg, 0.31 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (124 mg, 0.29 mmol) was reacted to give the title compound (50 mg, 40%).

HPLC t_{R} (Agilent, acidic, 3.5 min): 1.36 min, m/z=395.1 [M+H]⁺.

¹H NMR (500 MHz, DMSO-d₆) δ 12.07 (s, 1H), 7.54 (d, J=7.0 Hz, 1H), 7.36 (s, 1H), 7.31 (t, J=2.7 Hz, 1H), 6.69-6.66 (m, 2H), 6.32-6.26 (m, 2H), 3.55 (s, 3H), 3.44 (s, 3H), 2.01-2.00 (m, 6H).

### Example 41: N-ethyl-4-(3-(4-fluoro-2,6-dimethylphenoxy)-1-methyl-2-oxo-1,2-dihydropyridin-4-yl)-6-methyl-7-oxo-6,7-dihydro-1H-pyrrolo[2,3-c]pyridine-2-carboxamide

### Preparation 149: ethyl 4-bromo-6-methyl-7-oxo-1-tosyl-6,7-dihydro-1H-pyrrolo[2,3-c]pyridine-2-carboxylate

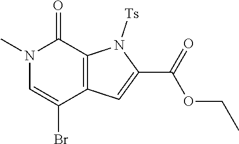 [SMILES: CCOC(=O)c1cc2c(Br)cn(C)c(=O)c2n1S(=O)(=O)c1ccc(C)cc1]

4-bromo-6-methyl-1-tosyl-1,6-dihydro-7H-pyrrolo[2,3-c]pyridin-7-one (1.3 g, 3.4 mmol), in THF (100 mL) was cooled to −78 °C. LDA (2.03 mL, 4.06 mmol) was added dropwise and the resulting solution stirred at this temperature for 30 minutes. Ethyl carbonochloridate (0.39 mL, 4.06 mmol) was added and the reaction stirred for 1 hour at −78 °C. Ethyl acetate (500 ml) was added and the organics washed with 2×500 ml water then 1×500 ml saturated brine solution. The organics were then separated and dried (MgSO₄) before concentration to dryness. The crude was then purified by flash column chromatography eluting with ethyl acetate/heptane gradient (0-100%). The desired fractions were combined and dried to afford was reacted to give the title compound (770 mg, 50%).

HPLC t_{R} (Agilent, acidic, 3.5 min): 1.85 min, m/z=454.8 [M+H]⁺.

### Preparation 150: ethyl 6-methyl-7-oxo-4-(4,4,5,5-tetramethyl-1,3,2-dioxaborolan-2-yl)-1-tosyl-6,7-dihydro-1H-pyrrolo[2,3-c]pyridine-2-carboxylate

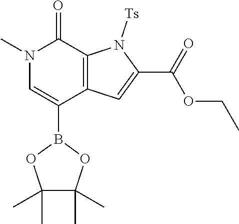 [SMILES: CCOC(=O)c1cc2c(B3OC(C)(C)C(C)(C)O3)cn(C)c(=O)c2n1S(=O)(=O)c1ccc(C)cc1]

Following the procedure in preparation 6, ethyl 4-bromo-6-methyl-7-oxo-1-tosyl-6,7-dihydro-1H-pyrrolo[2,3-c]pyridine-2-carboxylate (710 mg, 1.6 mmol) was reacted to give the title compound (437 mg, 56%).

HPLC t_{R} (Agilent, acidic, 3.5 min): 2.10 min, m/z=501.1 [M+H]⁺.

### Preparation 151: 4-(3-(4-fluoro-2,6-dimethylphenoxy)-1-methyl-2-oxo-1,2-dihydropyridin-4-yl)-6-methyl-7-oxo-6,7-dihydro-1H-pyrrolo[2,3-c]pyridine-2-carboxylic acid

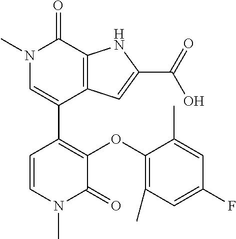 [SMILES: Cc1cc(F)cc(C)c1Oc1c(-c2cn(C)c(=O)c3[nH]c(C(=O)O)cc23)ccn(C)c1=O]

Following the procedure in preparation 10, 4-bromo-3-(4-fluoro-2,6-dimethylphenoxy)pyridin-2 (1H)-one (285 mg, 0.87 mmol) and ethyl 6-methyl-7-oxo-4-(4,4,5,5-tetramethyl-1,3,2-dioxaborolan-2-yl)-1-tosyl-6,7-dihydro-1H-pyrrolo[2,3-c]pyridine-2-carboxylate (436 mg, 0.87 mmol) was reacted to give the title compound (112 mg, 29%).

HPLC t_{R} (Agilent, acidic, 3.5 min): 1.17 min, m/z=378.1 [M+H]⁺.

### Preparation 152: N-ethyl-4-(3-(4-fluoro-2,6-dimethylphenoxy)-1-methyl-2-oxo-1,2-dihydropyridin-4-yl)-6-methyl-7-oxo-6,7-dihydro-1H-pyrrolo[2,3-c]pyridine-2-carboxamide

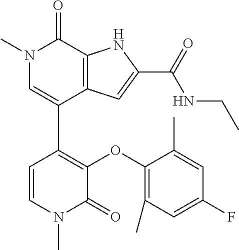 [SMILES: CCNC(=O)c1cc2c(-c3ccn(C)c(=O)c3Oc3c(C)cc(F)cc3C)cn(C)c(=O)c2[nH]1]

To a solution of 4-(3-(4-fluoro-2,6-dimethylphenoxy)-1-methyl-2-oxo-1,2-dihydropyridin-4-yl)-6-methyl-7-oxo-6,7-dihydro-1H-pyrrolo[2,3-c]pyridine-2-carboxylic acid (25 mg, 0.06 mmol) in DCM (1 mL) was added oxalyl chloride (0.1 mL, 0.11 mmol) and DMF (0.01 mL). Reaction was stirred for 1 h at room temperature. Solvent removed under reduced pressure and THF (1 mL) added. 30% ethylamine solution (0.11 mL, 0.23 mmol) in THF was added and the resulting solution stirred for 2 h at room temperature. Ethyl acetate (50 ml) was added and the organics washed with 2×50 ml water then 1×50 ml saturated brine solution. The organics were then separated and dried (MgSO₄) before concentration to dryness. The crude was then purified by flash column chromatography eluting with ethyl acetate/heptane gradient (0-100%). The desired fractions were combined and dried to afford was reacted to give the title compound (12 mg, 42%).

HPLC t_{R} (Agilent, acidic, 3.5 min): 1.52 min, m/z=465.2 [M+H]⁺.

¹H NMR (500 MHz, DMSO-d₆) & 12.25 (bs, 1H), 8.34 (t, J=5.3 Hz, 1H), 7.57 (d, J=7.2 Hz, 1H), 7.41 (s, 1H), 6.90 (s, 1H), 6.70-6.67 (m, 2H), 6.33-6.31 (m, 1H), 3.56 (s, 3H), 3.45 (s, 3H), 3.28-3.30 (m, 2H), 2.00 (s, 6H), 1.14 (t, J=7.2 Hz, 3H).

### Example 42: N-(tert-butyl)-4-(3-(4-fluoro-2,6-dimethylphenoxy)-1-methyl-2-oxo-1,2-dihydropyridin-4-yl)-6-methyl-7-oxo-6,7-dihydro-1H-pyrrolo[2,3-c]pyridine-2-carboxamide

### Preparation 153: N-(tert-butyl)-4-(3-(4-fluoro-2,6-dimethylphenoxy)-1-methyl-2-oxo-1,2-dihydropyridin-4-yl)-6-methyl-7-oxo-6,7-dihydro-1H-pyrrolo[2,3-c]pyridine-2-carboxamide

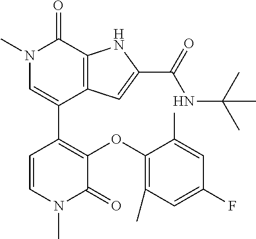 [SMILES: Cc1cc(F)cc(C)c1Oc1c(-c2cn(C)c(=O)c3[nH]c(C(=O)NC(C)(C)C)cc23)ccn(C)c1=O]

Following the procedure in preparation 152, 4-(3-(4-fluoro-2,6-dimethylphenoxy)-1-methyl-2-oxo-1,2-dihydropyridin-4-yl)-6-methyl-7-oxo-6,7-dihydro-1H-pyrrolo[2,3-c]pyridine-2-carboxylic acid (15.6 mg, 0.04 mmol) and 2-amino-2-methylpropane (0.015 mL, 0.14 mmol) was reacted to give the title compound (3 mg, 16%).

HPLC t_{R} (Agilent, acidic, 3.5 min): 1.54 min, m/z=493.2 [M+H]⁺.

¹H NMR (400 MHz, DMSO-d₆) δ 12.36 (bs, 1H), 7.84 (s, 1H), 7.56 (d, J=7.2 Hz, 1H), 7.43 (s, 1H), 6.89 (d, J=1.1 Hz, 1H), 6.71-6.67 (m, 2H), 6.33 (d, J=7.0 Hz, 1H), 3.57 (s, 3H), 3.45 (s, 3H), 2.01-2.00 (m, 6H), 1.39 (s, 9H).

### Example 43: N-(tert-butyl)-4-(3-(4-fluoro-2,6-dimethylphenoxy)-1-methyl-2-oxo-1,2-dihydropyridin-4-yl)-6-methyl-7-oxo-6,7-dihydro-1H-pyrrolo[2,3-c]pyridine-2-carboxamide

### Preparation 154: N-(tert-butyl)-4-(3-(4-fluoro-2,6-dimethylphenoxy)-1-methyl-2-oxo-1,2-dihydropyridin-4-yl)-6-methyl-7-oxo-6,7-dihydro-1H-pyrrolo[2,3-c]pyridine-2-carboxamide

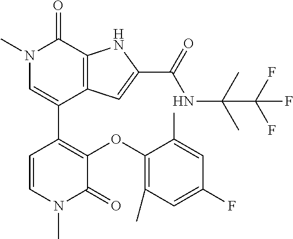 [SMILES: Cc1cc(F)cc(C)c1Oc1c(-c2cn(C)c(=O)c3[nH]c(C(=O)NC(C)(C)C(F)(F)F)cc23)ccn(C)c1=O]

Following the procedure in preparation 152, 4-(3-(4-fluoro-2,6-dimethylphenoxy)-1-methyl-2-oxo-1,2-dihydropyridin-4-yl)-6-methyl-7-oxo-6,7-dihydro-1H-pyrrolo[2,3-c]pyridine-2-carboxylic acid (15.6 mg, 0.04 mmol) and 1,1,1-trifluoro-2-methylpropan-2-amine (18.3 mg, 0.14 mmol) was reacted to give the title compound (5 mg, 23%).

HPLC t_{R} (Agilent, acidic, 3.5 min): 1.60 min, m/z=547.1 [M+H]⁺.

¹H NMR (400 MHz, DMSO-d₆) δ 12.49 (bs, 1H), 8.06 (s, 1H), 7.57 (d, J=7.1 Hz, 1H), 7.44 (s, 1H), 7.00 (d, J=2.2 Hz, 1H), 6.70-6.67 (m, 2H), 6.33 (d, J=7.1 Hz, 1H), 3.57 (s, 3H), 3.45 (s, 3H), 2.01 (s, 6H), 1.63 (s, 6H).

### Example 44: N-(2,2-difluoro-1-methylcyclopropyl)-4-(3-(4-fluoro-2,6-dimethylphenoxy)-1-methyl-2-oxo-1,2-dihydropyridin-4-yl)-6-methyl-7-oxo-6,7-dihydro-1H-pyrrolo[2,3-c]pyridine-2-carboxamide

### Preparation 155: N-(2,2-difluoro-1-methylcyclopropyl)-4-(3-(4-fluoro-2,6-dimethylphenoxy)-1-methyl-2-oxo-1,2-dihydropyridin-4-yl)-6-methyl-7-oxo-6,7-dihydro-1H-pyrrolo[2,3-c]pyridine-2-carboxamide

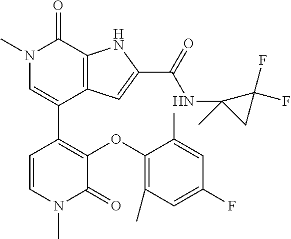 [SMILES: Cc1cc(F)cc(C)c1Oc1c(-c2cn(C)c(=O)c3[nH]c(C(=O)NC4(C)CC4(F)F)cc23)ccn(C)c1=O]

Following the procedure in preparation 152, 4-(3-(4-fluoro-2,6-dimethylphenoxy)-1-methyl-2-oxo-1,2-dihydropyridin-4-yl)-6-methyl-7-oxo-6,7-dihydro-1H-pyrrolo[2,3-c]pyridine-2-carboxylic acid_(15.6 mg, 0.04 mmol) and 2,2-difluoro-1-methylcyclopropan-1-amine hydrochloride (20.5 mg, 0.14 mmol) and DIPEA (0.019 mL, 0.14 mmol) was reacted to give the title compound (3 mg, 14%).

HPLC t_{R} (Agilent, acidic, 3.5 min): 1.49 min, m/z=527.2 [M+H]⁺.

¹H NMR (400 MHz, DMSO-d₆) δ 12.35 (s, 1H), 8.79 (s, 1H), 7.57 (d, J=7.1 Hz, 1H), 7.43 (s, 1H), 6.96 (d, J=2.2 Hz, 1H), 6.70-6.66 (m, 2H), 6.33 (d, J=7.1 Hz, 1H), 3.55 (s, 3H), 3.45 (s, 3H), 2.00 (s, 6H), 1.71-1.62 (m, 2H), 1.48 (s, 3H).

### Example 45: 4-(4-cyclobutoxythiazol-5-yl)-6-methyl-1,6-dihydro-7H-pyrrolo[2,3-c]pyridin-7-one

### Preparation 156: 4-cyclobutoxythiazole

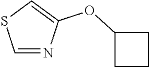

NaH (183 mg, 4.5 mmol) was added to cyclobutanol (1.29 mL, 16.5 mmol) at room temperature and then the resulting solution heated to 60 °C for 1 hour. 4-bromo-thiazole (300 mg, 1.83 mmol) was added and the resulting solution heated to 150 °C for 1 hour. Ethyl acetate (50 ml) was added and the organics washed with 2×50 ml water then 1×50 ml saturated brine solution. The organics were then separated and dried (MgSO₄) before concentration to dryness. The crude was then purified by flash column chromatography eluting with ethyl acetate/heptane gradient (0-100%). The desired fractions were combined and dried to afford was reacted to give the title compound (124 mg, 44%).

¹H NMR (500 MHz, DMSO-d₆) δ 8.52 (s, 1H), 6.04 (s, 1H), 4.80-4.73 (m, 1H), 2.47-2.16 (m, 4H), 1.90-1.81 (m, 1H), 1.70-1.61 (m, 1H).

### Preparation 157:5-bromo-4-cyclobutoxythiazole

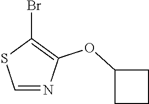 [SMILES: Brc1scnc1OC1CCC1]

Following the procedure in preparation 23, 4-cyclobutoxythiazole (485 mg, 3.1 mmol) was reacted to give the title compound (453 mg, 62%).

¹H NMR (400 MHz, DMSO-d₆) δ 8.42 (s, 1H), 5.03-4.90 (m, 1H), 2.37-2.27 (m, 2H) 2.15-2.05 (m, 2H) 1.79-1.67 (m, 1H), 1.58-1.45 (m, 1H).

### Preparation 158: 4-(4-cyclobutoxythiazol-5-yl)-6-methyl-1,6-dihydro-7H-pyrrolo[2,3-c]pyridin-7-one

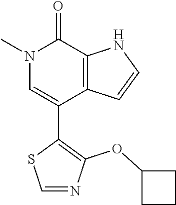 [SMILES: Cn1cc(-c2scnc2OC2CCC2)c2cc[nH]c2c1=O]

Following the procedure in preparation 10, 5-bromo-4-cyclobutoxythiazole (66 mg, 0.28 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (110 mg, 0.026 mmol) was reacted to give the title compound (7 mg, 8%).

HPLC t_{R} (Agilent, acidic, 3.5 min): 1.51 min, m/z=302.1 [M+H]⁺.

¹H NMR (400 MHz, DMSO-d₆) δ 12.17 (bs, 1H), 8.85 (s, 1H), 7.56 (s, 1H), 7.36 (t, J=2.8 Hz, 1H), 6.44 (t, J=2.4 Hz, 1H), 5.12-5.05 (m, 1H), 3.58 (s, 3H), 2.40-2.32 (m, 2H), 2.15-2.05 (m, 2H), 1.81-1.72 (m, 1H), 1.66-1.56 (m, 1H).

### Example 46: 6-methyl-4-(4-propoxythiazol-5-yl)-1,6-dihydro-7H-pyrrolo[2,3-c]pyridin-7-one Preparation 159: 4-propoxythiazole

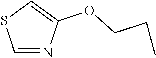

Following the procedure in preparation 156, 1-propanol (3.6 mL, 54.9 mmol) was reacted to give the title compound (150 mg, 28%).

¹H NMR (500 MHz, CDCl₃) δ 8.56 (d, J=2.1 Hz, 1H), 6.14 (d, J=2.3 Hz, 1H), 4.13-4.09 (m, 2H), 1.89-1.82 (m, 2H), 1.07 (t, J=7.5 Hz, 3H).

### Preparation 160:5-bromo-4-propoxythiazole

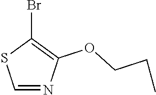

Following the procedure in preparation 23, 4-propoxythiazole_(610 mg, 4.3 mmol) was reacted to give the title compound (592 mg, 62%).

¹H NMR (500 MHz, CDCl₃) δ 8.53 (s, 1H), 4.33-4.29 (m, 2H), 1.82-1.74 (m, 2H), 1.04-1.00 (m, 3H).

### Preparation 161:6-methyl-4-(4-propoxythiazol-5-yl)-1,6-dihydro-7H-pyrrolo[2,3-c]pyridin-7-one

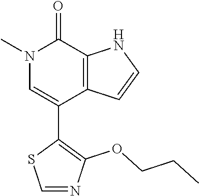 [SMILES: CCCOc1ncsc1-c1cn(C)c(=O)c2[nH]ccc12]

Following the procedure in preparation 10, 5-bromo-4-propoxythiazole (57 mg, 0.26 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (100 mg, 0.023 mmol) was reacted to give the title compound (17 mg, 23%).

HPLC t_{R} (Agilent, acidic, 3.5 min): 1.52 min, m/z=290.1 [M+H]⁺.

¹H NMR (400 MHz, DMSO-d₆) δ 12.19 (bs, 1H), 8.87 (s, 1H), 7.56 (s, 1H), 7.35 (t, J=2.8 Hz, 1H), 6.44 (t, J=2.4 Hz, 1H), 4.31 (t, J=6.5 Hz, 2H), 3.29 (s, 3H), 1.76-1.69 (m, 2H), 0.96 (t, J=7.4 Hz, 3H).

### Example 47: 4-(3-(4-fluoro-2,6-dimethylphenoxy)-1-methyl-2-oxo-1,2-dihydropyridin-4-yl)-6-methyl-1,6-dihydro-7H-pyrrolo[2,3-c]pyridin-7-one

### Preparation 162: 2-chloro-5-(4-fluoro-2,6-dimethylphenoxy)-4-nitropyridine 1-oxide

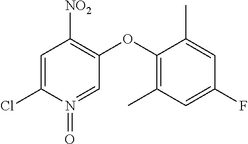 [SMILES: C/C(Oc1c(C)cc(F)cc1C)=C(\C=C(\Cl)N=O)[N+](=O)[O-]]

Following the procedure in preparation 35, 4-fluoro-2,6-dimethylphenol (15.0 g, 77.9 mmol) was reacted to give the title compound (16.0 g, 64%).

¹H NMR (400 MHz, CDCl₃) δ 8.25 (s, 1H), 7.69 (s, 1H), 6.88 (d, J=8.4 Hz, 2H), 2.183 (s, 6H).

### Preparation 163: 2,4-dibromo-5-(4-fluoro-2,6-dimethylphenoxy)pyridine 1-oxide

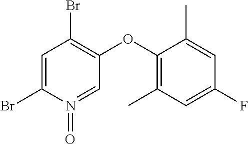 [SMILES: C/C(Oc1c(C)cc(F)cc1C)=C(Br)\C=C(\Br)N=O]

Following the procedure in preparation 26, 2-chloro-5-(4-fluoro-2,6-dimethylphenoxy)-4-nitropyridine 1-oxide (11.0 g, 35.1 mmol) was reacted to give the title compound (11.9 g, 78%).

HPLC t_{R} (Shimadzu, acidic, 1.5 min): 0.93 min, m/z=391.8 [M+H]⁺.

### Preparation 164: 2,4-dibromo-5-(4-fluoro-2,6-dimethylphenoxy)pyridine

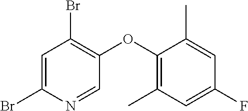 [SMILES: Cc1cc(F)cc(C)c1Oc1cnc(Br)cc1Br]

Following the procedure in preparation 27, 2,4-dibromo-5-(4-fluoro-2,6-dimethylphenoxy)pyridine 1-oxide (17.7 g, 42.5 mmol) was reacted to give the title compound (16.5 g, 65%).

HPLC t_{R} (Shimadzu, acidic, 1.5 min): 1.08 min, m/z=375.8 [M+H]⁺.

### Preparation 165: 4-bromo-5-(4-fluoro-2,6-dimethylphenoxy)pyridin-2 (1H)-one

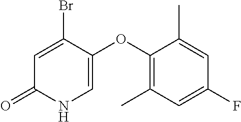 [SMILES: Cc1cc(F)cc(C)c1Oc1c[nH]c(=O)cc1Br]

Following the procedure in preparation 38, 2,4-dibromo-5-(4-fluoro-2,6-dimethylphenoxy)pyridine (7.5 g, 20.0 mmol) was reacted to give the title compound (6.2 g, 99%).

HPLC t_{R} (Shimadzu, acidic, 1.5 min): 0.90 min, m/z=312.0 [M+H]⁺.

### Preparation 166: 4-bromo-5-(4-fluoro-2,6-dimethylphenoxy)-1-methylpyridin-2 (1H)-one

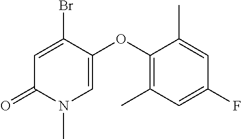 [SMILES: Cc1cc(F)cc(C)c1Oc1cn(C)c(=O)cc1Br]

Following the procedure in preparation 39, 4-bromo-5-(4-fluoro-2,6-dimethylphenoxy)pyridin-2 (1H)-one (6.24 g, 20.0 mmol) was reacted to give the title compound (1.42 g, 22%).

¹H NMR (500 MHz, DMSO-d₆) δ 7.04 (d, J=8.8 Hz, 2H), 6.92 (s, 1H), 6.82 (s, 1H), 3.27 (s, 3H), 2.12 (s, 6H)

### Preparation 167: 4-(3-(4-fluoro-2,6-dimethylphenoxy)-1-methyl-2-oxo-1,2-dihydropyridin-4-yl)-6-methyl-1,6-dihydro-7H-pyrrolo[2,3-c]pyridin-7-one

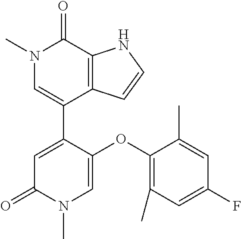 [SMILES: Cc1cc(F)cc(C)c1Oc1cn(C)c(=O)cc1-c1cn(C)c(=O)c2[nH]ccc12]

Following the procedure in preparation 10, 4-bromo-3-(4-fluoro-2,6-dimethylphenoxy)pyridin-2 (1H)-one (152 mg, 0.47 mmol) and 6-methyl-4-(4,4,5,5-tetramethyl-1,3,2-dioxaborolan-2-yl)-1-tosyl-1,6-dihydro-7H-pyrrolo[2,3-c]pyridin-7-one (200 mg, 0.47 mmol) was reacted to give the title compound (77 mg, 42%).

HPLC t_{R} (Agilent, acidic, 3.5 min): 1.36 min, m/z=394.1 [M+H]⁺.

¹H NMR (400 MHz, DMSO-d₆) δ 12.17 (bs, 1H), 7.46 (s, 1H), 7.36-7.33 (m, 1H), 7.00-6.96 (m, 2H), 6.72 (s, 1H), 6.51-6.50 (m, 1H), 6.34 (t, J=2.3 Hz, 1H), 3.58 (s, 3H), 3.34 (s, 3H), 2.09 (s, 6H).

### Primary Activity

The dissociation constant (K_{d}) of Examples 1 to 47 of the compounds described herein, from BRD4 BD1 and BD2 were determined. BRD4 is a representative example of the BET family, as to date highly isoform selective compounds do not exist. Dissociation constants were determined as described below and are represented in Table 1.

### Bromodomain Assay Procedure

T7 phage strains displaying bromodomains were grown in parallel in 24-well blocks in an E. coli host derived from the BL21 strain. E. coli were grown to log-phase and infected with T7 phage from a frozen stock (multiplicity of infection=0.4) and incubated with shaking at 32 °C until lysis (90-150 min). The lysates were centrifuged (5,000×g) and filtered (0.2 μm) to remove cell debris. Streptavidin-coated magnetic beads were treated with biotinylated small molecule or acetylated peptide ligands for 30 min at RT to generate affinity resins for bromodomain assays. The ligated beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining bromodomains, ligated affinity beads, and test compounds in 1× binding buffer (16% SeaBlock, 0.32×PBS, 0.02% BSA, 0.04% Tween 20, 0.004% Sodium azide, 7.9 mM DTT). Test compounds were prepared as 1000×stocks in 100% DMSO and subsequently diluted 1:25 in MEG. The compounds were then diluted directly into the assays such that the final concentrations of DMSO and MEG were 0.1% and 2.4%, respectively. All reactions were performed in polypropylene 384-well plates in a final volume of 0.02 ml. The assay plates were incubated at RT with shaking for 1 hr and the affinity beads were washed with wash buffer (1×PBS, 0.05% Tween 20). The beads were then re-suspended in elution buffer (1×PBS, 0.05% Tween 20, 2 UM non-biotinylated affinity ligand) and incubated at RT with shaking for 30 min. The bromodomain concentration in the eluates was measured by quantitative polymerase chain reaction (qPCR).

An 11-point 3-fold serial dilution of each test compound was prepared in 100% DMSO at 1000× final test concentration. All compounds were distributed by acoustic transfer (non-contact dispensing) in 100% DMSO. The compounds were then diluted directly into the assays such that the final concentration of DMSO was 0.09%. Most dissociation constants were determined using a compound top concentration=10,000 nM. If the initial dissociation constant determined was <0.169 nM (the lowest concentration tested), the measurement was repeated with a serial dilution starting at a lower top concentration.

TABLE 1
Dissociation constants of exemplified compounds from
BRD4 BD1 and BD2

| | BRD4 BD1 | BRD4 BD2 |
|---|---|---|
| Example | K_{d} | K_{d} |
| 1 | ++++ | ++++ |
| 2 | +++ | |
| 3 | +++ | ++++ |
| 4 | +++ | ++++ |
| 5 | +++ | ++++ |
| 6 | ++++ | |
| 7 | +++ | ++++ |
| 8 | +++ | |
| 9 | +++ | |
| 10 | ++++ | |
| 11 | ++++ | |
| 12 | ++++ | |
| 13 | ++ | |
| 14 | ++ | |
| 15 | ++ | |
| 16 | ++ | |
| 17 | +++ | |
| 18 | ++ | |
| 19 | ++ | |
| 20 | +++ | |
| 21 | ++ | |
| 22 | ++ | |
| 23 | +++ | |
| 24 | +++ | |
| 25 | +++ | |
| 26 | +++ | |
| 27 | ++ | |
| 28 | ++ | |
| 29 | +++ | |
| 30 | +++ | |
| 31 | +++ | |
| 32 | +++ | |
| 33 | ++++ | |
| 34 | +++ | |
| 35 | +++ | |
| 36 | ++ | |
| 37 | ++ | |
| 38 | ++ | |
| 39 | + | ++ |
| 40 | ++ | +++ |
| 41 | + | ++++ |
| 42 | + | ++++ |
| 43 | + | +++ |
| 44 | + | +++ |
| 45 | ++ | +++ |
| 46 | ++ | +++ |
| 47 | +++ | ++++ |

Key
+ Kd > 1 μM
++ Kd > 0.1 μM and ≤ 1 μM
+++ Kd > 0.01 μM and ≤ 0.1 μM
++++ Kd ≤ 0.01 μM

Preferably, BET protein inhibitors exhibit a K_{d} of <0.1 μM for BRD4 BD2 or BD1 and BD2. BET protein inhibitors with a K_{d} of <0.1 μM selective for BRD4 BD2 are promising oral drug candidates, whilst BET protein inhibitors with a K_{d} of <0.1 μM selective for BRD4 BD1 and BD2 are promising topical drug candidates.

### BET Selectivity

The selectivity of Examples 1 and 41 of the current invention, against BRD2,3,4 and T BD1 and BD2 were determined as described below and are represented in Table 2.

### Bromodomain Assay Procedure

The same bromodomain assay procedure as that outlined above was used. Example compounds were screened at 30 times their K_{d}, and results for primary screen binding interactions are reported as ‘% Ctrl’, where lower numbers indicate stronger hits in the matrix.

$$\% Ctrl = \frac{test compound signal - positive control signal}{negative control signal - positive control signal} \times 100$$

$$Test compound = A compound of formula ( I ) , such as example 1$$

$$Negative control = DMSO ( 100 \% Ctrl )$$

$$Positive control = control compound ( 0 \% Ctrl )$$

TABLE 2
Single point concentration binding interactions of
exemplified compounds

| | BRD2(1) | BRD3(1) | BRD4(1) | BRDT(1) |
|---|---|---|---|---|
| Example | % Ctrl | % Ctrl | % Ctrl | % Ctrl |
| 1 | 0 | 0 | 0 | 0 |
| 41 | 100 | 85 | 90 | 100 |
| | BRD2(2) | BRD3(2) | BRD4(2) | BRDT(2) |
| Example | % Ctrl | % Ctrl | % Ctrl | % Ctrl |
| 1 | 0.6 | 0.05 | 2.6 | 3.8 |
| 41 | 0.1 | 2.3 | 8.7 | 13 |

Preferably, BET protein inhibitors exhibit a % Ctrl of <10 for BRD 2,3,4 and T BD2 or T BD1 and BD2. BET protein inhibitors with % Ctrl of <10 for BRD 2,3,4 and T BD2 are promising oral drug candidates, whilst BET protein inhibitors with % Ctrl of <10 for BRD 2,3,4 and T BD1 and BD2 are promising topical drug candidates. The data of Table 2 show that Example 41 is a promising oral drug candidate, whilst Example 1 is a promising topical drug candidate.

### BET Selectivity Dose Response

The dissociation constants (K_{d}) of Example 41 of the current invention, from BRD2,3,4 and T BD1 and BD2 was determined as described below and tabulated in Table 3.

### Bromodomain Assay Procedure

The same bromodomain assay procedure as that outlined above was used.

TABLE 3
Dose response binding interactions of exemplified compounds

| | BRD2(1) | BRD3(1) | BRD4(1) | BRDT(1) |
|---|---|---|---|---|
| Example | Kd (nM) | Kd (nM) | Kd (nM) | Kd (nM) |
| 41 | >3000 | >3000 | >3000 | >3000 |
| | BRD2(2) | BRD3(2) | BRD4(2) | BRDT(2) |
| Example | Kd (nM) | Kd (nM) | Kd (nM) | Kd (nM) |
| 41 | <10 | <10 | <10 | <10 |

BET protein inhibitors with a K_{d} of <10 nM for BRD 2,3,4 and selectively T BD2 are promising oral drug candidates. Example 41 exhibits a K_{d} of <10 nM for BRD4 (2) and a K_{d} of >3000 nM for BRD4 (1). Thus, Example 41 is a promising oral drug candidate.

### Cellular Activity—Broad Panel

The EC50 values of Example compounds 1 and 3 of the invention in the reduction of GM-CSF, IL-1a, IL-6, IL-8, CCL2, TNF-a, TSLP, CCL27, CCL20 and CXCL9 levels in primary keratinocytes stimulated by polyinosinic:polycytidylic acid were determined. EC50s were determined as described below and are represented in Table 4.

### Assay Procedure

1. Seed Primary Human Keratinocytes cells (PHK) at 9000 cells/well in a flat bottom 96 well plate.
2. Before treatment, cells must reach a confluence of 90-100% then medium is replaced with fresh medium that does not contain hydrocortisone.
3. Cells are cultured for 24 hr prior to TLR ligand stimulation (polyinosinic:polycytidylic acid).
4. Cells are treated with 20 μg/mL polyinosinic:polycytidylic acid for 48 hrs in 180 μL of media and treated for different compounds or controls.
5. Supernatant is collected and Chemokine and Cytokines analysis is performed by Magpix-Luminex.

### Immunoassay Procedure

### Day 1

1. Add 200 μL of Assay Buffer per well. Shake 10 min, RT. Decant.
2. Add 25 μL of Standard or Control to the appropriate wells.
3. Add 25 μL of Assay Buffer to background and sample wells.
4. Add 25 UL of cell media to background, standard and control wells.
5. Add 25 μL neat samples to sample wells.
6. Add 25 μL of Beads to each well.
7. Incubate overnight (16-18 hr) at 4 °C.

### Day 2

8. Remove well contents and wash 2× with 200 μL Wash buffer.
9. Add 25 μL of Detection Antibodies per well.
10. Incubate 1 hr at RT (20-25 °C).
11. Add 25 μL of Streptavidin-Phycoerytrin per well (do not aspirate).
12. Incubate for 30 min at RT.
13. Remove well contents and wash 2× with 200 UL Wash buffer.
14. Add 150 μL of Wash Buffer per well. Resuspend the beads on a plate shaker for 5 min.
15. Read on Luminex 100 μl (50 beads per bead set).

TABLE 4
EC50 values of exemplified compounds of the invention. The measured drug respons
is the reduction of GM-CSF, IL-1a, IL-6, IL-8, CCL2, TNF-a, TSLP, CCL27, CCL20 and
CXCL9 levels in primary keratinocytes stimulated by polyinosinic:polycytidylic acid.

| Example | GM-CSF | IL-6 | IL-8 | TSLP | IL-1a |
|---|---|---|---|---|---|
| 1 | ++++ | +++ | ++++ | +++ | ++++ |
| 3 | ++++ | +++ | ++++ | ++++ | ++++ |
| Example | TNF-a | CCL2 | CCL20 | CCL27 | CXCL9 |
| 1 | ++++ | +++ | +++ | +++ | ++++ |
| 3 | ++++ | ++++ | ++++ | +++ | ++++ |

Key
+ EC50 > 1 μM
++ EC50 > 0.1 μM and ≤ 1 μM
+++ EC50 > 0.01 μM and ≤ 0.1 μM
++++ EC50 ≤ 0.01 μM

Preferably, BET protein inhibitors exhibit cellular EC50 values of <0.1 μM for one or more of the disease relevant markers in stimulated human primary keratinocytes. Examples 1 and 3 exhibit cellular EC50 values of <0.1 μM in stimulated human primary keratinocytes.

### Cellular Activity—IL-4

The EC50 values of specific Example compounds of the invention in the reduction of IL-4 levels produced by CD4+ T-cells activated with CD2, CD3 and CD28 antibodies were determined as described below and tabulated in Table 5.

### Assay Procedure

1. CD4⁺ T-cells are isolated from cryopreserved human peripheral blood mononuclear cells (PBMCs) using EasySep™ Kit (Cat. No. 17952, Stemcell Technologies).
2, CD2, CD3 and CD28 antibodies coated beads from T cell Activation/Expansion Kit (Cat. No. 130-091-441, Miltenyi Biotec) are added to the CD4⁺ T-cells at a bead-to-cell ratio of 1:2.
3. CD4⁺ T-cells along with the beads are seeded at 2×10⁵ cells/well in a round bottom 96-well plate and treated with different compounds and controls in a total volume of 200 μl.
4. The cells are cultured for 48 hrs at 37 °C, 5% CO₂.
5. Supernatant is collected and IL-4 is analysed by ELISA.

TABLE 5
EC50 values of exemplified compounds of the invention.
The measured drug response is the reduction of IL-4 levels
in CD4+T-cells stimulated by CD2, CD3 and CD28 antibodies

| Example | IL-4 |
|---|---|
| 1 | ++++ |
| 3 | +++ |
| 4 | +++ |
| 5 | ++++ |
| 6 | +++ |
| 7 | +++ |
| 8 | ++ |
| 10 | +++ |
| 18 | + |
| 20 | +++ |
| 24 | ++ |
| 39 | + |
| 40 | ++ |
| 41 | +++ |
| 42 | ++ |
| 43 | ++ |
| 44 | ++ |
| 46 | +++ |
| 47 | ++ |

Key
+ EC50 > 1 μM
++ EC50 > 0.1 μM and ≤ 1 μM
+++ EC50 > 0.01 μM and ≤ 0.1 μM
++++ EC50 ≤ 0.01 μM

Preferably, BET protein inhibitors exhibit cellular EC50 values of <0.1 UM for the reduction of IL-4 levels Examples 1, 3, 4, 5, 6, 7, 10, 20, 41, and 46 exhibit cellular EC50 values of <0.1 μM in CD4+ T-cells stimulated by CD2, CD3 and CD28 antibodies coated beads from T cell Activation/Expansion Kit.

### Human Tissue Data—Th2 and Th17 Stimulation of Human Skin Explants

The % reduction of Example compounds at 2.5 μM listed below of the invention in IL-4 or IL-17A mRNA in healthy human skin stimulated by a Th2 or Th17 biasing cocktail were determined as described below and tabulated in Table 6.

### Assay Procedure

1. Freshly excised healthy human skin tissue from abdominoplasties is defatted, cleaned and sectioned into 7 mm biopsies.
2. The biopsies are placed in Transwell® inserts with the epidermis apical and exposed to air and the dermis submerged in media in the basal chamber.
3. The biopsies are pre-treated overnight at 37 °C, 5% CO₂ with different compounds and controls added to the media in the basal chamber.
4. The next day, contents of the basal chamber is replaced with fresh media containing the test compound and a stimulation cocktail for either Th2 inflammation (proprietary Medpharm cocktail) or Th17 inflammation (mix of antibodies against CD3, CD28, IL-4, IFNγ and recombinant IL-1B, IL-6, IL-21, TGF-β).
5. The biopsies are incubated at 37 °C, 5% CO₂ for a further 24 hrs.
6. After harvesting, the biopsies are cut in half, and one half is homogenized and used for RNA extraction by standard methods. IL-4 or IL-17A is assessed by RT-qPCR.

TABLE 6
% reduction in IL-4 and IL-17A mRNA of compounds
1 and 41 of the invention at 2.5 μM. The measured
drug response is the reduction of IL-4 or IL-17A mRNA
levels in healthy human skin stimulated by a Th2 or Th17
biasing cocktail.

| Example | IL-17 | IL-4 |
|---|---|---|
| 1 | +++ | +++ |
| 41 | +++ | ++ |

Key
+ > 25% reduction
++ > 50% reduction
+++ > 75% reduction

Preferably, BET protein inhibitors exhibit >50% reduction of IL-4 or IL-17 levels, Examples 1 and 41 exhibit >50% reduction in healthy human skin stimulated by a Th2 or Th17 biasing cocktail.

### Intrinsic Clearance in Human Liver Hepatocytes

BET protein inhibitors with a rapid rate of clearance in human liver hepatocytes are promising topical drug candidates. Some of the exemplary compounds of the current invention have rapid clearance in human liver hepatocytes, the rate of which is expressed as a % of liver blood flow. The experimental methods and results (Table 7) are provided hereinafter.

### Assay Procedure

Vials of human cryopreserved hepatocytes, supplied by Life Technologies, were thawed according to manufacturer's instructions and cells re-suspended in Williams Medium E (WME) containing cell maintenance supplement pack (CM4000, Life Technologies). Hepatocytes were incubated in suspension (0.5 million cells/mL) in 48 well non-collagen coated cell culture plates for 10 min at 37 °C, 5% CO₂. Upon addition of an equal volume of supplemented WME containing 1 μM test compound, an aliquot of incubation solution was removed to acetonitrile containing internal standard (final concentration 0.5 μM test compound and a cell density of 0.25 million cells/mL). Similarly, aliquots were removed at 3, 6, 9, 15, 30, 45, 60, 90 and 120 min. 100 UL of 80:20 water:acetonitrile was added to all samples and the analysis plate was centrifuged for 10 min at RT prior to injection and analysis of samples by UPLC-MS/MS. The response (area ratio of test compound to internal standard) was plotted against time using an exponential decay model from which rate of disappearance was calculated.

TABLE 7
Intrinsic clearance (%) of exemplary compounds 1 to 3 in
human liver hepatocytes.

| | % Liver |
|---|---|
| Example | Blood Flow |
| 1 | 83 |
| 2 | 88 |
| 3 | 86 |
| 4 | 80 |
| 5 | <34 |
| 6 | 94 |
| 7 | <34 |
| 8 | 95 |
| 9 | 95 |
| 10 | 93 |
| 11 | 97 |
| 12 | 82 |
| 13 | 34 |
| 18 | 69 |
| 22 | <34 |
| 23 | 38 |
| 24 | 45 |
| 25 | 38 |
| 26 | <34 |
| 27 | <34 |
| 28 | <34 |
| 29 | <34 |
| 30 | <34 |
| 31 | <34 |
| 32 | <34 |
| 33 | <34 |
| 34 | <34 |
| 40 | <34 |
| 41 | <34 |
| 42 | <34 |
| 43 | 38 |
| 44 | 34 |
| 45 | 86 |
| 46 | 89 |

Preferably, BET protein inhibitors for use as topical drugs exhibit intrinsic clearance rates >75% in human liver hepatocytes. Exemplary compounds 1 to 4, 6, 8 to 12, 45 and 46 exhibit intrinsic clearance rates of >75%.

### Solubility in Topical Formulations

Examples 1 to 3 and 6 of the current invention have been shown to have desirable solubility in a range of simple topical formulations. The solubility is expressed in mg/ml. The experimental methods and results are provided hereinafter.

### Assay Procedure

The solubilities of solid exemplary compounds were determined in a selection of solvents and solvent combinations (Transcutol, 50:50 Transcutol:water, Labrasol, propylene glycol and 1:5:4 ethanol:propylene glycol:water), after equilibration. An appropriate volume of each combination was added to a manual weighing of solid compound to provide a 20 mg/ml concentration. The resulting suspension was shaken at 1000 rpm for 5 hr at 32 °C before centrifugation at 13,000×g for 10 min to pellet any precipitate. The supernatant solution was removed and inserted into a HPLC vial and quantified by HPLC-UV against a calibration of a known concentration of the compound in DMSO.

TABLE 8
The solubilities of exemplary compounds of the invention
in various solvents and solvent combinations. TC is Transcutol;
LB is Labrasol; PG is Propylene glycol; EtOH is Ethanol.

| Example | TC | TC:water 1:1 | LB |
|---|---|---|---|
| 1 | +++ | ++ | ++ |
| 2 | +++ | ++ | ++ |
| 3 | +++ | +++ | +++ |
| 6 | +++ | ++ | +++ |

| | | EtOH:PG:water |
|---|---|---|
| Example | PG | 1:5:4 |
| 1 | ++ | ++ |
| 2 | ++ | − |
| 3 | +++ | ++ |
| 6 | +++ | ++ |

Key
+ > 0.1 mg/mL and ≤ 1 mg/mL
++ > 1 mg/mL and ≤ 10mg/mL
+++ > 10mg/mL

Preferably, BET protein inhibitors for use in topical formulations exhibit solubilities of >1 mg/ml of formulation. Exemplary compounds 1 to 3 and 6 exhibit solubilities of >1 mg/mL and in some instances >10 mg/ml of the formulations described.

### Stability in Human Skin S9 Fraction

Exemplary compounds 1 to 3 and 6 have desirable stabilities in human skin S9 fractions. Such fractions model human skin and stability is expressed as the time it takes for the concentration of the compound to decrease by a half (half-life). The experimental methods and some results (Table 9) are provided hereinafter.

### Assay Procedure

An incubation mixture was prepared containing 50 mM potassium phosphate buffer, pH 7.4), 0.3 mg/mL human skin S9 (Sekisui Xenotech), NADPH (final conc 0.8 mg/mL), UDPGA (final conc 0.16 mg/mL) and warmed to 37 °C for 5 min. The reaction was initiated upon addition of test compound (final concentration 0.5 μM). Immediately, at time zero, then at 3, 6, 15, 30, 60, 120 and 180 min, an aliquot (50 μL) of the incubation mixture was removed and mixed with acetonitrile (100 μL) to terminate the reaction. Internal standard was added to all samples, the samples centrifuged to sediment precipitated protein and the plates then sealed prior to UPLC-MS/MS analysis using a Quattro Premier XE (Waters corporation, USA).

Grafit (Erithacus Ltd) was used to calculate the exponential decay and consequently the rate constant (k) from the ratio of peak area of test compound to internal standard at each timepoint. The half life (Ty) of each test compound was determined using the following equation:

T_{1/2}=0.693/k

TABLE 7
T_{1/2} of exemplary compounds in human skin S9 fractions.

| Example | T_{1/2} (min) |
|---|---|
| 1 | >120 |
| 2 | >120 |
| 3 | >120 |
| 6 | >120 |

Preferably, BET protein inhibitors for use in topical formulations exhibit >120 minute T_{1/2} value in human skin. Exemplary compounds 1 to 3 and 6 exhibit T_{1/2} values in human skin S9 fractions of >120 min.

### Hydrolytic Stability at a Range of pHs

Exemplary compounds 1 to 3 of the invention are stable under conditions designed to promote hydrolytic degradation. Stability is expressed as a % decrease after 6 days. The experimental methods and results (Table 8) are provided hereinafter.

### Assay Procedure

To test the hydrolytic stability a 1 mg/mL solution of test materials was made in DMSO (0.1% solution). To 300 μL of each solution in a HPLC vials was added 1200 μL of one of the following:
pH 4.0 buffer—left at 60 °C for 5 days. Samples taken t=0 hr and 6 days
pH 5.5 buffer—left at 60 °C for 5 days. Samples taken t=0 hr and 6 days
pH 7.4 buffer—left at 60 °C for 5 days. Samples taken t=0 hr and 6 days

A 100 μL aliquot was taken at each time point and added to 900 μL of DMSO. This sample was used to determine % decomposition.

% decomposition was measured with a Bruker MicrOTOF II focus ESI Mass Spectrometer connected in parallel to Dionex Ultimate 3000 RSLC system with diode array detector.

TABLE 8
The hydrolytic stability of exemplary compounds of the invention
under conditions designed to promote hydrolytic degradation,
measured at specific pH values and given as % decomposition.

| | pH 4.0 | pH 5.5 | pH 7.4 |
|---|---|---|---|
| | % | % | % |
| Example | decomposition | decomposition | decomposition |
| 1 | 0 | 0 | 0 |
| 2 | 15 | 30 | 0 |
| 3 | 0 | 0 | 0 |

Preferably, BET protein inhibitors exhibit <5% decomposition in conditions designed to promote hydrolytic cleavage. Exemplary compounds 1 to 3 exhibit decompositions of <5% when tested at a pH value of 7.4. Compounds 1 and 3 exhibit decompositions of <5% at all pH values tested.

### Skin Penetration (Franz Cell)

Examples 1 of the invention has desirable skin penetration properties in human skin. Epidermal skin concentrations were determined as described below; the experimental methods and some results (Table 9) are provided hereinafter.

### Assay Procedure

A dosing solution was prepared for each test compound at a saturated concentration in an appropriate formulation mixture. The positive control, caffeine (final concentration 10 mg/mL), was prepared in 50:50 transcutol/water. Warm, degassed phosphate buffered saline (PBS) was applied to the receiving chambers of each jacketed Franz cell (1 cm containing a magnetic stirring bar). Pig/Human skin was removed from −80 °C storage and cut to size (~2 cm²) using a scalpel. The skin was then allowed to thaw at RT before being placed into warmed PBS for 10 min. Each skin piece was then dried before being placed onto the orifice of the Franz cell, removing any bubbles that occurred. The donor chamber was placed onto the skin and clamped in place. 10 μL of dosing solution was then placed onto the skin and parafilm was placed onto the donor chamber to provide occlusion. Using a 1 ml syringe, 200 μL of receiver solution was removed via the sampling arm to a 96 deepwell plate, this was the first time point (To). 200 μL of fresh warmed buffer was added to replace the volume removed. A further 200 μL was removed as described at defined time points over a 24 hr period. 100 μL of each sample was then removed to 100 μL of acetonitrile containing Internal Standard (IS, Donepezil, 4 ng/ml).

After completion, the skin surface was swabbed with a cotton bud to remove any remaining compound. The cotton bud tips were then submerged in DMSO for compound extraction. The skin was removed from the Franz cell and 30 tape strips applied to remove the stratum corneum, which were placed into a vial containing a known volume of DMSO. The skin was then placed surface down onto a heater block at 70 °C for 1 minute after which the epidermis was gradually teased from the dermis using a scalpel. The remaining dermis was cut from the compressed tissue such that only the exposed tissue was left, both pieces of tissue were weighed prior to being placed into individual glass vials and a known volume of DMSO was added.

All skin extraction and wash samples were placed on a shaker for 24 hrs at RT after which, samples were removed to Eppendorfs (where applicable) and centrifuged. Supernatant was removed and diluted appropriately (eg. 1 in 10, 100, 500 and 1000).

A calibration line was prepared in PBS (5000 ng/ml-0.2 ng/ml). 100 μL of each was added to 100 μL of acetonitrile containing IS. All samples were quantified using UPLC-MS/MS (Waters Xevo TQ-S).

The concentration of compound present at each time point was corrected for the addition of fresh buffer. By plotting the concentration of compound vs time, the J flux and T lag could be calculated (values for caffeine should be approximately J Flux: 0.9-1.1 μg/cm/hr, Tlag: 244-257 min, ~20% of dose present in receiving chamber after 24 hrs, mass balance 70-90%).

Skin extraction samples were corrected for dilution factor and volume of extraction solution. The quantity of dose measured in skin layers and time point samples were used to measure the mass balance of the experiment.

The exemplary compound did not penetrate through the skin (so a J flux and T lag value is not given). Rather, the exemplary compound was present in high concentrations in the skin (see Table 9). The mass balance was calculated to be 94%.

TABLE 9
Concentration of exemplary compounds of the
invention in the epidermis of pig/human
skin after 24 hour exposure of the surface of the
skin with the corresponding compound.

| | Epidermal |
|---|---|
| | concentration |
| Example | (μM) |
| 1 | 27 |

### Primary Keratinocyte Cell Viability

The EC50 of exemplary compounds 1 and 3 of the invention in human primary keratinocytes, stimulated by polyinosinic:polycytidylic acid, were determined. EC50s were determined as described below and are represented in Table 10, in which the compound numbers correspond to the numbers in the examples.

### Assay Procedure

1. Seed Primary Human Keratinocytes cells (PHK) at 9000 cells/well in a flat bottom 96 well plate.
2. Before treatment, cells must reach a confluence of 90-100% then medium is replaced with fresh medium that does not contain hydrocortisone.
3. Cells are cultured for 24 hr prior to TLR ligand stimulation (polyinosinic:polycytidylic acid).
4. Cells are treated with 20 μg/ml polyinosinic:polycytidylic acid for 48 hrs in 180 μl of media and treated for different compounds or controls.
6. 20 μL of Cell titre blue reagent together with 100 μL of fresh media is added directly to each well and incubated at 37 °C (cellular incubator) until blue colour turns slightly pink (usually 1 hr).
7. Fluorescence is measured using Citation 3 device. Excitation: 560 nm. Emission: 590 nm.

TABLE 10
The EC50 values of exemplary compounds of the
invention in human primary keratinocytes.

| | Viability |
|---|---|
| Example | (EC50 μM) |
| 1 | >10 |
| 3 | >10 |

Preferably, BET protein inhibitors exhibit a cell viability, EC50 value, of >1 μM. Exemplary compounds 1 and 3 exhibit a cell viability, EC50 value, of >1 μM in human primary keratinocytes.

1. A compound of Formula (II): 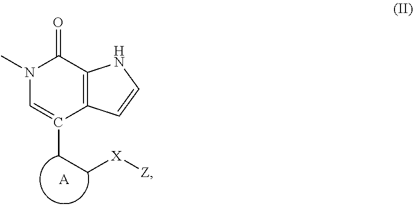
or a stereoisomer or pharmaceutically acceptable salt thereof, wherein:
Ring A is a 5- or 6-membered aromatic or heteroaromatic ring, optionally substituted at one or more carbon and/or heteroatoms with a first substituent;
wherein each first substituent is independently selected from the group consisting of hydroxy, oxo, C₁-C₆alkyl, C₃-C₆cycloalkyl, C₁-C₆alkylol, halo, SO₂C₁-C₄alkyl, NHSO₂C₁-C₄alkyl, SO₂C₃-C₆cycloalkyl, NHSO₂C₃-C₆cycloalkyl, SO₂C₁-C₄alkylol, NHSO₂C₁-C₄alkylol, C₁-C₅alkyloxy, C₁-C₅alkylamino, SO₂NH₂, CONH₂, CONHC₁-C₄alkyl, NHCOC₁-C₄alkyl, NHSO₂N(C₁-C₄alkyl)₂, C₁-C₆fluoroalkyl, SO₂C₁-C₄fluoroalkyl, NHSO₂C₁-C₄fluoroalkyl, C₁-C₅fluoroalkyloxy, and C₁-C₅fluoroalkylamino;
X is O, CR₂, NR′ or S, wherein R is individually selected from the group consisting of H, C₁-C₄alkyl and halo, and R′ is selected from the group consisting of C₁-C₄alkyl and H;
Z is a 5- or 6-membered aromatic or heteroaromatic ring, C₁-C₆alkyl, C₃-C₆cycloalkyl, CR^{A}R^{B}R^{C}, C₂-C₅oxacycloalkyl, C₂-C₅azacycloalkyl or morpholinyl, optionally substituted at one or more carbon and/or heteroatoms with a second substituent;
wherein R^{A} is a C₃-C₅cycloalkyl, R^{B} is a C₃-C₅cycloalkyl, methyl or ethyl, and R^{C} is OH; and
each second substituent is independently selected from the group consisting of hydroxy, C₁-C₆alkyl, C₃-C₆cycloalkyl, halo, C₁-C₅alkyloxy, C₁-C₅alkylamino, oxo, cyano, C₁-C₆fluoroalkyl, C₁-C₅fluoroalkyloxy, and C₁-C₅fluoroalkylamino;
with the proviso that when A is 6-membered, it is substituted at least once with a hydroxy or oxo group.

2. (canceled)

3. The compound of claim 1, wherein Ring A is selected from the group consisting of benzene, pyridine, thiazole, pyridone, pyrazole, imidazole and 1,2,4-triazole, optionally substituted at one or more carbon and/or heteroatoms with the first substituent.

4. The compound of claim 3, wherein the pyridone is a 2-pyridone.

5. The compound of claim 4, wherein the 2-pyridone carbon at position 3 is bound to X and the 2-pyridone carbon at position 4 is bound to C; or
the 2-pyridone carbon at position 5 is bound to X and the 2-pyridone carbon at position 4 is bound to C.

6. The compound of claim 3, wherein:
the thiazole carbon at position 4 is bound to C and the thiazole carbon at position 5 is bound to X;
the pyrazole carbon at position 5 is bound to C and the pyrazole nitrogen at position 1 is bound to X;
the imidazole carbon at position 2 is bound to C and the nitrogen at position 1 is bound to X; or
the 1,2,4-triazole carbon at position 5 is bound to C and the nitrogen at position 1 is bound to X.

7.-9. (canceled)

10. The compound of claim 1, wherein each first substituent is independently selected from the group consisting of hydroxy, oxo, methyl and halo.

11. The compound of claim 1, wherein R is individually selected from the group consisting of H, methyl and fluoro, and R′ is methyl.

12. The compound of claim 1, wherein X is O.

13. The compound of claim 1, wherein Z is a 5- or 6-membered aromatic or heteroaromatic ring, C₁-C₆alkyl, or C₃-C₆cycloalkyl, optionally substituted with a second substituent.

14. (canceled)

15. The compound of claim 1, wherein Z is a phenyl or pyridyl ring, optionally substituted with a second substituent.

16. The compound of claim 1, wherein each second substituent is independently selected from the group consisting of hydroxy, C₁-C₄alkyl and halo.

17. The compound of claim 1, wherein each second substituent is independently selected from hydroxy, methyl and fluoro.

18. The compound of claim 1, wherein C-A-X of Formula (II) is any one of formulae (Ia), (Ib), (Ic), (Id) or (Id′): 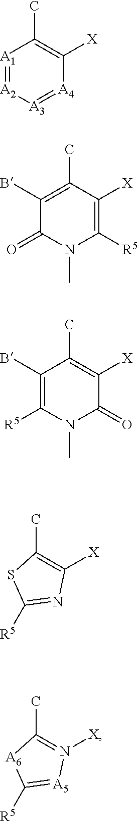
wherein A₁ is CR¹ or N, A₂ is CR² or N, A₃ is CR³ or N, A₄ is CR⁴, As is CR⁵ or N and A₆ is CR⁵ or N;
R¹ is H or hydroxy;
R² is H, hydroxy, C₁-C₆alkyl, C₃-C₆cycloalkyl, halo, SO₂C₁-C₄alkyl, NHSO₂C₁-C₄alkyl, SO₂C₃-C₆cycloalkyl, NHSO₂C₃-C₆cycloalkyl, C₁-C₅alkyloxy or C₁-C₅alkylamino;
R³ and R⁴ are independently selected from the group consisting of H, hydroxy, C₁-C₆alkyl, C₃-C₆cycloalkyl, halo, C₁-C₅alkyloxy or C₁-C₅alkylamino;
with the proviso that at least one of R¹, R², R³ or R⁴ is hydroxy;
B′ is H or hydroxy; and
R⁵ is either H or the first substituent, as defined in claim 1.

19. The compound of claim 18, wherein R² is H, C₁-C₃alkyl, halo, SO₂C₁-C₄alkyl or NHSO₂C₁-C₄alkyl; and R³ and R⁴ are independently selected from the group consisting of H, hydroxy, C₁-C₃alkyl and halo.

20. The compound of claim 18, wherein;
when CAX is represented by formula (Ia), Z is a phenyl ring, substituted at one or more carbon atoms with a second substituent;
when CAX is represented by any one of formulae (Ib), (Ic), (Id) and (Id′), Z is a phenyl or pyridyl ring, optionally substituted at one or more carbon and/or nitrogen atoms with a second substituent selected from the group consisting of hydroxy, methyl, fluoro and chloro; or
when CAX is represented by formula (Ia), Z is an unsubstituted phenyl ring.

21.-22. (canceled)

23. A compound selected from any one of formulae (Ie) to (IIi): 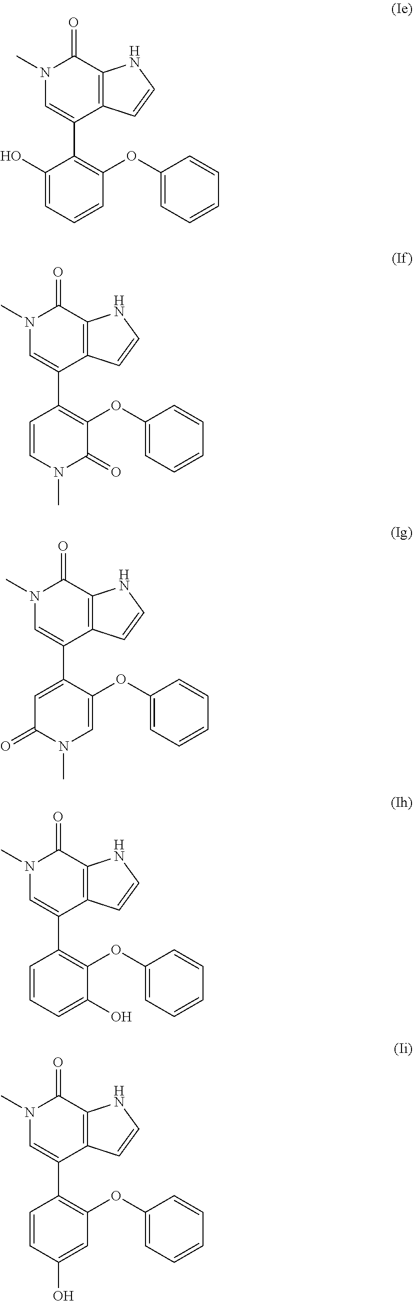 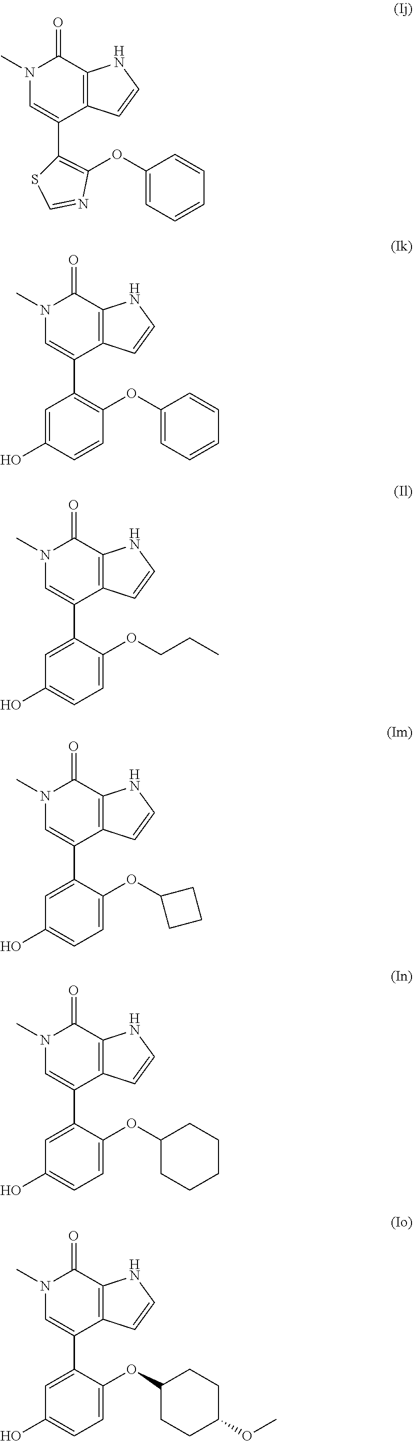 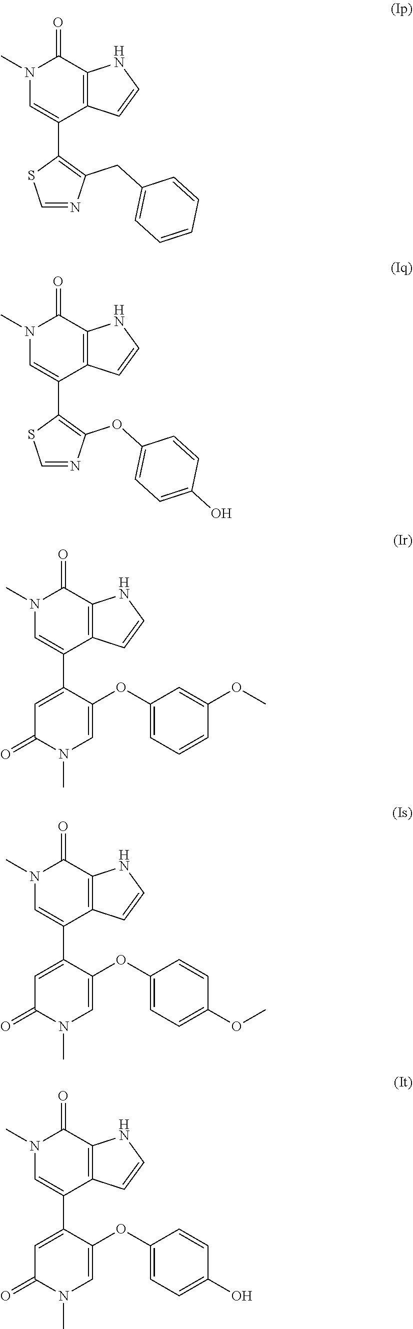 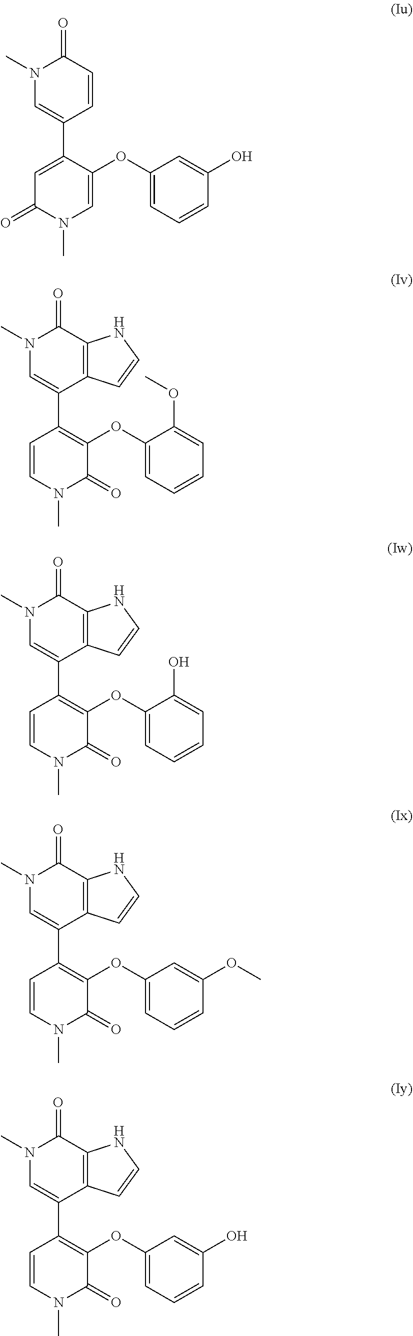 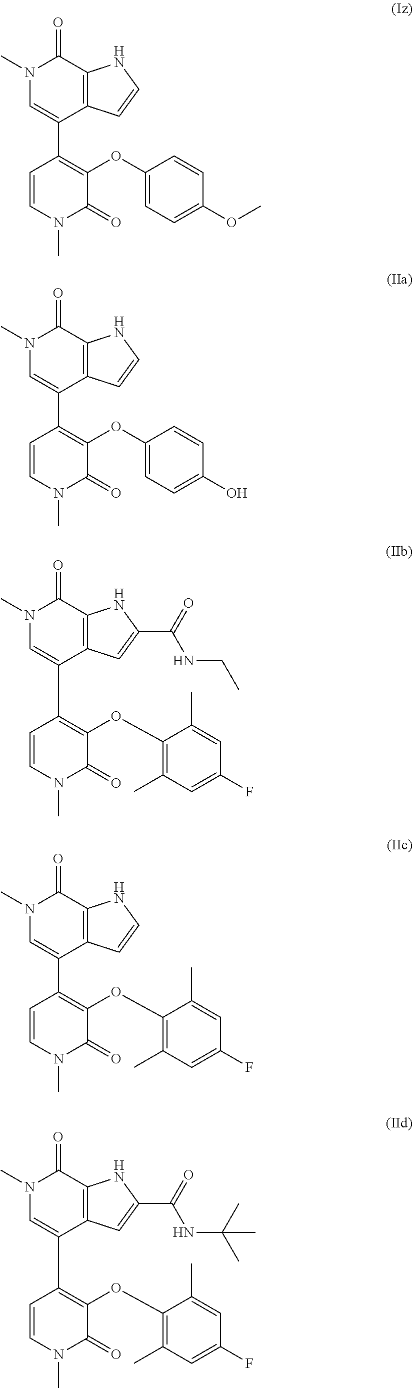 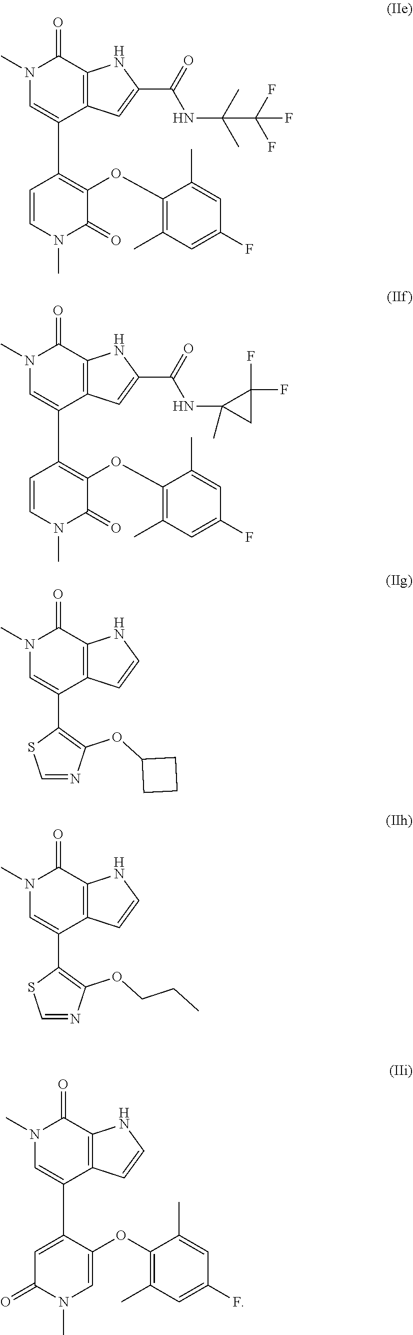
or a pharmaceutically acceptable salt thereof.

24. (canceled)

25. A pharmaceutical composition comprising a compound of claim 23, or a stereoisomer or pharmaceutically acceptable salt thereof, in combination with one or more pharmaceutically acceptable excipients.

26.-29. (canceled)

30. A method for the treatment or prophylaxis of inflammatory skin disorders, respiratory diseases, gastrointestinal diseases, eye diseases, cancers, rheumatic diseases, demyelinating diseases and fibrotic diseases, the method comprising administering to a subject a compound of claim 23, or a stereoisomer or pharmaceutically acceptable salt thereof.

31. The method according to claim 30, wherein the method is for the treatment or prophylaxis of fibrosis of inflammation or cancer of the gut, skin, or lung.

32. A method of inhibiting Bromodomain and Extra-Terminal protein activity in a subject, said method comprising administering to a subject an effective amount of a compound of claim 23, or a stereoisomer or pharmaceutically acceptable salt thereof.
